(19) United States
(12) Patent Application Publication
(10) Pub. No.: US 2013/0209406 A1
(43) Pub. Date: Aug. 15, 2013

(54) RECOMBINANT RNA VIRUSES AND USES THEREOF

(71) Applicant: Benjamin R. tenOever, New York, NY (US)

(72) Inventor: Benjamin R. tenOever, New York, NY (US)

(21) Appl. No.: 13/702,532

(22) PCT Filed: Jun. 6, 2011
(86) PCT No.: PCT/US11/39284

§ 371 Date: Feb. 15, 2013

Related U.S. Application Data
(60) Provisional application No. 61/351,908, filed on Jun. 6, 2010.

(51) Int. Cl. C12N 7/00

(52) U.S. Cl. CPC C12N 7/00
USPC 424/93.2; 536/23.1; 435/235.1

(57) ABSTRACT

Described herein are modified RNA virus gene segments and nucleic acids encoding modified RNA virus gene segments. Also described herein are recombinant RNA viruses comprising modified RNA virus gene segments and the use of such recombinant RNA viruses for the prevention and treatment of disease.

This application claims priority benefit of U.S. provisional application No. 61/351,908, filed Jun. 6, 2010, which is incorporated herein by reference in its entirety.

This invention was made with government support under award number number W911NF-07-R-0001-05 from the Army Research Office. The government has certain rights in this invention.

### 1. INTRODUCTION

Described herein are modified RNA virus gene segments and nucleic acids encoding modified RNA virus gene segments. Also described herein are recombinant RNA viruses comprising modified RNA virus gene segments and the use of such recombinant RNA viruses for the prevention and treatment of disease. Further described herein is the use of RNA viruses for the delivery of RNA molecules that interfere with the expression of disease-related genes.

### 2. BACKGROUND

Viruses capable of producing RNA sequences that adeptly modulate messenger RNA, e.g., miRNAs, would represent a valuable resource in combating diseases and disorders and in amplifying the host response to virus being used in vaccinations. Indeed, the issue of effective and non-toxic delivery of miRNAs is a key challenge and serves as the most significant barrier between RNA interference (RNAi) technology and its therapeutic application (see, e.g., Mittal (2004) Nat Rev Genet 5(5):355-365; and Grimm (2009) Advanced Drug Delivery Reviews 61:672-703). While lentivirus- and lipid-based-delivery models have demonstrated some in vivo success, genomic integration and/or insufficient generation of intracellular miRNAs have limited their applications (see, e.g., Mittal (2004) Nat Rev Genet 5(5):355-365). In contrast, non-integrating viral vectors have been found to induce ultraphysiological and sustained levels of small RNAs resulting in toxicity through saturation of the host small RNA cell machinery (see, e.g., Grimm et al. (2006) Nature 441(7092):537-541). As such, there remains a need for a virus-based RNA delivery system comprising virus with the ability to induce high, transient levels of RNA sequences that can be utilized to treat disease (see, e.g., Zeng et al. (2002) Mol Cell 9(6):1327-1333).

### 3. SUMMARY

This application is based, in part, on the discovery that RNA viruses can be engineered to produce heterologous RNA sequences (e.g., microRNA, small interfering RNA, antisense RNA, small hairpin RNA) involved in post-transcriptional gene silencing (PTGS). In certain aspects, these recombinant RNA viruses do not undergo genomic integration and are able to replicate normally in subjects, and therefore represent superior viruses for delivery of heterologous RNA sequences involved in post-transcriptional gene processing to a subject for, e.g., the prevention or treatment of disease and for enhancing the host immune response to vaccinations.

In one aspect, provided herein is a chimeric viral genomic segment, wherein the chimeric viral genomic segment is derived from an RNA virus and wherein the chimeric viral genomic segment comprises a heterologous RNA, wherein the heterolgous RNA is transcribed in a cell to give rise to an effector RNA that interferes with the expression of a target gene in the cell. In one embodiment, the RNA virus is a segmented, single-stranded, negative sense RNA virus or a segmented double stranded RNA virus. In another embodiment, the effector RNA is an miRNA, a mirtron, an shRNA, an siRNA, a piRNA, an svRNA, or an antisense RNA. In certain embodiments, the virus from which the chimeric viral genomic segment is derived is an orthomyxovirus, a bunyavirus, or an arenavirus.

In a specific embodiment, the chimeric virus gene segment comprises: (a) packaging signals found in the 3′ non-coding region of an orthomyxovirus gene segment; (b) a first nucleotide sequence that forms part of an open reading frame of an orthomyxovirus virus gene; (c) a splice donor site; (d) a heterologous RNA sequence; (e) a splice acceptor site; and (f) a second nucleotide sequence that forms part of the open reading frame of the orthomyxovirus virus gene; and (g) packaging signals found in the 5′ non-coding region of an orthomyxovirus virus gene segment.

In another specific embodiment, the chimeric virus gene segment comprises: (a) packaging signals found in the 3′ non-coding region of an orthomyxovirus virus gene segment; (b) a first nucleotide sequence that forms part of an open reading frame of a first orthomyxovirus virus gene and a second influenza virus gene; (c) a splice donor site; (d) a second nucleotide sequence that forms part of the open reading frame of the first orthomyxovirus virus gene; (e) a heterologous RNA sequence; (e) a splice acceptor site; (f) a third nucleotide sequence that forms part of the open reading frame of the second orthomyxovirus virus gene; and (g) packaging signals found in the 5′ non-coding region of an orthomyxovirus virus gene segment. In a specific embodiment, the first orthomyxovirus virus gene is the influenza virus NS1 gene and the second orthomyxovirus virus gene is the influenza virus NS2 gene. In another specific embodiment, the first orthomyxovirus virus gene is the influenza virus M1 gene and the second orthomyxovirus virus gene is the influenza virus M2 gene.

In another aspect, provided herein is a chimeric viral genome, wherein the chimeric viral genome is derived from an RNA virus and wherein the chimeric viral genome comprises a heterologous RNA, wherein the heterolgous RNA is transcribed in a cell to give rise to an effector RNA that interferes with the expression of a target gene in the cell. In one embodiment, the RNA virus is a non-segmented, single stranded, negative sense RNA virus. In another embodiment, the RNA virus is a non-segmented, single stranded, positive sense RNA virus. In another embodiment, the effector RNA is an miRNA, a mirtron, an shRNA, an siRNA, a piRNA, an svRNA, or an antisense RNA. In certain embodiments, the virus from which the chimeric viral genome is derived is a rhabdovirus, a paramyxovirus, a Filovirus, a hepatitis delta virus, a bornavirus, a picornavirus, a togavirus, a flavivirus, a coronavirus, a reovirus, a rotavirus, an orbivirus, or a Colorado tick fever virus

Also provided herein are recombinant RNA viruses comprising the chimeric viral genomic segments and the chimeric viral genomes provided herein. In specific embodiments, the recombinant RNA viruses are attenuated.

Also provided herein are nucleic acids encoding the chimeric viral genomic segments and the chimeric viral genomes provided herein. In specific embodiments, the nucleic acid is DNA.

Also provided herein are methods of making the recombinant RNA viruses of described herein, wherein said methods comprise introducing the nucleic acids described herein into a cell that expresses all other components for generation of the recombinant RNA virus; and purifying the recombinant RNA virus from the supernatant of the cell.

Also provided herein are substrates, e.g., an egg or a cell, comprising the chimeric viral genomic segments described herein or the chimeric viral genomes described herein; or the recombinant RNA viruses described herein.

Also provided herein are pharmaceutical compositions and immunogenic compositions comprising the recombinant RNA viruses described herein.

Also provided herein are methods of treating and/or preventing a disease in a subject, said methods comprising administering a recombinant RNA virus described herein to the subject, wherein the effector RNA produced by the recombinant RNA virus interferes with expression of a gene that is overexpressed or ectopically expressed in the disease.

Also provided herein are kits comprising one or more of the recombinant RNA viruses described herein, the chimeric viral genomic segments described herein, and/or the chimeric viral genomes described herein.

### 3.1 TERMINOLOGY

As used herein, the term “about” or “approximately” when used in conjunction with a number refers to the number referenced or to any number within 1, 5 or 10% of the referenced number.

As used herein, the terms “disease” and “disorder” are used interchangeably to refer to a condition in a subject. Exemplary diseases/disorders that can be treated in accordance with the methods described herein include cancer, viral infections, bacterial infections, and genetic disorders.

As used herein, the term “effective amount” in the context of administering a therapy to a subject refers to the amount of a therapy which has a prophylactic and/or therapeutic effect(s). In certain embodiments, an “effective amount” in the context of administration of a therapy to a subject or a population of subjects refers to the amount of a therapy which is sufficient to achieve one, two, three, four, or more of the following effects: (i) reduce or ameliorate the severity of a disease in the subject or population of subjects or a symptom associated therewith; (ii) reduce the duration of a disease in the subject or population of subjects or a symptom associated therewith; (iii) prevent the progression of a disease in the subject or population of subjects or a symptom associated therewith; (iv) cause regression of a disease in the subject or population of subjects or a symptom associated therewith; (v) prevent the development or onset of a disease in the subject or population of subjects or a symptom associated therewith; (vi) prevent the recurrence of a disease in the subject or population of subjects or a symptom associated therewith; (vii) prevent or reduce the spread of a disease from the subject or population of subjects to another subject or population of subjects; (viii) reduce organ failure associated with a disease in the subject or population of subjects; (ix) reduce the incidence of hospitalization of the subject or population of subjects; (x) reduce hospitalization length of the subject or population of subjects; (xi) increase the survival of the subject or population of subjects; (xii) eliminate a disease in the subject or population of subjects; (xiii) enhance or improve the prophylactic or therapeutic effect(s) of another therapy in the subject or population of subjects; (xiv) prevent the spread of a virus or bacteria from a cell, tissue, organ of the subject to another cell, tissue, organ of the subject; and/or (xv) reduce the number of symptoms of a disease in the subject or population of subjects.

As used herein, the term “recombinant RNA virus” refers to a virus described herein that comprises heterologous RNA. Recombinant RNA viruses do not include retroviruses.

As used herein, the term “target gene” refers to a gene in a subject or plant to which an effector RNA produced by a recombinant RNA virus is directed. In some embodiments, a target gene is a gene associated with a disease, i.e., the expression of the target gene is implicated in pathogenesis of the disease. In some embodiments, a target gene is a gene of a pathogen, e.g., the target gene is a gene essential to the replication or survival of the pathogen.

As used herein, in some embodiments, the term “wild-type” in the context of a virus, refers to the types of a virus that are prevalent, circulating naturally and producing typical outbreaks of disease. In other embodiments, the term “wild-type” in the context of a virus refers to a parental virus.

As used herein, the term “heterologous RNA” refers to an RNA sequence that has been introduced into the genome of an RNA virus and that is not part of the genome of the wild type RNA virus. Transcription of heterologous RNA, and optionally processing of the resulting transcript, yields an effector RNA.

The term “effector RNA,” as used herein, refers to the RNA molecule that results from transcription, optionally processing, of heterologous RNA and that interferes with the expression of a gene.

As used herein, the term “post-transcriptional gene silencing,” abbreviated as PTGS, refers to the modification of genes following transcription of the DNA sequence that corresponds to the gene.

As used herein, the terms “hybridize,” “hybridizes,” and “hybridization” refer to the annealing of complementary nucleic acid molecules. In certain embodiments, the terms “hybridize,” “hybridizes,” and “hybridization” as used herein refer to the binding of two or more nucleic acid sequences that are at least 60% (e.g., 70%, 75%, 80%, 85%, 90%, 95%, 98%, 99% or 99.5%) complementary to each other. In certain embodiments, the hybridization is under high stringency conditions. In certain embodiments the hybridization is under moderate (i.e., medium) stringency conditions. In certain embodiments the hybridization is under low stringency conditions. In some embodiments, two nucleic acids hybridize to one another if they are not fully complementary, for example, they hybridize under low- to medium-stringency conditions. Those of skill in the art will understand that low, medium and high stringency conditions are contingent upon multiple factors all of which interact and are also dependent upon the specific properties of the nucleic acids involved. In certain embodiments, a nucleic acid hybridizes to its complement only under high stringency conditions. For example, typically, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents. Nucleic acid hybridization techniques and conditions are known in the art and have been described, e.g., in Sambrook et al. Molecular Cloning A Laboratory Manual, 2nd Ed. Cold Spring Lab. Press, December 1989; U.S. Pat. Nos. 4,563,419 and 4,851,330, and in Dunn et al., 1978, Cell 12: 23-26, among many other publications. Various modifications to the hybridization reactions are known in the art.

As used herein, the term “in combination,” in the context of the administration of two or more therapies to a subject, refers to the use of more than one therapy (e.g., more than one prophylactic agent and/or therapeutic agent). The use of the term “in combination” does not restrict the order in which therapies are administered to a subject. For example, a first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, the term “viral infection” means the invasion by, multiplication and/or presence of a virus in a cell or a subject. In one embodiment, a viral infection is an “active” infection, i.e., one in which the virus is replicating in a cell or a subject. Such an infection is characterized by the spread of the virus to other cells, tissues, and/or organs, from the cells, tissues, and/or organs initially infected by the virus. An infection may also be a latent infection, i.e., one in which the virus is not replicating.

As used herein, the term “bacterial infection” means the invasion by, multiplication and/or presence of a bacteria in a cell or a subject.

As used herein, the term “pathogen infection” means the invasion by, multiplication and/or presence of a pathogen in a cell or a subject.

As used herein, the term “influenza virus disease” refers to the pathological state resulting from the presence of an influenza (e.g., influenza A or B virus) virus in a cell or subject or the invasion of a cell or subject by an influenza virus. In specific embodiments, the term refers to a respiratory illness caused by an influenza virus.

As used herein, the term “virus disease” refers to the pathological state resulting from the presence of a virus in a cell or subject or the invasion of a cell or subject by a virus.

As used herein, the numeric term “log” refers to log₁₀.

As used herein, the phrase “multiplicity of infection” or “MOI” is the average number of infectious virus particles per infected cell. The MOI is determined by dividing the number of infectious virus particles added (ml added×PFU/ml) by the number of cells added (ml added×cells/ml).

As used herein, the term “nucleic acid” refers to deoxyribonucleotides, deoxyribonucleic acids, ribonucleotides, and ribonucleic acids, and oligomeric and polymeric forms thereof, and analogs thereof, and includes either single- or double-stranded forms. Nucleic acids include naturally occurring nucleic acids, such as deoxyribonucleic acid (“DNA”) and ribonucleic acid (“RNA”) as well as nucleic acid analogs. Nucleic acid analogs include those which contain non-naturally occurring bases, nucleotides that engage in linkages with other nucleotides other than the naturally occurring phosphodiester bond or which contain bases attached through linkages other than phosphodiester bonds. Thus, nucleic acid analogs include, for example and without limitation, locked-nucleic acids (LNAs), peptide-nucleic acids (PNAs), morpholino nucleic acids, glycolnucleic acid (GNA), threose nucleic acid (TNA), phosphorothioates, phosphorodithioates, phosphorotriesters, phosphoramidates, boranophosphates, methylphosphonates, chiral-methyl phosphonates, 2-O-methyl ribonucleotides, and the like. In certain embodiments, as used herein, the term “nucleic acid” refers to a molecule composed of monomeric nucleotides.

As used herein, the terms “prevent,” “preventing” and “prevention” in the context of the administration of a therapy(ies) to a subject to prevent a disease refers to one or both of the following effects resulting from the administration of a therapy or a combination of therapies: (i) the inhibition of the development or onset of the disease or a symptom thereof; and (ii) the inhibition of the recurrence of the disease or a symptom associated therewith.

As used herein, the terms “purified” and “isolated” when used in the context of a protein or nucleic acid that is obtained from a natural source, e.g., cells, refers to a polypeptide which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a protein or nucleic acid that is isolated includes preparations of a protein or nucleic acid having less than about 30%, 20%, 10%, 5%, 2%, or 1% (by dry weight) of cellular materials and/or contaminating materials. As used herein, the terms “purified” and “isolated” when used in the context of a protein or nucleic acid that is chemically synthesized refers to a protein or nucleic acid which is substantially free of chemical precursors or other chemicals which are involved in the syntheses of the polypeptide.

As used herein, the terms “viral replication” and “virus replication” refer to one or more, or all, of the stages of a viral life cycle which result in the propagation of virus. The steps of a viral life cycle include, but are not limited to, virus attachment to the host cell surface, penetration or entry of the host cell (e.g., through receptor mediated endocytosis or membrane fusion), uncoating (the process whereby the viral capsid is removed and degraded by viral enzymes or host enzymes thus releasing the viral genomic nucleic acid), genome replication, synthesis of viral messenger RNA (mRNA), viral protein synthesis, and assembly of viral ribonucleoprotein complexes for genome replication, assembly of virus particles, post-translational modification of the viral proteins, and release from the host cell by lysis or budding and acquisition of a phospholipid envelope which contains embedded viral glycoproteins. In some embodiments, the terms “viral replication” and “virus replication” refer to the replication of the viral genome. In other embodiments, the terms “viral replication” and “virus replication” refer to the synthesis of viral proteins.

As used herein, the terms “subject” or “patient” are used interchangeably to refer to an animal (e.g., birds, reptiles, and mammals). In a specific embodiment, a subject is a bird (e.g., chicken or duck). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal (e.g., cow, pig, horse, sheep, goat, etc.) or pet (e.g., dog, cat, etc.). In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

As used herein, the term “premature human infant” refers to a human infant born at less than 37 weeks of gestational age.

As used herein, the term “human infant” refers to a newborn to 1 year old human.

As used herein, the term “human toddler” refers to a human that is 1 years to 3 years old.

As used herein, the term “human child” refers to a human that is 1 year to 18 years old.

As used herein, the term “human adult” refers to a human that is 18 years or older.

As used herein, the term “elderly human” refers to a human 65 years or older.

As used herein, the terms “therapies” and “therapy” can refer to any protocol(s), method(s), compound(s), composition(s), formulation(s), and/or agent(s) that can be used in the prevention or treatment of a disease or symptom associated therewith. In certain embodiments, the terms “therapies” and “therapy” refer to biological therapy, supportive therapy, and/or other therapies useful in treatment or prevention of a disease or symptom associated therewith known to one of skill in the art. In some embodiments, a therapy does not result in a cure for a disease.

As used herein, the terms “treat,” “treatment,” and “treating” refer in the context of administration of a therapy(ies) to a subject or a population of subjects to treat a disease to obtain a beneficial or therapeutic effect of a therapy or a combination of therapies. In specific embodiments, such terms refer to one, two, three, four, five or more of the following effects resulting from the administration of a therapy or a combination of therapies: (i) reduction or amelioration of the severity of a disease in the subject or population of subjects or a symptom associated therewith; (ii) reduction of the duration of a disease in the subject or population of subjects or a symptom associated therewith; (iii) prevention of the progression of a disease in the subject or population of subjects or a symptom associated therewith; (iv) regression of a disease in the subject or population of subjects or a symptom associated therewith; (v) prevention of the development or onset of a disease in the subject or population of subjects or a symptom associated therewith; (vi) prevention of the recurrence of a disease in the subject or population of subjects or a symptom associated therewith; (vii) prevention or reduction of the spread of a disease from the subject or population of subjects to another subject or population of subjects; (viii) reduction in organ failure associated with a disease in the subject or population of subjects; (ix) reduction of the incidence of hospitalization of the subject or population of subjects; (x) reduction of the hospitalization length of the subject or population of subjects; (xi) an increase the survival of the subject or population of subjects; (xii) elimination of a disease in the subject or population of subjects; (xiii) enhancement or improvement of the prophylactic or therapeutic effect(s) of another therapy in the subject or population of subjects; (xiv) prevention of the spread of a pathogen from a cell, tissue, organ of the subject to another cell, tissue, organ of the subject; and/or (xv) reduction of the number of symptoms of a disease in the subject or population of subjects.

As used herein, the term “population of subjects” refers to a group of at least 5 subjects to which a therapy(ies) has been administered. In certain embodiments, a population of subjects is at least 10 subjects, at least 25 subjects, at least 50 subjects, at least 100, at least 500, at least 1000, or between 10 to 25 subjects, 25 to 50 subjects, 50 to 100 subjects, 100 to 500 subjects, or 500 to 1000 subjects.

### 4. BRIEF DESCRIPTION OF THE DRAWINGS

FIG. 1: Engineered split NS1/NEP viruses do not impact viral replication. (A) Top: Diagram of original NS vRNA segment as compared to engineered split NS1/NEP construct. Middle: Diagram of NS1 and NEP mRNA from engineered split vRNA and plasmid encoding a spliced RFP construct for delivery of exogenouos miRNA. Bottom: Diagram of scrambled (scbl), pri-miR-124 or pri-miR-124(R) inserts. Asterisks show where inserts were ligated (B) Small northern blot of plasmid- and virus-based (MOI 2) miR-124 expression of scbl, miR-124, and miR-124(R). Levels of miR-93 and U6 were used as loading controls. (C) Western blot analysis of mock or scbl, miR-124 or wild type A/PR/8/34 influenza (wt) virus infections in MDCK cells (MOI 2). Blots depict viral nucleoprotein (NP), non-structural protein 1 (NS1), nuclear export protein (NEP/NS2) and Actin. (D) Multi-cycle growth curve on viruses from (C) performed in MDCK cells. Error bars depict standard deviation of triplicate samples.

FIG. 2: Engineered viral synthesis of miR-124. (A) Small Northern blot of viral miR-124 at hours post infection indicated (MOI 1). Levels of miR-93 and U6 were used as loading controls. (B) qRT-PCR analysis of viral miR-124 levels standardized with small nucleolar RNA-202 (snoRNA-202). (C) qRT-PCR analysis of viral PB2 levels standardized with tubulin. Error bars indicate standard deviation. (D) Northern blot of viral miR-124 levels in wild type and Dcr1^{-/-} fibroblasts. (E) qRT-PCR of samples generated in (D).

FIG. 3: Viral genomic miRNA hairpins are not substrates for Drosha. (A) Diagram of miR-124 producing segment eight. RNA species include vRNA, cRNA, and mRNA. Primers and reference numbers used in subsequent experiments are depicted. Primers used in reverse transcription (RT) are as follows: RT1 represents oligo dT, RT2 is specific to the non-coding region of NS cRNA. (B) RT-PCR products of NEP/NS2 mRNA and NS1 mRNA/3′ NS cRNA. RT1 and RT2 depict primers used in the reverse transcription reaction. RNA was derived from mock infected fibroblasts (−) or cells treated (+) with wild type influenza A/PR/8/34. (C) qPCR from mock treated fibroblasts or cells infected with either scbl or miR-124-producing influenza A viruses. Values depict 5′ NS cRNA levels as compared to tubulin. (D) Same as in (C) using 3′ cRNA-specific primers. (E) Same as in (D) using primers specific to the pri-miR-124 insert. (F) 5′ RACE analysis of viral infection. Indicated gel sections were purified and sequenced, representative results denoted in the table with reference to numbered diagram in (A).

FIG. 4: Viral genomic RNA is not targeted by miRNAs. (A) Diagram of recombinant segment eight encoding an untargeted scrambled insert (scbl) or miR-142 target sites oriented to either the NS vRNA (vRNAt) or the NS1 mRNA (mRNAt). (B) Small Northern blot probed for miR-142 expression in cells transfected with a miR-142 expression vector. (C) Western blot of MDCK cells, and MDCK cells stably expressing miR-142, mock treated or infected with scbl, vRNAt, or mRNAt viruses (MOI 0.1). Immunoblots for NS1, NP, and actin depicted.

FIG. 5: Engineered influenza virus produces functional miR-124. (A) Fibroblasts, transfected with a miR-124 targeted GFP construct, were infected with scrambled (scbl) or miR-124-producing (miR-124) influenza A viruses and compared to untreated cells. FACS analysis was used to determine GFP expression (36 hours post-infection). (B) CAD cells were fixed either following 48 hr serum-starvation or 24 hours post infection (MOI 1) with either scrambled (scbl) or miR-124-producing virus. Cells were stained with β-tubulin prior to imaging by confocal microscopy. Hoechst dye used to visualize nuclei.

FIG. 6: miR-124 is not produced from NS1 UTR. (A) Diagram of plasmid-expressing NS1 with a mir-124 hairpin in the 3′ UTR. (B) Western blot analysis of mock, scbl, and UTR transfected cells. Fibroblasts were harvested 24 hours post transfection. Blots depict NS1 protein and actin. (C) qRT-PCR analysis of miR-124 levels of samples from (B) plus full length NS1/NEP 124, standardized with snoRNA-202.

FIG. 7: (A) Cartoon schematic of recombinant Sindbis virus indicating the subgenomic insertion point for the miR-124 locus (Sindbis-124). (B) Confocal microscopy of CAD cells. Left panel: mock infected CAD cells. Right panel: Sindbis-124 infected CAD cells 36 hours post infection. (C) Northern blot of human 293 fibroblasts infected with Sindbis-124 or a Sindbis virus encoding a scrambled (scbl) RNA locus. Transfection of a miR-124 producing plasmid was used as a positive control.

FIG. 8: Schematic representation of generation of heterologous RNA from recombinant orthomyxoviruses. Abbreviations are as follows: vRNA is the viral genomic RNA; mRNA is the transcribed messenger RNA; U_{N} designates a stretch of uridine residues; A_{N} designates a stretch of adenine residues. A) Recombinant orthomyxovirus genome segment (vRNA (modified)) resulting in heterologous RNA upon transcription and splicing. B) Recombinant orthomyxovirus genome segment (vRNA (modified)) resulting in heterologous RNA upon transcription and ribozyme activity.

FIG. 9: Schematic representation of generation of heterologous RNA from recombinant non-segmented RNA viruses. Abbreviations are as follows: 5′ C designates the 5′ cap; ns designates non-structural proteins; P123 designates polyprotein 123; NCR designates non-coding region; CP, E3, E2, 6K, and E1 are structural proteins; N, P, M, G, and L are viral genes; A_{N} designates a stretch of adenine residues. A) Generation of heterologous RNA from recombinant Togaviridae. B) Generation of heterologous RNA from recombinant Rhabdoviridae.

FIG. 10: Exemplary heterologous RNA. (A) NFKBIA gene RNA target. (B) Influenza virus nucleoprotein gene RNA target. (C) EGFR gene RNA target. (D) KRAS gene RNA target. (E) ELANE gene RNA target. (F) Shigella flexneri hepA gene RNA target. (G) SARS coronavirus nucleoprotein gene RNA target.

FIG. 11: Exemplary heterologous RNA. (A) Influenza virus nucleoprotein gene RNA target, effector RNA as a classical lariat. (B) Influenza virus nucleoprotein gene RNA target, effector RNA as a passenger strand delivery lariat. (C) Influenza virus nucleoprotein gene RNA target, effector RNA as a nuclear sponge. (D) Influenza virus nucleoprotein gene RNA target, ribozyme liberated effector RNA. (E) Exemplary genome of single-stranded, negative sense RNA virus. (F) Exemplary genome of single-stranded, positive sense RNA virus.

FIG. 12: Schematic representation of generation of heterologous RNA from recombinant double-stranded RNA viruses. Abbreviations are as follows: L, M, and S are viral genes; IRES represents an internal ribosome entry site. Reovirus (family: Reoviridae) is used as an exemplary double-stranded RNA virus from which heterologous RNA can be generated.

FIG. 13: Northern blot of exportin-5-positive 293 fibroblasts, exportin-5-negative 293 fibroblasts, dicer-positive immortalized murine fibroblasts, and dicer-negative immortalized murine fibroblasts infected with a mock control, Sindbis-124, or a Sindbis virus encoding a scrambled (scbl) RNA locus. Abbreviations are as follows: m represents mock-infected; s represents Sindbis (scbl) infected; 124 represents Sindbis (mir-124) infected. Lanes 1-3: dicer-positive cells. Lanes 4-6: dicer-negative cells. Lanes 7-9: exportin-5-positive cells. Lanes 10-12: exportin-5-negative cells.

FIG. 14: Classification of certain families of viruses and their structural characteristics. FIG. 14 is a modified figure from Flint et al., Principles of Virology: Molecular Biology, Pathogenesis and Control of Animal Virus. 2nd edition, ASM Press, 2003. A subset of viruses encompassed herein are shown.

FIG. 15: Schematic representation of a microRNA precursor. The 5′ and 3′ ends of the RNA strands are depicted. The individual sections, labeled 1 to 5, are: 1: miRNA frame; 2: passenger strand (sense strand or miRNA star); 3: central miRNA frame (loop); 4: mature miRNA (antisense- or guide strand); and 5: 3′ miRNA frame. The parallel lines indicate hybridized RNA strands.

FIG. 16: (A) Murine embryonic fibroblasts derived from wildtype (WT), Dicer- (Dcr1−/−), DGCR8- (Dgcr8−/−), or IFN-I (Ifnar1−/−)-deficient mice were mock treated or infected with wild-type Sindbis virus (SV) or miR-124-expressing Sindbis virus (SV124) for 24 hours (MOI of 2). The top three panels depict Northern blots probed for miR-124, miR-93, and U6. The bottom two panels represent Western blots for Sindbis virus core protein and actin. (B) Human fibroblasts transfected with a miR-124 targeted GFP plasmid (GFP_miR-124t) were additionally transfected with a miR-124 producing plasmid (p124) or infected with SV or SV124 for 24 hours (MOI of 2). The top three panels depict Western blots for green fluorescent protein (GFP), Sindbis virus core protein and actin. The bottom three panels represent Northern blots probed for miR-124, miR-93, and U6.

FIG. 17: (A) Depiction of a commercially available short interfering RNA (siRNA) generated against human STAT1. (B) Depiction of how the miR-124 hairpin can be modulated to produce the same siRNA. (C) Northern blot analysis of cells mock transfected (−) or transfected with the STAT1 siRNA or STAT1 amiRNA. The Northern blots were probed for STAT1 siRNA and U6. (D) Western blot of cells expressing the amiRNA or wild type miRNA-124 expressing plasmid in the absence of presence of type I interferon (IFN-I). The Western blots were probed for STAT1 and beta-actin.

FIG. 18: Cytoplasmic-mediated miRNA biogenesis. (A) Northern blot of murine embryonic fibroblasts infected with WT or miR-124-expressing SV, vesicular stomatitis virus (VSV), or Influenza A virus (IAV). RNA was probed for miR-124 (top), miR-93 (middle) and U6 (bottom). (B) Human fibroblasts expressing: green fluorescent protein (GFP), an RNA polymerase II-dependent miR-124 plasmid (p124), or miR-124 under the transcriptional control of the T7 polymerase (T7miR-124). Cells transfected with T7_{—124} were additionally transfected with vector (−) or the T7 polymerase (T7 Pol) with and without Sindbis virus infection. Virus infections were performed 6 hours post transfection at an MOI of 1. Samples analyzed as described in (A).

FIG. 19: Model of miR-124 expressing RNA virus constructs. (A) Schematic of miR-124 insertion into IAV between NS1 and NS2 (top), into SV (middle) and into VSV between G and L (middle) and miR-124 driven by cytoplasmic T7 polymerase (bottom). (B) qRT-PCR for viral transcript (SV nsp1, VSV G, and IAV PB2) from samples generated in FIG. 18A. (C) BHK cells were infected with WT or miR-124 expressing SV, VSV and IAV and miR-124 expression was compared to plasmid derived miR-124 (p124). RNA samples probed by small Northern analysis for miR-124 (top) and miR-93 (bottom).

FIG. 20: RNA virus derived production of miRNA in vivo. (A) IFNαR^{-/-} mice were infected with SV, VSV or IAV expressing miR-124. RNA was extracted from the lungs on day 1 post-infection (p.i.) for VSV and day 2 p.i. for SV and IAV and small RNA Northern blots probed for miR-124 (top), miR-93 (middle) and U6 (bottom).

FIG. 21: Cytoplasmic derived miRNA leads to accumulation of star strand. (A) Deep sequencing analysis for miR-124 and miR-124 star strand abundance in mock infected and SV124, VSV124 and IAV124 infected murine embryonic fibroblasts. The Y-axis of each panel depicts the percent of total cellular miRNAs (B) Deep sequencing results for miR-124 (top right side) and miR-124* (top left side). Specific reads from brain, SV124, VSV124, and IAV124 are depicted as percent of total pri-miR-124-2 reads. Reads less than 0.1% are not listed (−). Bottom: Sequence of miR-124-2 with binding interactions beneath responsible for hairpin formation.

FIG. 22: Accumulation of miR-124 star strand from engineered cytoplasmic viruses. Murine embryonic fibroblasts were infected with SV124, VSV124 and IAV124 (at MOI 1, 1, 3 respectively) and 16 hours post-infection RNA was probed by small RNA Northern for miR-124 (A, top), miR-124 star (B, top) and miR-93 (A, B bottom).

FIG. 23: (A) 293 cells transfected with Flag tagged Ago2 or GFP constructs were infected with miR-124 expressing viruses or transfected with p124 as a positive control. RNA from immunoprecipitated Ago2 and GFP, as well as 10% input protein as a loading control, was probed by small Northern blot analysis for miR-124 (top), miR-124 and miR-93 (middle) and U6 (bottom). (B) BHK cells transfected with constructs containing renilla and luciferase encoding scp1 in the 3′UTR which contains endogenous miR-124 target sites. 4 hours post transfection, cells were infected with either WT or miR-124 engineered SV, VSV and IAV at MOI of 1,1 and 3 respectively and the level of luciferase activity determined 12 hours post-infection. Luciferase activity was normalized to untargeted renilla and knock down was measured as decreases compared to WT virus infection. p values for luciferase knockdown are as follows: p124 p=0.0000047, SV124 p=0.0022, VSV124 p=0.00023, and IAV p=0.0849. (C) BHK cells were transfected with GFP containing tandem perfect target sites for miR-124 in the 3′UTR (GFP_{—}124t-3′UTR) and either co-transfected with a plasmid expressing miR-124 (p124) or infected with miR-124 engineered SV, VSV and IAV at MOI of 1, 3, 5 respectively 2 hours post transfection. Protein was isolated 12 hours post-infection and probed by Western blot analysis for GFP (top); SV core, VSV G and IAV NEP virus proteins (middle 3 panels); and B-actin (bottom).

FIG. 24: Protein samples generated in FIG. 23A were analyzed by Western blot analysis for expression of Flag, GFP, SV capsid, VSV G, IAV NP and actin as a loading control.

FIG. 25: (A) Murine embryonic fibroblasts were cultured in the presence or absence of serum for 24 hours and then infected with SV at an MOI of 1 or SV124 at an MOI of 5. Sixteen hours post-infection RNA was extracted and small Northern probed for miR-122 (top), miR-93 (middle) and U6 (bottom). (B) 293 cells were infected with SV or SV124 at an MOI of 1 for 16 hours. RNA was extracted from mock and infected cells as well as huh7 liver cells as a positive control. Small RNA Northern probed for miR-124 (top), miR-93 (middle) and U6 (bottom).

FIG. 26: (A) Murine embryonic fibroblasts were incubated with 10 uM CFSE and cultured with (Right, Mock) or without (Left, Serum Starved)) 10% serum and at 24 and 48 post serum starvation cells were fixed and analyzed by FACS. (B) qRT-PCR for viral transcript from samples generated in FIG. 25A.

FIG. 27: SV124 replication in cytoplasmic miRNA biogenesis deficient cells. (A) qRT-PCR for viral transcript from WT and Dicer1−/− samples generated in FIG. 28A. (B) qRT-PCR for viral transcript from WT and Tarbp−/− samples generated in FIG. 28B. (C) qRT-PCR for viral transcript from WT and PACT−/−samples generated in FIG. 28C. (D) qRT-PCR for viral transcript from WT Ago2−/− samples generated in FIG. 28D.

FIG. 28: (A) Murine embryonic fibroblasts derived from WT (WT MEF) or Dicer deficient (Dicer1^{-/-}) mice mock treated or infected (MOI=1) with SV or SV124 for 24 hours. RNA was extracted for small RNA Northern blot and probed for miR-124 (top), miR-93 (middle), and U6 (bottom). (B) WT (WT MEF) or Tarbp2 deficient (Tarbp2−/−) murine embryonic fibroblasts were mock treated or infected (MOI=1) with SV or SV124 for 24 hours and samples were analyzed as in (A). (C) WT (WT MEF) or PACT deficient (PACT−/−) cells were infected (MOI=1) with SV or SV124 for 24 hours and samples were analyzed as in (A). (D) WT (WT MEF) or Ago2 deficient (Ago2−/−) murine embryonic fibroblasts were mock treated or infected (MOI=1) with SV or SV124 for 24 hours and samples were analyzed as in (A).

FIG. 29: (A) Dgcr8fl/fl murine embryonic stem cells were infected with recombinant Adenovirus expressing either GFP or GFP_Cre (AdV_GFP and AdV_Cre, respectively) at an MOI of 300. Five days post AdV infection, cells were either mock treated or infected with SV or SV124 (MOI=1) for 24 hours and subsequently analyzed via small RNA Northern blot probed for miR-124 (top), miR-93 (middle) and U6 (bottom). (B) Rnasen^{fl/fl} murine embryonic fibroblasts were infected with AdV_GFP or AdV_Cre (MOI=500) for 5 days. Cells were then mock treated or infected with either SV or SV124 (MOI=1) for 24 hours. RNA was analyzed via small RNA Northern blot probed for miR-124 (top), miR-93 (middle) and U6 (bottom). (C) Murine embryonic fibroblasts were mock treated or infected with SV or SV124 (MOI=1) for 0, 2, 4, 8, 12, or 24 hours. Rnasen^{fl/fl} fibroblasts were also infected with AdV_GFP and AdV_Cre (MOI=500) for 5 days and subsequently infected with SV124 at an MOI of 3. RNA was subsequently analyzed 24 hours post-infection via small RNA Northern blot probed for miR-124 (top), miR-93 (middle), and U6 (bottom). (D) pri-miR-124 and mature miR124 bands from (C) were analyzed by densitometry. Dotted line represents limit of detection for mature miR-124.

FIG. 30: SV124 replication in nuclear microprocessor-deficient cells. (A) qRT-PCR for viral transcript from DGCR8^{fl/fl} samples generated in FIG. 29A. (B) qRT-PCR for viral transcript from Rnasen^{fl/fl} samples generated in FIG. 29B.

FIG. 31: In vivo kinetics of IAV-derived miR-124. Balb/C wild type mice were infected intranasally with 1×10⁴ plaque forming units of a control influenza A virus (IAV CTRL) or IAV expressing miR-124 (IAV 124) and whole lung was harvested at 1, 3, or 5 days post infection. Total RNA was analyzed by small Northern blot for virus derived miR-124 and miR-93 expression.

FIG. 32: Sytemic delivery of VSV-derived miR-124. Balb/C wild type mice were infected intranasally with 1×10⁴ plaque forming units of a control Vesicular Stomatitis Virus (VSV CTRL) or VSV expressing miR-124. Heart, Spleen, and Liver were analyzed at 2 days post infection by small Northern blot on total RNA. Northern blot depicts virus-derived miR-124 and endogenous miR-93.

FIG. 33: (A) Multi-cycle growth curve of SV and SV124 performed in wildtype murine fibroblasts (WT), or fibroblasts lacking either Dicer (Dcr1^{-/-}) or a functional IFN-I receptor (Ifnar1^{-/-}). Cells were infected at an MOI of 0.1 and plagued at the indicated time points. p-values of the difference between SV and SV124 replication levels in WT, Dcr1−/−, and Ifnar1−/− at 48 hours post-infection are 0.008, 0.164, and 0.015, respectively. (B) Human fibroblasts were mock treated or transfected with vector or miR-124 producing plasmid (p124). 24 hours post transfection, cells were infected with SV or SV124 (MOI of 2) and harvested 24 hours post-infection. The top two panels depict Western blots for Sindbis virus core protein and actin. The bottom three panels represent Northern blots probed for miR-124, miR-93 and U6. (C) Schematic of miR-124 targeting of the SV124 genome (top) or the SV124 negative strand genome.

### 5. DETAILED DESCRIPTION

Described herein are methods and compositions for the delivery of an effector RNA to a patient. In particular, described herein are methods and compositions for the delivery of an effector RNA that interferes with the expression of a specific target gene(s) in a patient. A target gene can be a gene of a pathogen, or a disease promoting gene (e.g., an oncogene). Target genes and diseases which can be treated by targeting such genes are set forth in Section 5.7, below. Such effector RNAs can be miRNA, mirtrons, shRNA, siRNA, piRNA, svRNA, and antisense RNA.

In one aspect, described herein are recombinant RNA viruses for the delivery of an effector RNA to a subject/patient. Such recombinant RNA viruses comprise a heterologous RNA, which in a host cell, is transcribed, and optionally processed, to give rise to the effector RNA, which in turn can interfere with the expression of a target gene. Recombinant RNA viruses described herein can be derived from RNA viruses. RNA viruses that can be used in the presently described methods and compositions are segmented, single-stranded, negative sense RNA viruses (e.g., Orthomyxoviruses); non-segmented, single-stranded, negative sense RNA viruses (Mononegavirales); non-segmented, single-stranded, positive sense RNA viruses (e.g., Coronaviruses); ambisense RNA viruses (e.g., Bunyavirus and Arenavirus); and double-stranded RNA viruses (e.g., Reoviruses).

In certain embodiments, the recombinant RNA virus is derived from an RNA virus with an RNA genome that is not a retrovirus. In certain embodiments, the recombinant RNA virus is derived from a segmented, single-stranded, negative sense RNA viruses; a non-segmented, single-stranded, negative sense RNA virus; a non-segmented, single-stranded, positive sense RNA viruses; or a double-stranded RNA viruses (e.g., Reoviruses).

In another aspect, described herein are nucleic acids, in particular DNA molecules, encoding a viral genome or a viral genomic segment that include a heterologous RNA as described below.

In another aspect, the recombinant RNA viruses for the delivery of an effector RNA to a subject/patient described herein can be engineered to include a miRNA response element (MRE) and the effector RNA (see, e.g., Perez et al., 2009, Nature Biotechnology 27:572-576; and WO2010101663). Incorporation of an MRE into the viral vector can serve multiple purposes. First, incorporation of an MRE that is responsive to the effector RNA expressed by the virus into a recombinant virus described herein can serve to regulate the virus itself (i.e., a self-regulatory purpose). This can be accomplished by inserting into the viral genome an MRE that is responsive to the effector RNA expressed by the virus such that in the presence of the miRNA to which the MRE is associated (e.g., due to production of the effector RNA by the virus), the virus is attenuated. Tissue-specific miRNA expression has been described (see, e.g., Landgraf et al., 2007, Cell 129(7):1401-1414). As such, a second purpose that can be served by incorporation of an MRE into a recombinant virus described herein is to regulate the ability of the virus to target certain tissues. This can be accomplished by inserting into the viral genome an MRE that is responsive to endogenous miRNA of the subject, wherein said endogenous miRNA of the subject is tissue-specific. As such, the virus will only be able to propagate in certain tissues of the subject, namely those that do not express the miRNA that is specific to the MRE. Such incorporation of MREs can thus serve to regulate the viral vector in the subject, e.g., by attenuating the virus when desirable or warranted by the circumstances, as well as to regulate the virus' tissue-specific miRNA expression. In addition, viruses with known tropisms can be engineered to possess MRE-based regulation of the tissue-specific expression of effector RNA produced by the viruses so as to enhance the existing tropism of the virus. That is, viruses with enhanced tissue targeting can be generated by selecting MREs that result in tissue specific targeting, wherein the tissue targeted is already one which the virus has a natural tropism for.

5.1 Segmented, Single-Stranded, Negative Sense Recombinant RNA Viruses

5.1.1 Chimeric Viral Genomic Segments

In certain embodiments, a heterologous RNA is included in a viral genomic segment of a segmented, single-stranded, negative sense RNA virus, e.g., an orthomyxovirus. Such a viral genomic segment that comprises the heterologous RNA, wherein the recombinant RNA virus is derived from a segmented single-stranded negative sense RNA virus, is referred to in this section as chimeric viral genomic segment. Also described herein are nucleic acids, such as DNA molecules, that encode a chimeric viral genomic segment.

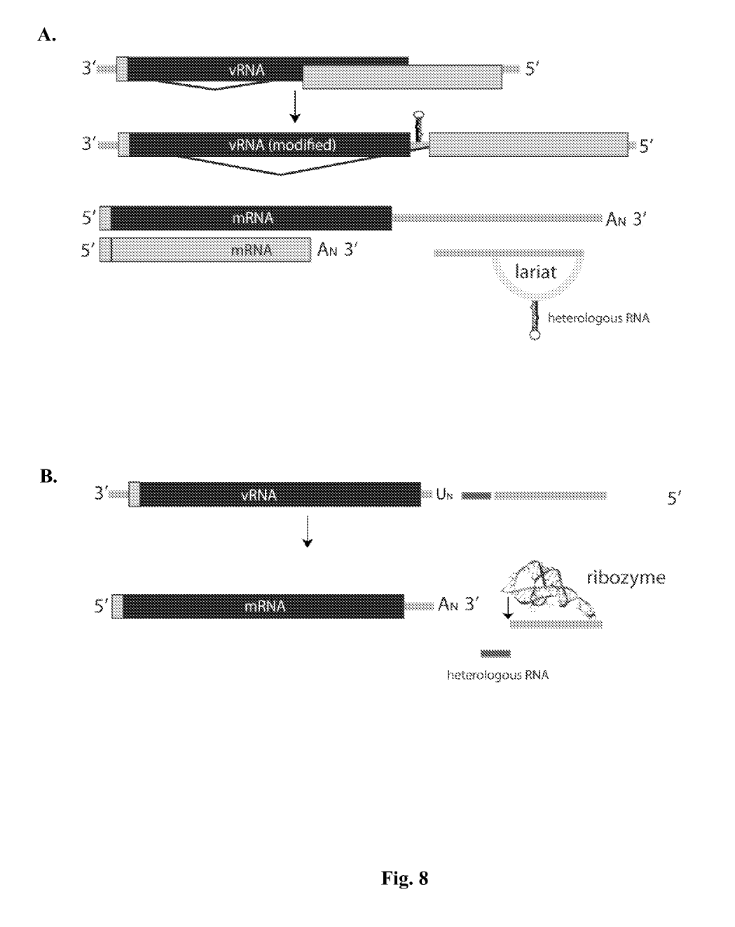

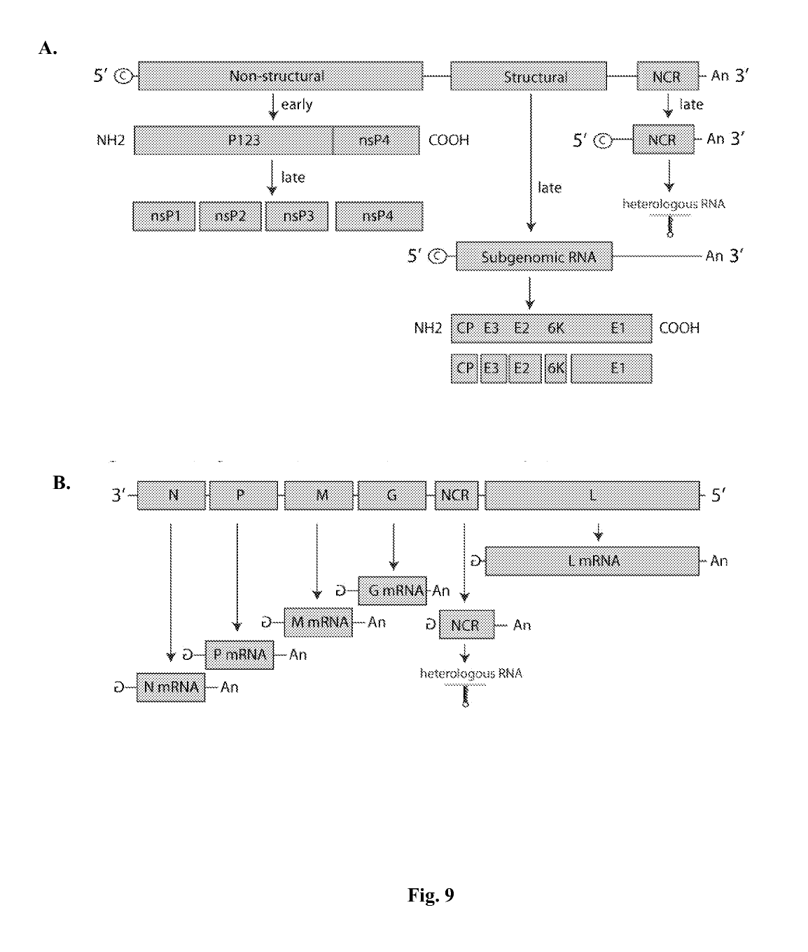

In one aspect, splicing is used to liberate the heterologous RNA from a viral transcript transcribed from a chimeric viral genomic segment. In more specific embodiments, the heterologous RNA is included in a viral segment that naturally undergoes splicing, such as the M1/M2 segment of influenza virus or the NS1/NEP segment of influenza virus. In certain embodiments, the endogenous splice acceptor site is disrupted and recreated after the stop codon of the first open reading frame of the viral segment (e.g., if influenza virus is used, after the stop codon for NS1 if the NS1/NEP segment is used, or after the stop codon of M1 if M1/M2 segment is used), the sequence from the original splice acceptor site to the site of the new splice acceptor site is duplicated after the new splice acceptor site, as illustrated in FIG. 8A, thereby creating an intergenic region and a second open reading frame that is located 5′ of the splice acceptor site (while the first open reading frame is maintained). The heterologous RNA can be cloned into that intergenic region. Without being bound by theory, upon transcription and splicing of the chimeric viral genomic segment, a lariat is formed that includes the heterologous RNA. Also described herein is a DNA molecule that encodes such a chimeric viral genomic segment.

In certain specific embodiments, the endogenous splice acceptor site is disrupted without resulting in an amino acid substitution at that position. In certain specific embodiments, the disruption of the endogenous splice acceptor site results in a conservative amino acid substitution at that position. In certain embodiments, the nucleotides of the splice acceptor site are deleted without creating a frameshift.

Thus, in certain embodiments, provided herein is a chimeric viral genomic segment comprising: (a) packaging signals found in the 3′ non-coding region of an orthomyxovirus gene segment; (b) a first open reading frame of an orthomyxovirus gene that includes a splice donor site; (c) an intergenic region with a heterologous RNA sequence; (d) a splice acceptor site; (e) a second open reading frame of the orthomyxovirus gene; and (f) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s). In a specific embodiment, the chimeric viral genomic segment or the DNA molecule that encodes the chimeric viral genomic segment is isolated.

Influenza virus gene segment packaging signals are known. In addition, techniques for identifying orthomyxovirus gene segment packaging signals are well known. Illustrative packaging assays include the packaging assay disclosed in Liang et al., 2005, J Virol 79:10348-10355 and the packaging assay disclosed in Muramoto et al., 2006, J Virol 80:2318-2325. The description of the packaging assays described in Liang et al. and Muramoto et al. are incorporated herein by reference. Several parameters of the protocols of Liang and Muramoto can be modified; for example various host cells can be used and various reporter genes can be used.

In another aspect, a splice acceptor site and splice donor site can be introduced into an open reading frame (ORF) of viral genomic segment. The creation of the splice acceptor and splice donor sites permits the introduction of an intergenic region and when the intergenic region is spliced out, a lariat is formed. In certain specific embodiments, a sequence in the ORF that is similar to a splice acceptor or splice donor site, respectively, is modified to a splice acceptor site or a splice donor site, respectively, so that the substitutions in the sequence that forms the splice acceptor or splice donor site, respectively, result in fewer amino acid changes. In certain embodiments, any amino acid substitutions that are created by the introduction of the splice acceptor site and the splice donor site are conservative amino acid substitutions. In certain embodiments, the splice acceptor site and the splice donor site are introduced in a portion of the gene that are non-essential for the gene's function or the function of its gene product. In certain embodiments, the introduction of the splice acceptor site and the splice donor site attenuate the virus.

In certain embodiments, provided herein is a chimeric viral genomic segment comprising: (a) packaging signals found in the 3′ non-coding region of an orthomyxovirus gene segment; (b) a first nucleotide sequence that forms part of an open reading frame of an orthomyxovirus gene; (c) a splice donor site; (d) a heterologous RNA sequence; (e) a splice acceptor site; (f) a second nucleotide sequence that forms part of the open reading frame of the orthomyxovirus gene; and (g) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s). In a specific embodiment, the chimeric viral genomic segment or the DNA molecule that encodes the chimeric viral genomic segment is isolated.

In another aspect, the heterologous RNA is included in a chimeric viral genomic segment that naturally does not undergo splicing, such as the PB2, PB1, PA, HA, NP, and NA segments of influenza virus. A splice donor site and a splice acceptor site can be introduced in an untranslated region of the chimeric viral genomic segment. A heterologous RNA can be introduced between the splice donor site and the splice acceptor site such that, upon transcription and splicing, the heterogous RNA is liberated from the viral mRNA.

Thus, in certain embodiments, provided herein is a chimeric viral genomic segment comprising: (a) packaging signals found in the 3′ non-coding region of an orthomyxovirus gene segment; (b) an open reading frame of an orthomyxovirus gene; (c) a splice donor site; (d) a heterologous RNA sequence; (e) a splice acceptor site; and (f) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s). In a specific embodiment, the chimeric viral genomic segment or the DNA molecule that encodes the chimeric viral genomic segment is isolated.

In certain embodiments, provided herein is also a chimeric viral genomic segment comprising: (a) packaging signals found in the 3′ non-coding region of an orthomyxovirus gene segment; (b) a splice donor site; (c) a heterologous RNA sequence; (d) a splice acceptor site; (e) an open reading frame of an orthomyxovirus gene; and (f) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s). In a specific embodiment, the chimeric viral genomic segment or the DNA molecule that encodes the chimeric viral genomic segment is isolated.

In even other aspects, a ribozyme can be used to liberate the heterologous RNA from the viral transcript transcribed from a chimeric viral genomic segment. In such aspects, the heterologous RNA can be flanked by two ribozyme recognition motifs and two self-cleaving ribozymes such that the heterologous RNA is cut out by virtue of the two flanking ribozymes. The heterologous RNA can be located in the 5′ or 3′ untranslated region of the viral transcript transcribed from the chimeric viral genomic segment. If the heterologous RNA is located 3′ of the open reading frame in the viral mRNA, a stretch of greater than ten uracil bases is introduced 3′ of the open reading frame in the mRNA. Self-cleaving RNAs that can be used include, but are not limited to, hammerhead RNA, hepatitis delta virus (HDV) ribozyme, cytoplasmic polyadenylation element binding protein (CPEB3) ribozyme, Ribonuclease P(RNaseP), and the beta-globin co-transcriptional cleavage (CotC) ribozyme.

In certain embodiments, splicing is combined with a ribozyme. Accordingly, a chimeric viral genomic segment can comprise: (a) packaging signals found in the 3′ non-coding region of an orthomyxovirus gene segment; (b) a first nucleotide sequence that forms the open reading frame of an orthomyxovirus gene; (c) a stretch of greater than ten uracil bases; (d) a splice donor site; (e) a heterologous RNA sequence; (e) a ribozyme recognition motif; (f) a self-catalytic RNA (e.g. Hepatitis delta ribozyme); (g) a splice acceptor site; and (h) packaging signals found in the 5′ non-coding region of an orthomyxovirus gene segment. Without being bound by theory, upon transcription and splicing, the resulting lariat that comprises the ribozyme and the heterologous RNA is cleaved to liberate the heterologous RNA from the lariat (see FIG. 8B). Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s).

In certain embodiments, provided herein is a chimeric viral genomic segment comprising: (a) packaging signals found in the 3′ non-coding region of an orthomyxovirus gene segment; (b) a first nucleotide sequence that forms part of an open reading frame of an orthomyxovirus gene; (c) a ribozyme recognition motif; (d) a heterologous RNA sequence; (e) a self-catalytic RNA (e.g. Hepatitis delta ribozyme); (f) a second nucleotide sequence that forms part of the open reading frame of the orthomyxovirus gene; and (g) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s). In a specific embodiment, the chimeric viral genomic segment or the DNA molecule that encodes the chimeric viral genomic segment is isolated.

In certain embodiments, the heterologous RNA is located in the 5′ untranslated region of a chimeric viral genomic segment and is liberated by a ribozyme (e.g., FIG. 8B). Accordingly, a chimeric viral genomic segment can comprise: (a) packaging signals found in the 3′ non-coding region of an orthomyxovirus gene segment; (b) a first nucleotide sequence that forms the open reading frame of an orthomyxovirus gene; (c) a stretch of greater than ten uracil bases; (d) a ribozyme recognition motif; (e) a heterologous RNA sequence; (f) a self-catalytic RNA (e.g. Hepatitis delta ribozyme); and (g) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s).

In certain embodiments, the heterologous RNA is located in the 5′ untranslated region of a chimeric viral genomic segment and is liberated by a ribozyme. Accordingly, a chimeric viral genomic segment can comprise: (a) packaging signals found in the 3′ non-coding region of an orthomyxovirus gene segment; (b) a self-catalytic RNA (e.g. Hepatitis delta ribozyme); (c) a heterologous RNA sequence; (d) a ribozyme recognition motif; (e) an open reading frame of an orthomyxovirus gene; and (g) packaging signals found in the 5′ non-coding region of an orthomyxovirus gene segment. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s).

In certain embodiments, the ribozymes and their target sequences are cloned such that they are only active in the viral mRNA but not in the vRNA. Similarly, the splice acceptor and donor sites are introduced such that they are active in the mRNA and not in the vRNA.

In certain embodiments, the segmented, single-stranded, negative sense RNA virus is replicated and transcribed in the host cell nucleus, such as influenza virus.

In certain other embodiments, the segmented, single-stranded, negative sense RNA virus is replicated and transcribed in the cytoplasm of the host cell, such as Bunyavirus. In specific embodiments, if the recombinant RNA virus is a cytoplasmic virus, the virus is constructed such that the heterologous RNA is released through ribozyme activity and not by splicing.

In certain embodiments, the viral genome segment with the heterologous RNA is not itself a substrate for the Drosha ribonuclease or the Dicer ribonuclease. Instead, the splice and/or ribozyme product is a substrate for the Drosha ribonuclease or the Dicer ribonuclease.

In certain aspects, a chimeric viral genomic segment is constructed that comprises (a) packaging signals found in the 3′ non-coding region of an orthomyxovirus gene segment; (b) a heterologous RNA sequence; and (c) packaging signals found in the 5′ non-coding region of an orthomyxovirus gene segment. In certain specific embodiments, the chimeric viral genomic segment comprises no open reading frame. In more specific embodiments, splice sites are introduced to liberate the heterologous RNA from the transcript. In other more specific embodiments, a ribozyme recognition sequence and the ribozyme that cleaves the ribozyme recognition sequence are introduced 3′ or 5′ of the heterologous RNA to liberate the heterologous RNA from the transcript. In other more specific embodiments, a ribozyme recognition sequences and the ribozymes that cleaves the ribozyme recognition sequence are introduced 3′ and 5′ of the heterologous RNA to liberate the heterologous RNA from the transcript. In even other embodiments, splice sites and ribozymes are combined to liberate the heterologous RNA from the transcript. Also described herein is a DNA molecule that encodes such a chimeric viral genomic segment. In certain embodiments, all non-coding regions of a chimeric viral genomic segment are derived from the same strain and/or from the same species and/or from the same type of RNA virus. In certain embodiments, the non-coding regions of a chimeric viral genomic segment are derived from the same strain and/or from the same species and/or from the same type of RNA virus as the cording region(s). In a specific embodiment, the chimeric viral genomic segment or the DNA molecule that encodes the chimeric viral genomic segment is isolated.

5.1.2 Viruses

Non-limiting examples of segmented, negative-sense, single-stranded RNA viruses that can be engineered to contain and express a chimeric viral genomic segment include: orthomyxoviruses (e.g., influenza A virus, influenza B virus, influenza C virus, thogoto virus, and infectious salmon anemia virus), bunyaviruses (e.g., bunyamwera virus, Hantaan virus, Dugbe virus, Rift Valley fever virus, and tomato spotted wilt virus), and arenaviruses (e.g., Lassa virus, Junin virus, Machupo virus, and lymphocytic choriomeningitis virus). The virus can be any type, species, and/or strain of orthomyxoviruses, bunyaviruses, and arenaviruses. In certain specific embodiments, the virus is an influenza virus. Any type, species, and/or strain of influenza virus can be used with the methods and compositions described herein. In particular, any type, subtype, species, and/or strain of influenza virus can be used to generate a recombinant RNA virus for the delivery of an effector RNA.

In a specific embodiment, a virus engineered to contain and express a chimeric viral genomic segment is an influenza A virus. Non-limiting examples of Influenza A viruses include subtype H10N4, subtype H10N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of Influenza A virus include, but are not limited to: A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d'Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/Gent/V230/92 (H1N1); A/sw/Leipzig/145/92 (H3N2); A/sw/Re220/92hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d'Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d'Armor/604/99 (H1N2); A/sw/Cote d'Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d'Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haseltinne/2617/03hp (H1N1); A/sw/Loningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/swNisbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dotlingen/IDT3780/05 (H1N2); A/sw/Dotlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/swNoglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2).

In a specific embodiment, a virus engineered to contain and express a chimeric viral genomic segment is an influenza B virus. Specific examples of Influenza B viruses include strain Aichi/5/88, strain Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos AiresNL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA110 Philippines/2002), strain clinical isolate SA112 Philippines/2002, strain clinical isolate SA113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain Cordoba/VA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/1/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, and strain Rochester/02/2001.

In a specific embodiment, a virus engineered to contain and express a chimeric viral genomic segment is an influenza C virus. Specific examples of Influenza C viruses include strain Aichi/1/81, strain Ann Arbor/1/50, strain Aomori/74, strain California/78, strain England/83, strain Greece/79, strain Hiroshima/246/2000, strain Hiroshima/252/2000, strain Hyogo/1/83, strain Johannesburg/66, strain Kanagawa/1/76, strain Kyoto/1/79, strain Mississippi/80, strain Miyagi/1/97, strain Miyagi/5/2000, strain Miyagi/9/96, strain Nara/2/85, strain NewJersey/76, strain pig/Beijing/115/81, strain Saitama/3/2000), strain Shizuoka/79, strain Yamagata/2/98, strain Yamagata/6/2000, strain Yamagata/9/96, strain BERLIN/1/85, strain ENGLAND/892/8, strain GREAT LAKES/1167/54, strain JJ/50, strain PIG/BEIJING/10/81, strain PIG/BEIJING/439/82), strain TAYLOR/1233/47, and strain C/YAMAGATA/10/81.

In a specific embodiment, the non-segmented negative-sense single-stranded RNA viruses described herein comprises a miRNA response element (MRE) as described in Section 5 (see, e.g., Perez et al., 2009, Nature Biotechnology 27:572-576; and WO2010101663). In a specific embodiment, the non-segmented negative-sense single-stranded RNA virus described herein that comprises a miRNA response element (MRE) is an influenza virus.

5.1.3 Production of Recombinant RNA Virus

Recombinant RNA viruses described herein comprising a chimeric viral genomic segment described in Section 5.1 can be engineered using any technique known to one of skill in the art, including those described in Section 6, infra. Techniques such as reverse genetics and helper-free plasmid rescue can be used to generate recombinant RNA viruses with a chimeric viral genomic segment described in Section 5.1. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety. In specific embodiments, the recombinant RNA viruses are isolated/purified.

The helper-free plasmid technology can also be utilized to engineer recombinant RNA viruses comprising a chimeric viral genomic segment described in Section 5.1. For example, full length cDNAs of viral segments are amplified using PCR with primers that include unique restriction sites, which allow the insertion of the PCR product into a plasmid vector (see, e.g., Flandorfer et al., 2003, J. Virol. 77:9116-9123; and Nakaya et al., 2001, J. Virol. 75:11868-11873; both of which are incorporated herein by reference in their entireties). The plasmid vector is designed to position the PCR product between a truncated human RNA polymerase I promoter and a hepatitis delta virus ribozyme sequence such that an exact negative (vRNA sense) transcript is produced from the polymerase I promoter. Separate plasmid vectors comprising each viral segment or minimal viral segments as well as expression vectors comprising necessary viral proteins required for replication of the virus are transfected into cells leading to production of recombinant viral particles. For a detailed description of helper-free plasmid technology see, e.g., International Publication No. WO 01/04333; U.S. Pat. No. 6,649,372; Fodor et al., 1999, J. Virol. 73:9679-9682; Hoffmann et al., 2000, Proc. Natl. Acad. Sci. USA 97:6108-6113; and Neumann et al., 1999, Proc. Natl. Acad. Sci. USA 96:9345-9350, which are incorporated herein by reference in their entireties.

In certain embodiments, a recombinant RNA virus is rescued in a cell that is engineered to express the viral proteins necessary to rescue the virus. In certain embodiments, a bidirectional transcription system is used to rescue a recombinant RNA virus (see, e.g., Hoffmann et al. 2002, PNAS 99:11411-11416). In certain embodiments, Vero cells or MDCK are used for the rescue.

Recombinant RNA viruses with a genome comprising a chimeric viral genomic segment described in Section 5.1 can be propagated in any substrate that allows the recombinant RNA virus to grow to titers that permit the isolation of the recombinant RNA virus. For example, the recombinant RNA viruses may be grown in cells (e.g. avian cells, chicken cells (e.g., primary chick embryo cells or chick kidney cells), Vero cells, MDCK cells, human respiratory epithelial cells (e.g., A549 cells), calf kidney cells, mink lung cells, etc.) that are susceptible to infection by the recombinant RNA virus, embryonated eggs or animals (e.g., birds). The recombinant RNA viruses may be recovered from cell culture and separated from cellular components, typically by well known clarification procedures, e.g., such as gradient centrifugation and column chromatography, and may be further purified as desired using procedures well known to those skilled in the art, e.g., plaque assays.

In certain embodiments, a recombinant RNA virus that contains and expresses a chimeric viral genomic segment is attenuated. In certain embodiments, attenuated RNA viruses can be used to engineer recombinant RNA viruses that contain and express a chimeric viral genomic segment. In certain embodiments, the introduction of the heterologous RNA attenuates the RNA virus. In certain embodiments, the heterologous RNA targets a gene of the recombinant RNA viruse thereby attenuating the recombinant RNA viruse. In certain embodiments, the attenuated virus is a cold-adapted attenuated strain, naturally occurring or genetically engineered attenuated strain of viruses carrying a deletion, truncation, or modification of a viral gene, such as, in the case of influenza: PB2, PB1, PA, HA, NP, NA, M1, M2, NS1, NEP, or PB1-F2. Such an attenuated virus is engineered to include a heterologous RNA to create a recombinant RNA virus. In certain embodiments, a virus is engineered to include a heterologous RNA to create a recombinant RNA virus, which is then further genetically modified to attenuate the virus.

In certain embodiments, the recombinant RNA virus is engineered such that the propagation of the virus can be terminated at will. For example, the recombinant RNA virus is a strain of the virus that is known to sensitive to an antiviral agent. If further propagation of the virus in the patient is no longer desired, the antiviral can be administered to discontinue propagation of the virus. In certain embodiments, the virus carries a suicide gene that prevents the virus from undergoing more than a complete life cycle thereby prevent further infection of the patient with the virus.

In certain embodiments, a recombinant RNA virus is engineered such that the virus can undergo only a limited number of replications in the subject. In more specific embodiments, the genome of a recombinant RNA virus is replicated 1, 2, 3, 4, 5, 6, 7, 8, 9, 10, 11, 12, 13, 14, 15, 16, 17, 18, 19, or 20 times in the subject. In other more specific embodiments, the recombinant RNA virus undergoes 1, 2, 3, 4, 5, 6, 7, 8, 9, 10, 11, 12, 13, 14, 15, 16, 17, 18, 19, or 20 times replication cycles in the subject.

In some embodiments, the attenuation can result, in part, from a mutation in a gene required for efficient replication of the recombinant RNA virus. Further, attenuation can result, in part, from a combination of one or more mutations in other viral genes. In certain embodiments, the recombinant RNA virus is an influenza virus that has a truncated or deleted NS1 genes, such as described in issued U.S. Pat. No. 6,468,544, issued Oct. 22, 2002, U.S. Pat. No. 6,866,853, issued Mar. 15, 2005, and U.S. Pat. No. 6,669,943, issued Dec. 30, 2003 and U.S. Pat. No. 7,588,768, issued Sep. 15, 2009. A recombinant RNA virus may also be engineered from natural variants, such as the A/turkey/Ore/71 natural variant of influenza A, or B/201, and B/AWBY-234, which are natural variants of influenza B.

In certain specific embodiments, the recombinant RNA virus is derived from influenza virus and attenuation is accomplished by interfering with an svRNA of influenza virus (see Perez et al., “Influenza A virus-generated small RNAs regulate the switch from transcription to replication,” PNAS, published online on Jun. 1, 2010).

In certain embodiments, a recombinant RNA virus is used that does not normally infect the intended subject. Thus, in a specific embodiment, the intended subject is a human and the recombinant RNA virus is derived from an RNA virus that does not normally infect humans.

In certain embodiments, the replication rate of a segmented negative-sense single-stranded RNA virus that carries a heterologous RNA is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of a segmented negative-sense single-stranded RNA virus that carries a heterologous RNA is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of a segmented negative-sense single-stranded RNA virus that carries a heterologous RNA is between 5% and 20%, between 10% and 40%, between 25% and 50%, between 40% and 75%, between 50% and 80%, or between 75% and 90% of the replication rate of the wild type virus from which the recombinant virus is derived under the same conditions.

5.1.4 Rewiring of Genomic Segments

In certain embodiments, the chimeric viral genomic segment is “rewired” with one or more other viral genomic segments to prevent reassortment-mediated loss of the heterologous RNA-carrying segment (see, Gao & Palese 2009, PNAS 106:15891-15896; and International Application Publication No. WO11/014,645). Specific packaging signals for individual influenza virus RNA segments are located in the 5′ and 3′ noncoding regions as well as in the terminal regions of the ORF of an RNA segment. By placing the packaging sequences of a first viral genomic segment onto the ORF of a second viral genomic segment and mutating the original packaging regions in the ORF of the second segment, a chimeric second segment is created with the packaging identity of the first segment. By the same strategy, the first segment can be engineered to acquire the packaging identity of the second segment. Such a rewired virus can have the packaging signals for all genomic segments, but it does not have the ability to independently reassort the first and the second segment. In a specific embodiment the NS and the HA segments are rewired.

In certain embodiments, the genomic segment that carries the heterologous RNA is rewired with the genomic segment that encodes the protein that is responsible, or mainly responsible, for the tropism of the virus. In certain other embodiments, the genomic segment that carries the heterologous RNA is rewired with the genomic segment that encodes the RNA dependent RNA polymerase of the recombinant RNA virus.

For example, genomic segments can be rewired if the heterologous RNA is, e.g., an svRNA mimetic or anti-svRNA to prevent loss of expression of the segment that carries the heterologous RNA.

5.1.5 Tropism

In certain embodiments, the natural tropism, or modified tropism, of the RNA virus from which the recombinant RNA virus is derived is used to target the effector RNA to a desired cell type, tissue, organ, or body part. Thus, if it is desired to affect expression of a target gene in, e.g., pulmonary tissue, a recombinant RNA virus that infects only pulmonary tissue is used. The viral genomic segment that is responsible for the viral tropism can be different or it can be the same as the chimeric viral genomic segment that carries the heterologous RNA.

In certain embodiments, the viral gene that is responsible for the tropism of the recombinant RNA virus is replaced with a gene from a second virus with a different tropism such that the recombinant RNA virus acquires the tropism of the second virus. For example, HA and/or NA of influenza can be replaced with the G gene of VSV (encoding either the HA and NA packaging sequence), yielding a virus whose entry will not be restricted to any cell (See, Watanabe et al. J. Virol. 77 (19): 10575.). In another specific embodiment, the coding regions of HA and NA can be exchanged with gp41 and gp120, respectively, to obtain a recombinant RNA virus whose tropism would mimic that of HIV. In yet another embodiment, HA and/or NA could be replaced with gpE1 of HCV to obtain a recombinant RNA virus that mimics the tropism of HCV.

In certain embodiments, a recombinant RNA virus comprises a viral genomic segment as described in WO 2007/064802 published on Jun. 6, 2007.

5.2 Non-Segmented Negative-Sense Single-Stranded Recombinant RNA Viruses

5.2.1 Chimeric Viral Genome

In certain embodiments, a recombinant RNA virus is derived from a non-segmented negative-sense single-stranded RNA virus. A heterologous RNA is introduced in the genome of a non-segmented negative-stranded RNA virus. The resulting genome is referred to in this section as chimeric viral genome. In certain embodiments, the transcribed heterologous RNA processed to give rise to an effector RNA. In certain embodiments, the heterologous RNA is an effector RNA.

Insertion of a heterologous RNA into a non-segmented negative-sense single-stranded RNA virus genome can be accomplished by either a complete replacement of a viral coding region with the heterologous RNA, or by a partial replacement of the same, or by adding the heterologous nucleotide sequence to the non-coding region of the viral genome. The resulting genome is referred to as chimeric viral genome. Also described herein are nucleic acids, such as DNA molecules, that encode such a chimeric viral genome.

In certain embodiments, a gene that is not essential from the viral life cycle of the non-segmented negative-sense single-stranded RNA virus is completely or partially replaced with the heterologous RNA.

A heterologous RNA can be added or inserted at various positions of the non-coding region of a viral genome. In certain embodiments, the heterologous RNA is inserted between two genes in the viral genome, i.e., in an intergenic region, the 3′ leader sequence, or the 5′ trailer sequence. In one embodiment, the non-segmented negative-sense single-stranded RNA virus is parainfluenza virus and the heterologous RNA is inserted between the first and the second, the second and the third, the third and the fourth, the fourth and the fifth, or the fifth and the sixth viral gene to be transcribed.

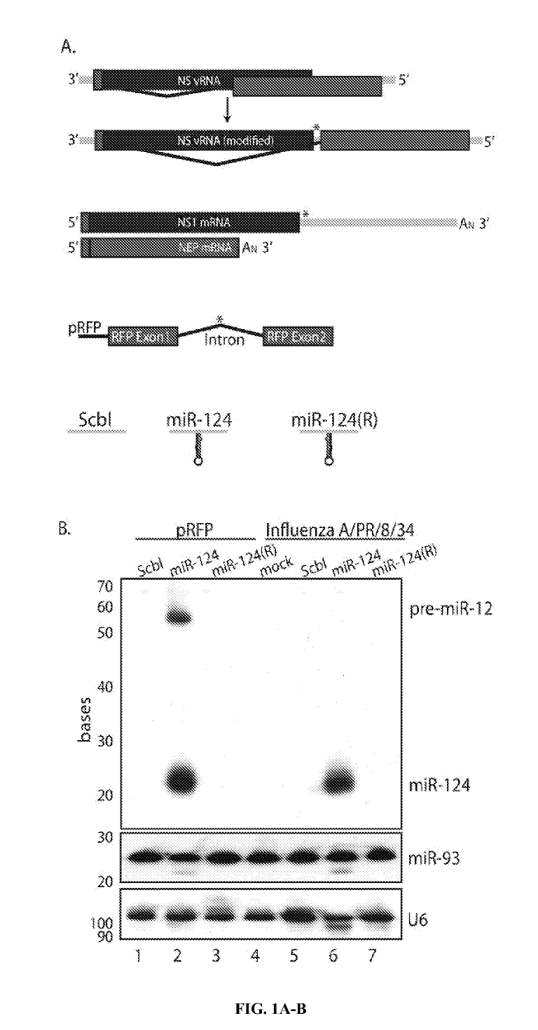

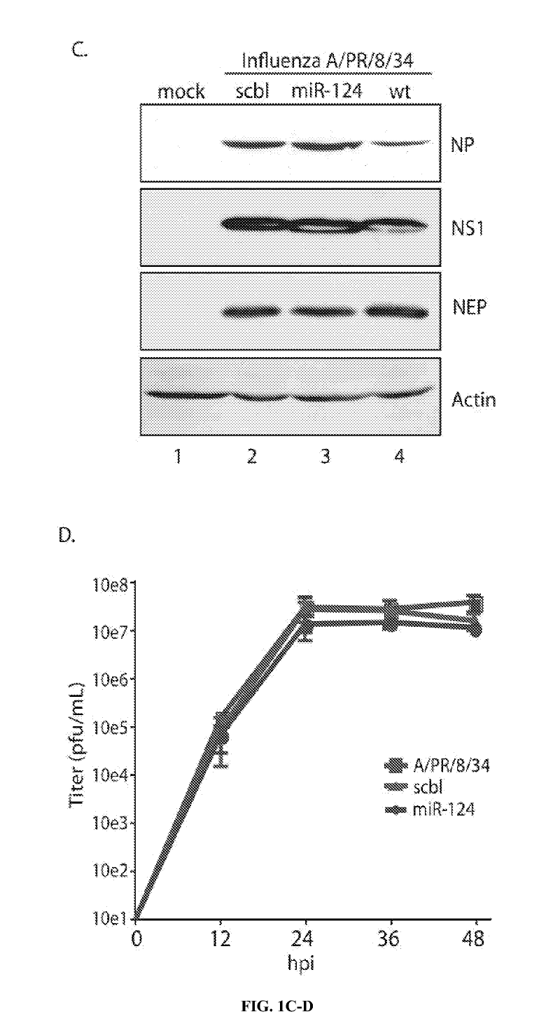

In certain embodiments, the heterologous RNA is flanked by a gene-start on the 3′ end and a gene stop at the 5′ end of a gene of the same non-segmented negative-sense single-stranded RNA virus. For example, if the a non-segmented negative-sense single-stranded RNA virus is respiratory syncytial virus, the gene start and gene stop from the N, P, M, SH, G, F, M2, or L gene or a combination thereof could be used. Illustrative methods for manipulating a non-segmented negative-sense single-stranded RNA virus are described, e.g., in Haller et al. 2003, J Gen Vir 84:2153-2162 (see FIG. 1).

In certain embodiments, a non-segmented negative-sense single-stranded RNA virus is used that has a transcriptional gradient, wherein the genes located at the 3′ end are transcribed at higher levels than the genes located at the 5′ end. Without being bound by theory, inserting the heterologous RNA closer to the 3′ end can result in stronger expression of the heterologous RNA compared to insertion closer to the 5′ end due to a transcriptional gradient that occurs across the genome of the virus. In a specific embodiment, if strong expression of a heterologous RNA is desired, the heterologous RNA is found closer to the 3′ end of the viral genome.

In certain embodiments, a non-segmented negative-sense single-stranded RNA virus that follows the rule of six (i.e., the number of nucleotides of the genome of the virus is a multiple of six for the virus to propagate efficiently) is used to engineer a recombinant RNA virus that contains and expresses a heterologous RNA. If a virus that follows the rule of six is used, the heterologous RNA can be of such length that the recombinant genome of the recombinant non-segmented negative-sense single-stranded RNA virus still follows the rule of six. In other embodiments, the heterologous RNA can be of such length that the recombinant genome of the recombinant RNA virus derived from a non-segmented negative-sense single-stranded RNA virus does not follow the rule of six and the virus is attenuated.

In certain embodiments, a chimeric virus genome comprises: (a) polymerase initiation sites found in the 3′ non-coding region of the genome; (b) any number of viral genes required for viral replication; (c) a heterologous RNA sequence; and (d) any polymerase replication sites found in the 5′ non-coding region of the genome. In certain embodiments, a chimeric virus genome comprises: (a) polymerase initiation sites found in the 3′ non-coding region of the genome; (b) any number of viral genes required for viral replication; (c) a heterologous RNA sequence flanked by ribozyme recognition sequences and one or more ribozymes such that the heterologous RNA is cleaved from the viral transcript; and (d) any polymerase replication sites found in the 5′ non-coding region of the genome. In certain embodiments, a chimeric virus genome comprises: (a) polymerase initiation sites found in the 3′ non-coding region of the genome; (b) any number of viral genes required for viral replication; (c) a ribozyme recognition sequence; (d) a heterologous RNA sequence; and (e) a ribozyme that cleaves the ribozyme recognition sequence is (c). Also described herein are nucleic acids, such as DNA molecules, that encode such a chimeric viral genome. In a specific embodiment, the chimeric virus genome or the DNA molecule that encodes the chimeric virus genome is isolated.

In certain embodiments, a chimeric rhabdoviridae (or paramyxoviridae) genome comprises: (a) polymerase initiation sites found in the 3′ non-coding region of the genome; (b) any number of viral genes required for viral replication; (c) a heterologous RNA sequence whose 5′ and 3′ sequences adhere to the requirements for polymerase initiation and termination; (d) any remaining viral genes required for viral replication; and (e) polymerase replication sites found in the 5′ non-coding region of the genome. Also described herein are nucleic acids, such as DNA molecules, that encode such chimeric viral genomes.

5.2.2 Viruses

Non-limiting examples of non-segmented, negative-sense, single-stranded RNA viruses that can be engineered to contain and express a heterologous RNA include: rhabdoviruses (e.g., vesicular stomatitis virus (VSV), rabies, and rabies-related viruses), paramyxoviruses (e.g., Newcastle Disease Virus (NDV), measles virus, mumps virus, parainfluenza viruses such as Sendai virus, and pneumoviruses such as respiratory syncytial virus (RSV) and metapneumovirus), filoviruses (e.g., Ebola virus and Marburg virus), hepatitis delta virus, and bomaviruses. In a specific embodiment, the non-segmented negative-sense single-stranded RNA virus is a chimeric bovine/human parainfluenza virus type 3 (see, e.g., Tang et al. 2005, Vaccine 23:1657-1667). In another specific embodiment, the non-segmented negative-sense single-stranded RNA virus is a velogenic, mesogenic, or lentogenic strain of NDV. Specific examples of NDV strains include, but are not limited to, the 73-T strain, NDV HUJ strain, Ulster strain, MTH-68 strain, Italien strain, Hickman strain, PV701 strain, Hitchner B1 strain, La Sota strain (see, e.g., Genbank No. AY845400), YG97 strain, MET95 strain, Roakin strain, and F48E9 strain.

5.2.3 Production of Recombinant Viruses

Any method known to the skilled artisan can be used to rescue the virus that carries the heterologous RNA. Reverse genetics can be used to rescue the virus. Helper virus-free rescue can be used. See, e.g., U.S. Patent Application Publication No. 20040142003 published on Jul. 22, 2004. In specific embodiments, the recombinant viruses are isolated/purified.

In certain embodiments, the recombinant RNA virus is modified such that the virus is attenuated in the patient. In specific embodiments, parainfluenza virus is used as to produce the recombinant RNA virus and one or more of the viral genes is mutated to attenuate the virus, namely, the N, P, M, F, HN, or L gene.

In certain embodiments, a recombinant RNA virus is rescued in a cell that is engineered to express the viral proteins necessary to rescue the virus. In certain embodiments, Vero cells or MDCK are used for the rescue. In certain embodiments, eggs are used for viral growth.

In certain embodiments, a recombinant RNA virus is used that does not normally infect the intended subject. For example, a bovine parainfluenza virus, e.g., bovine parainfluenza virus type 3, is attenuated in a human subject. Thus, in a specific embodiment, a recombinant RNA virus is derived from bovine parainfluenza virus where the intended subject is a human.

In certain embodiments, the recombinant RNA virus is engineered such that the propagation of the virus can be terminated at will. For example, the recombinant RNA virus is a strain of the virus that is known to sensitive to an antiviral agent. If further propagation of the virus in th patient is no longer desired, the antiviral can be adminstered to discontinue propagation of the virus. In certain embodiments, the virus carries a suicide gene that prevents the virus from undergoing more than a complete life cycle thereby prevent further infection of the patient with the virus.

In certain embodiments, a recombinant RNA virus is engineered such that the virus can undergo only a limited number of replications in the subject. In more specific embodiments, the genome of a recombinant RNA virus is replicated 1, 2, 3, 4, 5, 6, 7, 8, 9, 10, 11, 12, 13, 14, 15, 16, 17, 18, 19, or 20 times in the subject. In other more specific embodiments, the recombinant RNA virus undergoes 1, 2, 3, 4, 5, 6, 7, 8, 9, 10, 11, 12, 13, 14, 15, 16, 17, 18, 19, or 20 times replication cycles in the subject.

In some embodiments, the attenuation can result, in part, from a mutation in a gene required for efficient replication of the recombinant RNA virus. Further, attenuation can result, in part, from a combination of one or more mutations in other viral genes.

In certain embodiments, the replication rate of a non-segmented negative-sense single-stranded RNA virus that carries a heterologous RNA is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of a non-segmented negative-sense single-stranded RNA virus that carries a heterologous RNA is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of a non-segmented negative-sense single-stranded RNA virus that carries a heterologous RNA is between 5% and 20%, between 10% and 40%, between 25% and 50%, between 40% and 75%, between 50% and 80%, or between 75% and 90% of the replication rate of the wild type virus from which the recombinant virus is derived under the same conditions.

5.2.4 Tropism

In certain embodiments, the natural tropism, or modified tropism, of the RNA virus from which the recombinant RNA virus is derived is used to target the effector RNA to a desired cell type, tissue, organ, or body part. Thus, if it is desired to affect expression of a target gene in, e.g., pulmonary tissue, a recombinant RNA virus that infects only pulmonary tissue is used.

In certain embodiments, the viral gene that is responsible for the tropism of the recombinant RNA virus is replaced with a gene from a second virus with a different tropism such that the recombinant RNA virus acquires the tropism of the second virus. For example, the recombinant RNA virus is not derived from a VSV but the glycoprotein of the recombinant RNA virus has been replaced with the G gene of VSV (encoding either the HA and NA packaging sequence), yielding a virus whose entry will not be restricted to any cell. In another specific embodiment, the coding regions of a glycoprotein of the recombinant RNA virus can be exchanged with gp41 and gp120, respectively, to obtain a recombinant RNA virus whose tropism would mimic that of HIV. In yet another embodiment, a glycoprotein of a recombinant RNA virus could be replaced with gpE1 of HCV to obtain a recombinant RNA virus that mimics the tropism of HCV.

In certain specific embodiments, the glycoprotein of the recombinant RNA virus is replaced with the glycoprotein of Borna Disease Virus to target neural tissue (Bajramovic 2003, J Virol 77:12222-12231).

5.3 Non-Segmented Positive Strand RNA Viruses

5.3.1 Chimeric Viral Genome

In certain embodiments, a non-segmented positive strand RNA virus can be used to engineer a recombinant RNA virus that contains and expresses a heterologous RNA. The resulting recombinant RNA virus has a chimeric viral genome that contains and expresses a heterologous RNA. In certain embodiments, the transcribed heterologous RNA is processed to give rise to an effector RNA. In certain embodiments, the heterologous RNA is an effector RNA.

In certain aspects, the heterologous RNA is introduced into the 3′ untranslated region of the genome of the non-segmented positive strand RNA virus to engineer a chimeric viral genome. Also described herein are nucleic acids, such as DNA molecules, that encode the chimeric viral genome.

In certain aspects, the heterologous RNA is flanked by ribozyme recognition sequences and their respective ribozymes such that the heterologous RNA is liberated from the viral genome via the self-cleaving ribozymes. In more specific embodiments, the ribozymes are active only in the negative sense strand that is produced during the viral life cycle in the host cell. In even more specific embodiments, the ribozymes are not 100% efficient such that a portion of negative sense strand genomes of the virus remain intact.

In certain embodiments, the heterologous RNA is introduced in the 3′ region of a transcribed portion of the genome of the non-segmented positive strand RNA virus. In more specific embodiments, the heterologous RNA is flanked by ribozyme recognition sequences and their respective ribozymes such that the heterologous RNA is liberated from the viral genome via the self-cleaving ribozymes. In more specific embodiments, the ribozymes are active only in the negative sense strand that is produced during the viral life cycle in the host cell. In even more specific embodiments, the ribozymes are not 100% efficient such that a portion of negative sense strand genomes of the virus remain intact.

In certain embodiments, generation of subgenomic RNA is used to liberate the heterologous RNA from the viral genome. An internal transcription start site for the transcription of a subgenomic RNA, i.e., a subgenomic promoter followed by the heterologous RNA is introduced in the 5′ terminal, untranslated region of the genome of the non-segmented positive strand RNA virus. In certain embodiments, a subgenomic mRNA promoter sequence is introduced into a nonessential region of the viral genome. In certain embodiments, the artificially introduced subgenomic mRNA promoter is the most 3′ located subgenomic promoter. In certain embodiments, no translated regions are located 3′ of the artificially introduced subgenomic mRNA promoter.

Without being bound by theory, coronavirus and arterivirus subgenomic RNA transcripts also contain a common 5′ leader sequence, which is derived from the genomic 5′ end (Pasternak 2006, J Gen Virol 87:1403-1421). The assembly between 5′leader and subgenomic RNA transcript, which is located at the 3′ end of the genome, is thought to occur through co-transcriptional fusion (Pasternak 2006, J Gen Virol 87:1403-1421). In certain embodiments, the 5′ portion of the heterologous RNA is introduced into the 5′ leader sequence and the 3′ portion of the heterologous RNA is introduced into the 5′ part of a subgenomic RNA or the 5′ part of an artificial subgenomic RNA with an artificially introduced subgenomic promoter. Upon transcription of the subgenomic RNA, the 5′ leader sequence and the 3′ subgenomic RNA are brought together and the heterologous RNA is united.

In certain embodiments, a chimeric viral genome comprises: (a) a polymerase initiation sites found in the 5′ non-coding region of the genome; (b) the open reading frame for the non-structural viral proteins; (c) the internal recognition sequence for subgenomic RNA synthesis; (d) the open reading frame for the structural viral proteins; (e) a heterlogous RNA sequence; (f) a poly A tail. In certain, more specific, embodiments, a chimeric viral genome comprises: (a) a polymerase initiation sites found in the 5′ non-coding region of the genome; (b) the open reading frame for the non-structural viral proteins; (c) the internal recognition sequence for subgenomic RNA synthesis; (d) the open reading frame for the structural viral proteins; (e) a ribozyme recognition sequence; (f) a heterlogous RNA sequence; (g) a self-cleaving ribozyme that cleaves the ribozyme recognition sequence (see segment (e)); and (h) a poly A tail. In other, more specific, embodiments, a chimeric viral genome comprises: (a) a polymerase initiation sites found in the 5′ non-coding region of the genome; (b) the open reading frame for the non-structural viral proteins; (c) the internal recognition sequence for subgenomic RNA synthesis; (d) the open reading frame for the structural viral proteins; (e) a self-cleaving ribozyme and its ribozyme recognition sequence; (f) a heterlogous RNA sequence; (g) a self-cleaving ribozyme and its ribozyme recognition sequence; and (h) a poly A tail. Also described herein are nucleic acids, such as DNA molecules, encoding such chimeric viral genomes. In a specific embodiment, the chimeric virus genome or the DNA molecule that encodes the chimeric virus genome is isolated.

In certain embodiments, a chimeric togaviridae genome comprises: (a) polymerase initiation sites found in the 5′ non-coding region of the genome; (b) the open reading frame for the non-structural viral proteins; (c) the internal recognition sequence for subgenomic RNA synthesis; (d) the open reading frame for the structural viral proteins; (d) a second internal recognition sequence for subgenomic RNA synthesis; (e) a heterlogous RNA sequence whose 5′ and 3′ sequences adhere to the requirements for polymerase initiation and termination; (f) polymerase replication sites found in the 3′ non-coding region of the genome including the 3′ conserved sequence element (CSE) and the poly A tail. Also described herein are nucleic acids, such as DNA molecules, encoding such a chimeric togaviridae genome. In a specific embodiment, the chimeric virus genome or the DNA molecule that encodes the chimeric virus genome is isolated.

In certain aspects, the heterologous RNA is incorporated into the part of the genome that encodes the nonstructural proteins. See, e.g., Liang and Li 2005, Gene Therapy and Molecular Biology 9:317-323. In more specific embodiments, the heterologous RNA is incorporated into the chimeric viral genome such that the heterologous RNA is located at the 3′ end of the transcript that encodes the nonstructural proteins. In certain specific embodiments, ribozymes are used to liberate the heterologous RNA from the transcript. Also described herein are nucleic acids, such as DNA molecules, encoding such a chimeric togaviridae genome.

5.3.2 Viruses

In certain embodiments, a recombinant RNA virus described herein is derived from one of the following RNA viruses: Picornaviruses, togaviruses (e.g., Sindbis virus), flaviviruses, and coronaviruses. The virus can be any type, species, and/or strain of picornavirus, togavirus (e.g., Sindbis virus), flavivirus, and coronavirus.

For example, if the chimeric viral genome is derived from a Sindbis virus genome, the subgenomic promoter identified in Levis et al. 1990, J Virol 64:1726-1733 can be used (see also, Hahn et al. 1992, PNAS 89:2679-2683).

5.3.3 Production of Recombinant Viruses

Any method known to the skilled artisan can be used to rescue the virus that carries the heterologous RNA. Without being bound by theory, genomic RNA of non-segmented positive strand RNA virus is itself infectious. Thus, helper-virus free rescue can for example be accomplished by introducing a cDNA that encodes the chimeric viral genome into a host cell. In more specific embodiments, the cDNA that encodes the chimeric viral genome is transcribed from a plasmid. In specific embodiments, the recombinant RNA viruses are isolated/purified.

In certain specific embodiments, a noncytopathic Sindbis virus is used to engineer a recombinant RNA virus (Agapov et al. 1998, PNAS 95:12989-12994). In specific embodiments, a chimeric viral genome is derived from a Sindbis virus with a mutation in the nsP2 gene. In more specific embodiments, a chimeric viral genome is derived from a Sindbis virus with a mutation that results in an amino acid substitution at position 726 of the nsP2 protein. In even more specific embodiments, a chimeric viral genome is derived from a Sindbis virus with a mutation that results in a P to L amino acid substitution at position 726 of the nsP2 protein (Agapov et al. 1998, PNAS 95:12989-12994).

In certain embodiments, the recombinant RNA virus is modified such that the virus is attenuated in the intended subject, e.g., a human patient. In specific embodiments, a nonstructural gene or a structural gene is mutated to achieve attenuation. In certain embodiments, a noncoding sequence, e.g., 5′ leader sequence or promoter for an RNA dependent RNA polymerase is mutated to achieve attenuation.

In certain embodiments, a recombinant RNA virus is used that does not normally infect the intended subject. Thus, in a specific embodiment, a recombinant RNA virus is derived from a non-human RNA virus wherein the intended subject is human.

In certain embodiments, the recombinant RNA virus is engineered such that the propagation of the virus can be terminated at will. For example, the recombinant RNA virus is a strain of the virus that is known to sensitive to an antiviral agent. If further propagation of the virus in th patient is no longer desired, the antiviral can be adminstered to discontinue propagation of the virus. In certain embodiments, the virus carries a suicide gene that prevents the virus from undergoing more than a complete life cycle thereby prevent further infection of the patient with the virus.

In certain embodiments, a recombinant RNA virus is engineered such that the virus can undergo only a limited number of replications in the subject. In more specific embodiments, the genome of a recombinant RNA virus is replicated 1, 2, 3, 4, 5, 6, 7, 8, 9, 10, 11, 12, 13, 14, 15, 16, 17, 18, 19, or 20 times in the subject. In other more specific embodiments, the recombinant RNA virus undergoes 1, 2, 3, 4, 5, 6, 7, 8, 9, 10, 11, 12, 13, 14, 15, 16, 17, 18, 19, or 20 times replication cycles in the subject.

In some embodiments, the attenuation can result, in part, from a mutation in a gene required for efficient replication of the recombinant RNA virus. Further, attenuation can result, in part, from a combination of one or more mutations in other viral genes.

In certain embodiments, the replication rate of a non-segmented positive-sense single-stranded RNA virus that carries a heterologous RNA is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of a non-segmented positive-sense single-stranded RNA virus that carries a heterologous RNA is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of a non-segmented positive-sense single-stranded RNA virus that carries a heterologous RNA is between 5% and 20%, between 10% and 40%, between 25% and 50%, between 40% and 75%, between 50% and 80%, or between 75% and 90% of the replication rate of the wild type virus from which the recombinant virus is derived under the same conditions.

5.3.4 Tropism

In certain embodiments, the natural tropism, or modified tropism, of the RNA virus from which the recombinant RNA virus is derived is used to target the effector RNA to a desired cell type, tissue, organ, or body part. Thus, if it is desired to affect expression of a target gene in, e.g., pulmonary tissue, a recombinant RNA virus that infects only pulmonary tissue is used.

In certain embodiments, the viral gene that is responsible for the tropism of the recombinant RNA virus is replaced with a gene from a second virus with a different tropism such that the recombinant RNA virus acquires the tropism of the second virus. In specific embodiments, the second virus is of the same type as the recombinant RNA virus. For example, a glycoprotein of the recombinant RNA virus can be replaced with the G gene of VSV, yielding a virus whose entry will not be restricted to any cell. In another specific embodiment, the coding regions of a glycoprotein of the recombinant RNA virus can be exchanged with gp41 and gp120, respectively, to obtain a recombinant RNA virus whose tropism would mimic that of HIV. In yet another embodiment, a glycoprotein of a recombinant RNA virus could be replaced with gpE1 of HCV to obtain a recombinant RNA virus that mimics the tropism of HCV.

In certain specific embodiments, the glycoprotein of the recombinant RNA virus is replaced with the glycoprotein of Borna Disease Virus to target neural tissue (Bajramovic 2003, J Virol 77:12222-12231).

In certain embodiments, the recombinant RNA virus expresses a soluble receptor (soR)-based expression construct fused to an epidermal growth factor (EGF) receptor targeting moiety to target the recombinant RNA virus to tumor cells (Verheije et al. 2009, J Virol 83:7507-7516). In a specific embodiment, the recombinant RNA virus is derived from a coronavirus and expresses a soluble receptor (soR)-based expression construct fused to an epidermal growth factor (EGF) receptor targeting moiety to target the recombinant RNA virus to tumor cells (Verheije et al. 2009, J Virol 83:7507-7516).

5.4 Double-Stranded RNA Viruses

5.4.1 Chimeric Viral Genomic Segment

In certain embodiments, a double-stranded RNA virus can be used to generate a recombinant RNA virus that contains and expresses a heterologous RNA. A heterologous RNA is introduced into a viral genome segment; the resulting chimeric viral genomic segment contains and expresses a heterologous RNA. In certain embodiments, a heterologous RNA is transcribed and processed to give rise to an effector RNA. In other embodiments, a heterologous RNA, once transcribed, is an effector RNA.

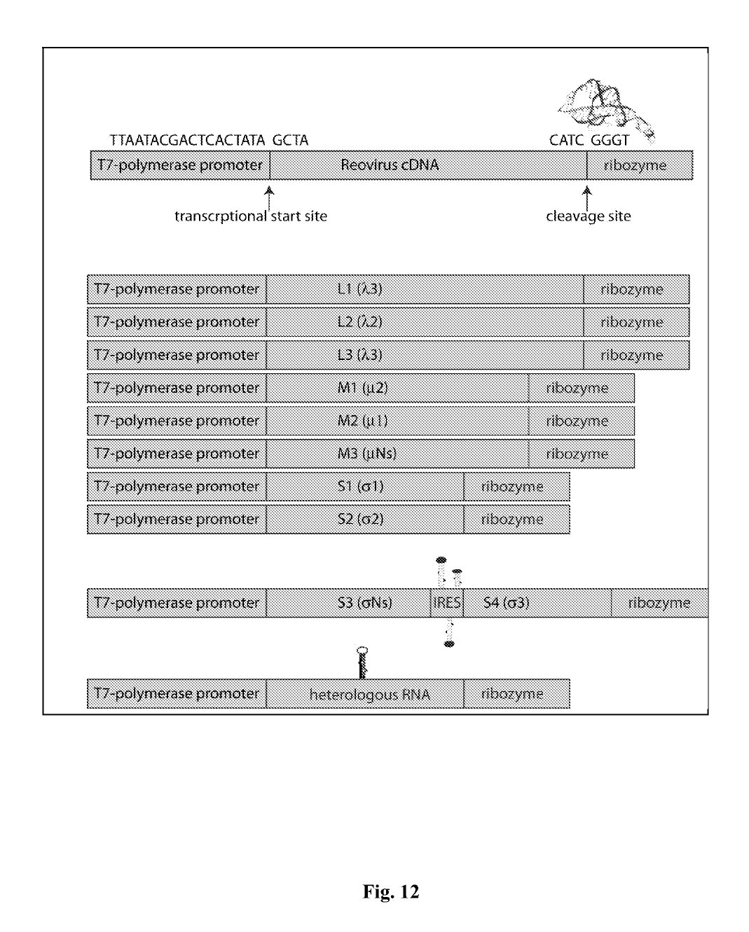

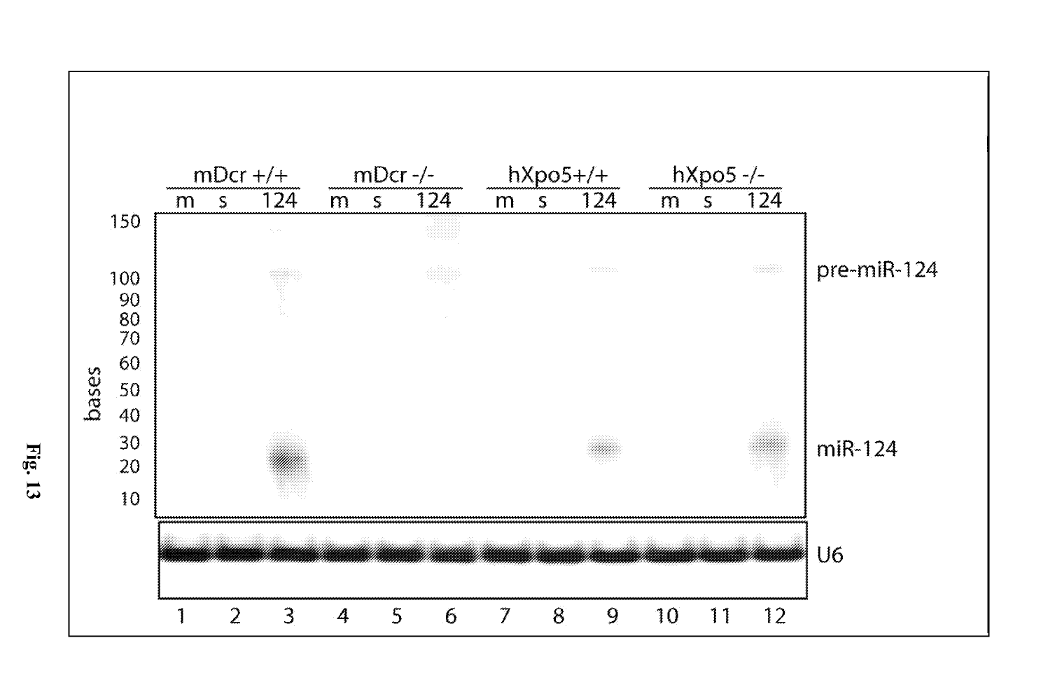

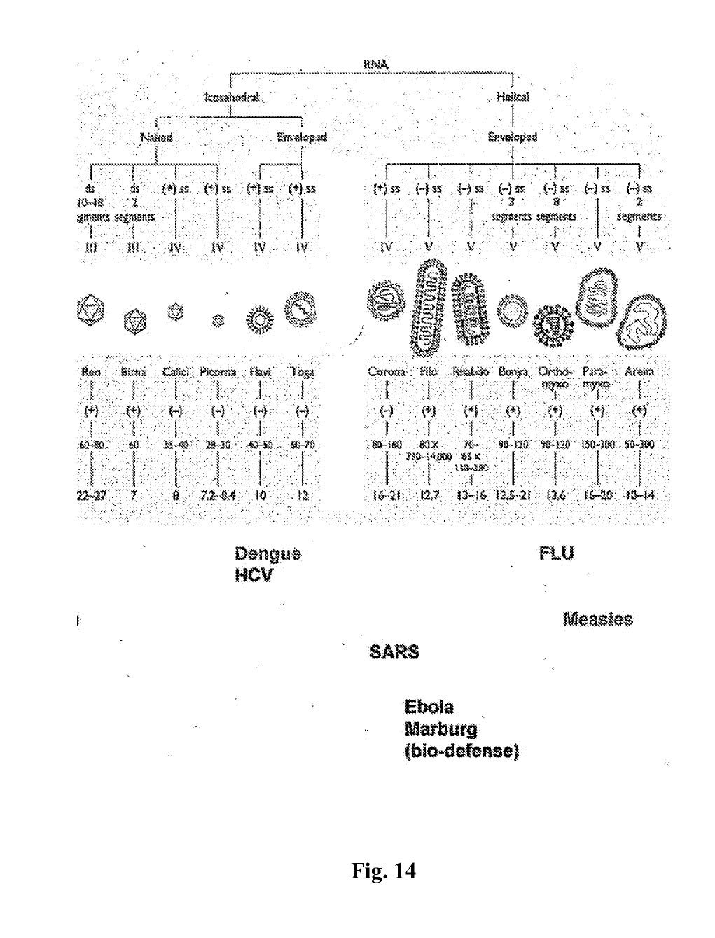

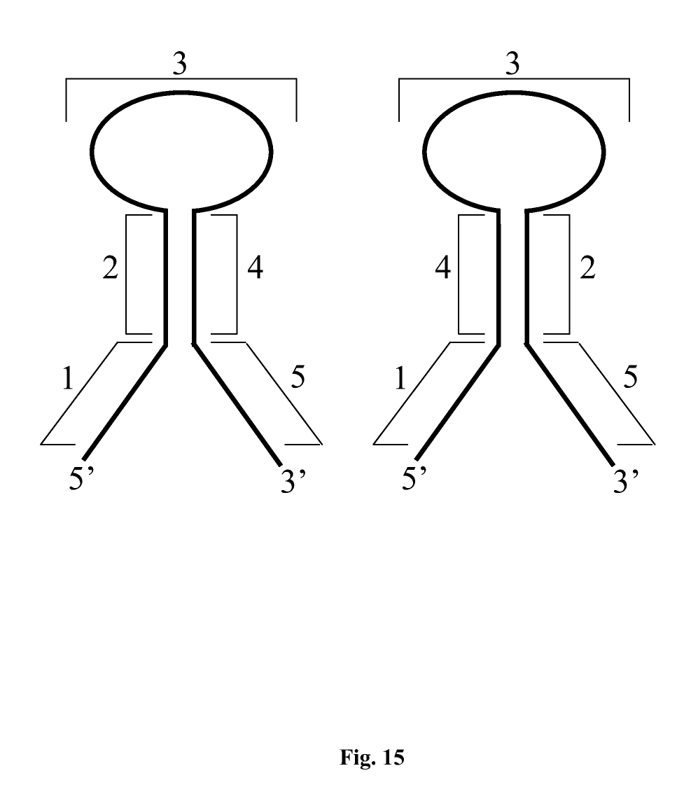

An illustrative embodiment of nucleic acids encoding a recombinant RNA virus is shown in FIG. 12. For example, plasmid-based rescue of Reovius can be used to introduce an additional non-coding dsRNA segments. For example, transfection of T7 polymerase-dependent plasmid encoding L1, L2, L3, M1, M2, M3, S1, S2, S3, and S4 flanked by a HDV ribozyme to generate specific 3 ends, yields replication competent virus in the presense of T7 polymerase (Kobayashi et al. 2007, Cell Host Microbe 1: 147-157). As viral egress specifically packages these 10 segments, introduction of a heterologous RNA requires the fusion of two segments in which the second segment is controlled by an internal ribosome entry site (IRES). The present of the IRES will permit the translation of the second 3′ product encoded on the same segment. This then allows original RNA segment to encode a heterologous RNA flanked by the necessary 5′ and 3′ sequences required for efficient packaging and reovirus polymerase recognition. In one embodiment, S3 and S4, encoding σ1 and σ2 respectively, being short RNA segments can be fused together as a single segment whereby σ2 is translated from an IRES (see FIG. 12). The original S4 RNA can therefore be used to deliver a heterologous RNA that will also package during virus replication.

In certain embodiments, a chimeric viral genome comprises: (a) a transcriptional start site for an double strand RNA dependent RNA polymerase; (b) a ribozyme cleavage site; (c) heterologous RNA; and (d) a ribozyme that cleaves the ribozyme cleavage site in section (b). In certain embodiments, one open reading frame of one viral genome segment is introduced into a second viral genomic segment so that one viral genomic segment contains two open reading frame. As a result, the total number of viral genomic segments including the chimeric viral genomic segment is the same as in the wild type virus. Also described herein are nucleic acids, such as DNA molecules, encoding such a chimeric togaviridae genome. In a specific embodiment, the chimeric viral genomic segment or the DNA molecule that encodes the chimeric viral genomic segment is isolated.

In certain embodiments, a chimeric viral genome comprises: (a) a transcriptional start site for an double strand RNA dependent RNA polymerase; (b) an open reading frame; (c) a ribozyme cleavage site; (d) heterologous RNA; and (e) a ribozyme that cleaves the ribozyme cleavage site in section (b). In certain embodiments, one open reading frame of one viral genome segment is introduced into a second viral genomic segment so that one viral genomic segment contains two open reading frame. Also described herein are nucleic acids, such as DNA molecules, encoding such a chimeric togaviridae genome. In a specific embodiment, the chimeric viral genomic segment or the DNA molecule that encodes the chimeric viral genomic segment is isolated.

5.4.2 Viruses

In certain embodiments, a recombinant RNA virus is derived from a reovirus, a rotavirus, orbivirus, or a Colorado tick fever virus. The virus can be any type, species, and/or strain of a reovirus, a rotavirus, orbivirus, or a Colorado tick fever virus.

5.4.3 Production of Recombinant RNA Viruses

Any method known to the skilled artisan can be used to rescue the virus that carries the heterologous RNA. In specific embodiments, the recombinant RNA viruses are isolated/purified.

In certain embodiments, the recombinant RNA virus is modified such that the virus is attenuated in the intended subject, e.g., a human patient. In specific embodiments, if the recombinant RNA virus is derived from a reovirus, the L1, L2, L3, M1, M2, M3, S1, S2, or S3 is mutated to achieve attenuation.

In certain embodiments, a recombinant RNA virus is used that does not normally infect the intended subject. Thus, in a specific embodiment, a recombinant RNA virus is derived from a non-human RNA virus wherein the intended subject is human.

In certain embodiments, the recombinant RNA virus is engineered such that the propagation of the virus can be terminated at will. For example, the recombinant RNA virus is a strain of the virus that is known to sensitive to an antiviral agent. If further propagation of the virus in th patient is no longer desired, the antiviral can be adminstered to discontinue propagation of the virus. In certain embodiments, the virus carries a suicide gene that prevents the virus from undergoing more than a complete life cycle thereby prevent further infection of the patient with the virus.

In certain embodiments, a recombinant RNA virus is engineered such that the virus can undergo only a limited number of replications in the subject. In more specific embodiments, the genome of a recombinant RNA virus is replicated 1, 2, 3, 4, 5, 6, 7, 8, 9, 10, 11, 12, 13, 14, 15, 16, 17, 18, 19, or 20 times in the subject. In other more specific embodiments, the recombinant RNA virus undergoes 1, 2, 3, 4, 5, 6, 7, 8, 9, 10, 11, 12, 13, 14, 15, 16, 17, 18, 19, or 20 times replication cycles in the subject.

In some embodiments, the attenuation can result, in part, from a mutation in a gene required for efficient replication of the recombinant RNA virus. Further, attenuation can result, in part, from a combination of one or more mutations in other viral genes.

In certain embodiments, the replication rate of a double-stranded RNA virus that carries a heterologous RNA is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of a double stranded RNA virus that carries a heterologous RNA is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of a double stranded RNA virus that carries a heterologous RNA is between 5% and 20%, between 10% and 40%, between 25% and 50%, between 40% and 75%, between 50% and 80%, or between 75% and 90% of the replication rate of the wild type virus from which the recombinant virus is derived under the same conditions.

In certain embodiments, attenuation of the virus can be mediated by segment truncation, or the generation of defective interfering (DI) particles in which the rescue is performed in the absent of one or essential non-structural genes. This will generate virus like particles that are unable to replicate unless the missing gene product is supplied in trans

5.4.4 Tropism

In certain embodiments, the natural tropism, or modified tropism, of the RNA virus from which the recombinant RNA virus is derived is used to target the effector RNA to a desired cell type, tissue, organ, or body part. Thus, if it is desired to affect expression of a target gene in, e.g., pulmonary tissue, a recombinant RNA virus that infects only pulmonary tissue is used.

In certain embodiments, the viral gene that is responsible for the tropism of the recombinant RNA virus is replaced with a gene from a second virus with a different tropism such that the recombinant RNA virus acquires the tropism of the second virus. In specific embodiments, the second virus is of the same type as the recombinant RNA virus. For example, a glycoprotein of the recombinant RNA virus can be replaced with the G gene of VSV, yielding a virus whose entry will not be restricted to any cell. In another specific embodiment, the coding regions of a glycoprotein of the recombinant RNA virus can be exchanged with gp41 and gp120, respectively, to obtain a recombinant RNA virus whose tropism would mimic that of HIV. In yet another embodiment, a glycoprotein of a recombinant RNA virus could be replaced with gpE1 of HCV to obtain a recombinant RNA virus that mimics the tropism of HCV.

In certain specific embodiments, the glycoprotein of the recombinant RNA virus is replaced with the glycoprotein of Borna Disease Virus to target neural tissue (Bajramovic 2003, J Virol 77:12222-12231).

In certain embodiments, the recombinant RNA virus expresses a soluble receptor (soR)-based expression construct fused to an epidermal growth factor (EGF) receptor targeting moiety to target the recombinant RNA virus to tumor cells (Verheije et al. 2009, J Virol 83:7507-7516).

5.5 Heterologous RNA

5.5.1 MicroRNA

In certain embodiments, the heterologous RNA encodes a primary transcript that comprises a microRNA precursor. microRNAs (miRNAs) are short non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nucleotide stem-loop precursor miRNA (precursor miRNA), which is exported from the nucleus to the cytomplasm by the protein exportin 5 (Exp5) where it is further cleaved by the cytoplasmic Dicer ribonuclease to generate the mature miRNA and antisense miRNA star (miRNA*) or passenger strand products. The mature miRNA is incorporated into a RNA-induced silencing complex (RISC), which recognizes target mRNAs through imperfect base pairing with the miRNA and most commonly results in translational inhibition or destabilization of the target mRNA.

A microRNA precursor can comprise the following elements in 5′ to 3′ direction:

5′ miRNA frame-passenger strand (sense strand or miRNA star)-central miRNA frame-mature miRNA (antisense- or guide strand)-3′ miRNA frame
or
5′ miRNA frame-mature miRNA (antisense- or guide strand)-central miRNA frame-passenger strand (sense strand or miRNA star)-3′ miRNA frame

In certain embodiments, the miRNA framework is modeled after the framework of a human precursor miRNA and is at least 90%, 91%, 92%, 93%, 94%, 95%, 96%, 97%, 98%, or at least 99%, or 100% identical to the miRNA framework of a human miRNA precursor. In certain, more specific embodiments, the miRNA framework is at least 90%, 91%, 92%, 93%, 94%, 95%, 96%, 97%, 98%, or at least 99%, or 100% identical to the precursor of human microRNA-30a (SEQ ID NO:1) (see, e.g., Zeng et al. 2002, Molecular Cell 9:1327-1333), or the precursor of human micro-RNA mir-585 (SEQ ID NO:2), or the precursor of human micro-RNA mir-55, or the precursor of human micro-RNA mir-142 (SEQ ID NO:4).

In certain embodiments, the miRNA framework is modeled after a canonical intronic miRNA (Kim et al. 2009, Nature Reviews Molecular Cell Biology 10: 126-139). In certain embodiments, the miRNA framework is modeled after a non-canonical intronic small RNA (mirtron) (Kim et al. 2009, Nature Reviews Molecular Cell Biology 10: 126-139).

In certain embodiments, the predicted structure of an artificial precursor miRNA is conserved relative to the human precursor miRNA after which the artificial precursor miRNA is modeled; the 5′ and 3′ sequences surrounding the artificial precursor miRNA are the same as the 5′ and 3′ flanking sequences of the primary transcript of the human miRNA after which the artifical miRNA is modeled; the bulge in the stem of the stem loop structure of the precursor miRNA is the same position and of the same length as in the human precursor miRNA after which the artificial miRNA is modeled.

In certain embodiments, the miRNA framework is an artificial framework. The artificial framework can be generated such that the miRNA precursor folds back on itself thereby forming a stem loop wherein the loop is located in the central miRNA frame. In certain embodiments, the loop is 10 nucleotides or longer and the stem is longer than the mature miRNA. In certain embodiments, precursor RNA has a 2 nucleotide 3′ overhang.

In certain embodiments, the 5′ miRNA frame is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long. In certain embodiments, the 5′ miRNA frame is between 1 to 10, 5 to 15, 10 to 20, 15 to 25, 20 to 30, 25 to 35, or 30 to 40 nucleotides long. In certain embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the 5′ miRNA frame are complementary to nucleotides in the 3′ miRNA frame in the stem loop structure. In certain embodiments, between 1 to 10, 5 to 15, 10 to 20, 15 to 25, 20 to 30, 25 to 35, or 30 to 40 nucleotides in the 5′ miRNA frame are complementary to nucleotides in the 3′ miRNA frame in the stem loop structure. In certain embodiments, the complementary nucleotides are in 1, 2, 3, 4, or 5 clusters of 1, 2, 3, 4, 5, 6, 7, 8, 9, 10, 11, 12, 13, 14, 15, 16, 17, 18, 19, or 20 consecutive nucleotides.

In certain embodiments, the 3′ miRNA frame is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long. In certain embodiments, the 3′ miRNA frame is between 1 to 10, 5 to 15, 10 to 20, 15 to 25, 20 to 30, 25 to 35, or 30 to 40 nucleotides long. In certain embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the 3′ miRNA frame are complementary to nucleotides in the 5′ miRNA frame in the stem loop structure. In certain embodiments, between 1 to 10, 5 to 15, 10 to 20, 15 to 25, 20 to 30, 25 to 35, or 30 to 40 nucleotides in the 3′ miRNA frame are complementary to nucleotides in the 5′ miRNA frame in the stem loop structure. In certain embodiments, the complementary nucleotides are in 1, 2, 3, 4, or 5 clusters of 1, 2, 3, 4, 5, 6, 7, 8, 9, 10, 11, 12, 13, 14, 15, 16, 17, 18, 19, or 20 consecutive nucleotides.

In certain embodiments, the 5′ miRNA frame is at least 10%, 20%, 30%, 40%, 50%, 60%, 70%, 80%, or at least 90% complementary to the 3′ miRNA frame. In certain embodiments, the 5′ miRNA frame is between 10% to 20%, 20% to 30%, 30% to 40%, 40% to 50%, 50% to 60%, 60% to 70%, 70% to 80%, or 80% to 90% complementary to the 3′ miRNA frame.

In certain embodiments, the central miRNA frame is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long. In certain embodiments, the central miRNA frame is between 1 to 10, 5 to 15, 10 to 20, 15 to 25, 20 to 30, 25 to 35, or 30 to 40 nucleotides long. In certain embodiments, 1, 2, 3, 4, 5, 6, 7, 8, 9, 10, 11, 12, 13, 14, 15, 16, 17, 18, 19, or 20, nucleotides are complementary to nucleotides in the central miRNA frame in the loop structure. In certain embodiments, between 1 to 10, 5 to 15, to 20, 15 to 25, 20 to 30, 25 to 35, or 30 to 40 nucleotides are complementary to nucleotides in the central miRNA frame in the loop structure. In certain embodiments, the complementary nucleotides are in 1, 2, 3, 4, or 5 clusters of 1, 2, 3, 4, 5, 6, 7, 8, 9, 10, 11, 12, 13, 14, 15, 16, 17, 18, 19, or 20 consecutive nucleotides.

In certain embodiments, the mature miRNA is a human miRNA. In certain embodiments, the mature miRNA is an artificial mature miRNA. The mature miRNA can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long. In certain more specific embodiments, the mature miRNA is 20, 21, 22, 23, or 24 nucleotides long. The selection of the sequence of a mature miRNA is described in the section “Sequence of Mature miRNA.”

In certain embodiments, the miRNA precursor is a human miRNA precursor.

In certain embodiments, the passenger strand is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides long. In certain embodiments, the passenger strand is at least 90%, 91%, 92%, 93%, 94%, 95%, 96%, 97%, 98%, or at least 99%, or 100% complementary to the mature miRNA. In certain embodiments, the complementary nucleotides are in 1, 2, 3, 4, or 5 clusters of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 consecutive nucleotides. In certain embodiments, the passenger strand is 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, or 120% of the length of the mature miRNA.

In certain embodiments, the hybrid between passenger strand and mature miRNA comprises 1, 2, 3, 4, or 5 bulges, i.e., regions of non-complementary nucleotides. A bulge can be 1, 2, 3, 4, 5, 6, 7, 8, 9, or 10 nucleotides long.

An artificial precursor can be tested in an in vitro assay for its ability to serve as a substrate for the Dicer endoribonuclease (see Section 5.9.3.3). In certain embodiments, the Dicer endoribonuclease processes the artificial precursor at least 10%, 20%, 30%, 40%, 50%, 60%, 70%, 80%, or at least 90%, 95%, 98%, 99% or 100% as efficiently as a wild type substrate, i.e., the wild type miRNA precursor after which the artificial miRNA precursor is modeled. Without being bound by theory, the product of the Dicer enzymatic reaction is a 21-24 nucleotide double-stranded RNA with two base 3′ overhangs and a 5′ phosphate and 3′ hydroxyl group. Further, without being limited by theory, the product of the Dicer enzymatic reaction is incorporated into the RNA-induced silencing complex (RISC) and the passenger strand is cleaved and removed by Argonaute 2 (AGO2).

In certain embodiments, the primary transcript is the precursor miRNA. Precise 3′ and 5′ ends can be generated by using, e.g., appropriate splicing sites or by incorporating RNAzymes.

In certain embodiments, the primary transcript comprises the precursor miRNA surrounded by extra RNA sequences. In certain embodiments, the extra RNA sequences are transcribed from flanking sequences of the template heterolous RNA. In certain embodiments, the extra RNA sequences are added after transcription. In certain embodiments, the primary transcript is capped and polyadenylated. Without being bound by theory, the primary transcript is processed by the Drosha ribonuclease III enzyme to produce an miRNA precursor. In certain embodiments, the loop of the primary transcript is 10 nucleotides or longer; and/or the stem of the primary transcript is longer than the mature miRNA; and/or the stem of the primary transcript is longer than the stem of the precursor miRNA; and/or the primary transcript has at least 40 nucleotides of additional sequences on each side of the precursor miRNA; and/or at least 10%, 20%, 30%, 40%, 50%, 60%, 70%, 80%, or at least 90% or 100% of the extra RNA sequences that flank the precursor RNA are single stranded RNA. In a more specific embodiment, at least 3 nucleotides of the extra RNA sequence are single stranded. In certain embodiments, the primary transcript is between 50 and 100, 75 and 150, 100 and 200, 150 and 250, 200 and 300, 250 and 350, 300 and 500, 400 and 600, 500 and 700, 600 and 800, 700 and 900, 800 and 1,000 nucleotides long.

An artificial precursor can be tested in an in vitro assay for its ability to serve as a substrate for the Drosha ribonuclease (see Section 5.9.3.1). In certain embodiments, the Drosha ribonuclease processes the artificial precursor at least 10%, 20%, 30%, 40%, 50%, 60%, 70%, 80%, or at least 90% or 100% as efficiently as a wild type substrate. In certain embodiments, the artificial precursor serves as substrate of the microprocessor complex, consisting of the proteins Drosha and GiGeorge syndrom critical region gene 8 (DGCR8) (see Section 5.9.3.2). In certain embodiments, the microprocessor complex processes the artificial precursor at least 10%, 20%, 30%, 40%, 50%, 60%, 70%, 80%, or at least 90% or 100% as efficiently as a wild type substrate.

In certain embodiments, the primary transcript is between 0.5 and 1.5, 1 and 2, 1.5 and 2.5, 2 and 3, 2.5 and 3.5, 3 and 4, 3.5 and 4.5, 4 and 5, 4.5 and 5.5, 5 and 6, 5.5 and 6.5, 6 and 7, 6.5 and 7.5, 7 and 8, 7.5 and 8.5, 8 and 9, 8.5 and 9.5, 9 and 10, 9.5 and 10.5, 10 and 15, 12.5 and 17.5, 15 and 20, 17.5 and 22.5, 20 and 25, 22.5 and 27.5, and 30 kilo-nucleotides long. In certain embodiments, the primary transcript contains tandem repeats of the mature miRNA and the and the passenger strand as follows (numbers after the passenger strand and mature miRNA indicate the number of the repeat, n can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or higher):

5′ miRNA frame-passenger strand-1- . . . -passenger strand-n-central miRNA frame-mature miRNA-n- . . . -mature miRNA-1-3′ miRNA frame
or
5′ miRNA frame-mature miRNA-1- . . . -mature miRNA-n-central miRNA frame-passenger strand-n- . . . -passenger strand-1-3′ miRNA frame

In certain embodiments, the primary transcript from the heterologous RNA is tested in an in vitro assay for cleavage by the Drosha ribonuclease III enzyme (see, e.g., Zeng and Cullen, 2005, J Biol Chem 280:27595-27603 and Section 5.9.3.1).

In certain embodiments, the heterologous RNA is flanked by a splice donor and a splice acceptor site. Upon transcription a lariat that encompasses th heterologous RNA is formed. Without being bound by theory, the lariat is debranched and folds to form the precursor miRNA. In certain other embodiments, the heterologous RNA is flanked by a ribozyme and its ribozyme cleavage sites. Upon transcription, the ribozyme cleaves the heterologous RNA from the transcript. In certain other embodiments, the heterologous RNA is flanked by two ribozymes and their ribozyme cleavage sites. Upon transcription, the ribozymes cleave the heterologous RNA from the transcript.

In certain embodiments, once the transcribed heterologous RNA is processed by Drosha, the Drosha product has a double stranded stem that is longer than 14 nucleotides and has a 1 to 8 3′ overhang. Without being bound by theory, such a Drosha product can be transported from the nucleus into the cytoplams by Exportin 5.

Without being bound by theory, following Dicer cleavage, the relative thermodynamic stability of the passenger strand versus the mature miRNA determines which strand is incorporated into RISC. Further, without being limited by theory, the relative thermodynamic instability at the 5′ end of a strand of a Dicer product favors its loading into RISC. Schwarz et al. 2003, Cell 115:199-208.

In certain embodiments, the thermodynamic stability of the 5′ end of one strand is decreased over the other to favor the loading of that strand into RISC. Thermodynamic stability can be changed by changing one of the stem's ends. For example, GC pairing is increased at the 3′ end of the strand that is intended to become the miRNA (i.e., GC pairing is increased at the 5′ end of the strand that is intended to become the passenger strand). Without being bound by theory, the strand with less GC pairing at its 5′ end is more likely to be loaded into RISC. Further without being bound by theory, as the stem is determined by the 5′ end, changing one or two bases at the 3′ end of your artificial microRNA is unlikely to adversely affect targeting to RISC. In certain embodiments, the thermodynamic stability is reversed such that the passenger strand is incorporated into RISC.

In certain embodiments, if the recombinant RNA virus is derived from a virus that replicates in the cytoplasm, the heterologous RNA, upon transcription, is a substrated for Dicer. In certain embodiments, if the recombinant RNA virus is derived from a virus that replicates in the nucleus, the heterologous RNA, upon transcription, is a substrated for Drosha.

5.5.1.1 Sequence of mature miRNA

In certain embodiments, the mature miRNA is a human miRNA. In other embodiments, the mature miRNA is an artificial miRNA (amiRNA) whose sequence is derived from the sequence of the desired target. The desired target can be a gene of the genome of a patient, of a pathogen, and/or a gene of the recombinant RNA virus itself (see Section 5.7). In certain embodiments, the mature miRNA is an amiRNA that has multiple targets, e.g., the miRNA can target multiple variations of a certain gene or multiple variations of a particular pathogen, e.g., different isolates/strains of a particular virus (see, e.g., Israsena et al., 2009, Antiviral Research 84:76-83). Software tools for predicting miRNA targets can be used to verify that the artificial miRNA targets the desired target (Bartel 2009, Cell 136:215-233).

In certain embodiments, the mature miRNA is 15, 16, 17, 18, 19, 20, 21, 22, 23, 24, 25, 26, 27, 28, 29, or 30 nucleotides long. In certain embodiments, the mature miRNA is between 10 to 20, 15 to 25, 20 to 30, or 25 to 35 nucleotides long. In more specific embodiments, the mature miRNA is 20, 21, 22, 23, or 24 nucleotides long.

In certain embodiments, at least 50%, 55%, 60%, 65%, 70%, 75%, 80%, 85%, 86%, 87%, 88%, 89%, 90%, 91%, 92%, 93%, 94%, 95%, 96%, 97%, 98%, or at least 99%, or 100% of the nucleotides of the mature miRNA are complementary to the target sequence thus allowing for Watson-Crick pairing in between the complementary nucleotides. In certain embodiments, nucleotides 2-7 at the 5′ end of the mature miRNA (the “seed”) are complementary to the target thus allowing for perfect base pairing between mature miRNA and its target between nucleotides 2-7 of the mature miRNA.

In certain embodiments, the nucleotide sequence of the mature miRNA is compared to the entire transcriptome to avoid off-target effects. In a specific embodiment, the complementary sequence to the mature miRNA is a unique sequence in the human transcriptome. In specific embodiments, the complementary sequence is unique to a gene of a pathogen and cannot be found in the transcriptome of the subject.

In certain embodiments, the target sequence of the miRNA is located in the 3′ untranslated region (UTR) of the target gene.

5.5.2 siRNA

In certain embodiments, the heterologous RNA gives rise to siRNA. In certain embodiments, siRNAs are between 19 to 25 nucleotide long double stranded RNAs. Transcription from the heterologous RNA results in double stranded RNA molecule that comprises a portion that is complementary to the target gene of interest. Without being bound by theory, the double stranded RNA is processed by the Dicer complex to siRNA.

Without being bound by theory, the efficacy of siRNAs for individual targets normally depends on different factors, such as thermodynamic stability, structural features, target mRNA accessibility, and additional position-specific determinants. See, Lopez-Fraga et al. 2009, Biodrugs 23:305-332. In certain embodiments, siRNAs to be used with the present methods and compositions should be between 19 and 25 nucleotides long, should have 30 symmetric dinucleotide overhangs, low guanine-cytosine content (between 30% and 52%) and specific nucleotides at certain positions. For example, features that increase siRNA efficacy are the presence of an adenine or uracil in position 1, adenosine in position 3, a uracil in positions 7 and 11, a guanine in position 13, a uracil or adenine in position 10 (this is the site for RISC mediated cleavage), a guanine in position 21 and/or the absence of guanines or cytosine at position 19 of the sense strand (see Dykxhoorn and Lieberman 2006, Annu Rev Biomed Eng 8:377-402).

In general, enrichment in adenosines and uracils along the first 6-7 base pairs of the sequence, and consequently, weak hydrogen bonding, allows the RISC to easily unravel the doublestranded duplex and load the guide strand. siRNA duplexes should also be thermodynamically flexible at their 30 end, i.e. at positions 15-19 of the sense strand. This correlates with their silencing efficacy, such that the presence of at least one adenosine-uracil pair in this region would decrease the internal stability and increase the silencing efficacy. In contrast, internal repeats or palindrome sequences decrease the silencing potential of the siRNAs.

Another consideration that needs to be taken into account when designing a siRNA sequence is the nature of the target sequence. Under certain circumstances it will be preferable to include all the splice variants and isoforms for the design of the siRNA, whereas in other instances they should be specifically left out. Similarly, attention should be paid to choice of sequences within the coding region of the target gene sequence, as gene silencing is an exclusively cytoplasmic process.

Computer-based algorithms can help in the design of optimal siRNA sequences for any given gene, and will consider properties such as thermodynamic values, sequence asymmetry, and polymorphisms that contribute to RNA duplex stability.

Without being bound by theory, the generation of siRNA is only dependent on Dicer activity, but not on Drosha. Accordingly, the primary transcript of the heterologous RNA is a substrate for Dicer (see Section 5.9.3.3).

In specific embodiments, cytoplasmic viruses are used to generate a recombinant RNA virus for the delivery of siRNA.

In certain embodiments, the heterologous RNA encodes a long hairpin structure. In certain other embodiments, two separate heterologous RNAs are introduced into the recombinant RNA virus which together provide the sense-antisense pairs to form a dsRNA.

In certain embodiments, the recombinant RNA virus is a double strand RNA virus and the heterologous RNA is flanked by promoters that transcribe the RNA in opposite directions to generate convergent transcripts.

5.5.3 shRNA

In certain embodiments, the heterologous RNA encodes a short hairpin RNA (shRNA). In certain embodiments, the primary transcript from the heterologous RNA folds into a hairpin loop with the following properties: 3′ UU-overhangs, stem lengths is between 25 to 29 nucleoties and loop size is between 4 to 23 nucleotides. In certain embodiments, complementarity between the portion of the stem that binds to the target mRNA (antisense strand) and the mRNA is 100%.

Without being bound by theory, the primary transcript from the heterologous RNA is a substrate of Exportin 5 (see Section 5.9.3.4). In certain embodiments, if the recombinant RNA virus is a cytomplasmic virus, the primary transcript from the heterologous RNA is a substrate of Dicer (see Section 5.9.3.3).

5.5.4 RNA Sponge

In certain embodiments, a heterologous RNA is tandem repeats of complementary RNA to a desired target gene. In certain embodiments, the heterologous RNA contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 repeats. In certain embodiments, the heterologous RNA contains between 1 to 10, 5 to 15, 10 to 20, 15 to 25, 20 to 30, 25 to 35, or 30 to 40 repeats. In certain embodiments, each repeat is between 10 to 20, 15 to 25, 20 to 30, 25 to 35, 30 to 40, 35 to 45, or 40 to 50 nucleotides long. In certain embodiments, the segments between the repeats are 0 to 10, 5 to 15, 10 to 20, 15 to 25, 20 to 30, 25 to 35, 30 to 40, 35 to 45, or 40 to 50 nucleotides long.

5.5.5 Antisense RNA

In certain embodiments, heterologous RNA is transcribed to give rise to an antisense RNA that is complementary to an mRNA of a target gene.

5.5.6 svRNA

In certain embodiments, heterologous RNA is transcribed to give rise to an svRNA. In specific embodiments, the heterologous RNA is transcribed to mimic an svRNA, such as an svRNA of influenza virus (see, e.g., Perez et al., “Influenza A virus-generated small RNAs regulate the switch from transcription to replication,” PNAS, published online on Jun. 1, 2010).

In certain embodiments, a small viral RNAs (svRNAs) is an svRNA of an orthomyxovirus, e.g., influenza virus. Without being bound by theory, svRNAs expressed by influenza viruses are involved in regulating viral replication by, e.g., regulating the switch from transcription to replication of the viral genome. Without being bound by any theory, compounds that modulate the expression or activity of such small viral RNAs can modulate the switch between transcription and replication of the viral genome and, thus, can modulate the production of viral particles. In one aspect, compounds that modulate the switch between transcription and replication of the Orthomyxovirus viral genome may be used. In other aspects, compounds that modulate the switch between transcription and replication of the Orthomyxovirus viral genome can be used with a recombinant RNA virus that is derived from an orthomyxovirus. In certain aspects, compounds that modulate the switch between transcription and replication of the Orthomyxovirus viral genome can be used to selectively modulate the production of one or more Orthomyxovirus genome segments or mRNA transcripts and, in turn, can selectively modulate the production of one or more Orthomyxovirus proteins or a heterologous RNA.

In specific embodiments, an svRNA is a single stranded RNA identical to the 5′ end of the viral genomic RNA (vRNA) and complementary to the 3′ end of the complementary viral RNA genome (cRNA). In one embodiment, an svRNA is generated from the 5′ end(s) of Orthomyxovirus genomic RNA (alternatively referred to herein as “vRNA”) by RNA-dependent RNA polymerase (RdRp) cleavage. In one embodiment, an svRNA is generated from the 3′ end(s) of the Orthomyxovirus genomic cRNA by RdRp machinery. In one embodiment, the svRNA interacts with the 3′ end of the vRNA. In another embodiment, the svRNA interacts with the 3′ end of the cRNA. In some embodiments, the svRNA interacts with the 3′ ends of both Orthomyxovirus vRNA and cRNA. svRNAs are described in U.S. Provisional Patent Application No. 61/327,384 filed on Apr. 23, 2010.

5.6 Compositions

The recombinant RNA viruses provided herein may be incorporated into compositions. In a specific embodiment, the compositions are pharmaceutical compositions, including immunogenic compositions (e.g., vaccine formulations). The pharmaceutical compositions provided herein can be in any form that allows for the composition to be administered to a subject. In a specific embodiment, the pharmaceutical compositions are suitable for veterinary and/or human administration. The compositions may be used in methods of preventing or treating a disease.

In one embodiment, a pharmaceutical composition comprises a recombinant RNA virus, in an admixture with a pharmaceutically acceptable carrier. In some embodiments, a pharmaceutical composition may comprise one or more other therapies in addition to a recombinant RNA virus.

As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E.W. Martin. The formulation should suit the mode of administration.

In a specific embodiment, pharmaceutical compositions are formulated to be suitable for the intended route of administration to a subject. For example, the pharmaceutical composition may be formulated to be suitable for parenteral, oral, intradermal, transdermal, colorectal, intraperitoneal, or rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration.

In certain embodiments, biodegradable polymers, such as ethylene vinyl acetate, polyanhydrides, polyethylene glycol (PEGylation), polymethyl methacrylate polymers, polylactides, poly(lactide-co-glycolides), polyglycolic acid, collagen, polyorthoesters, and polylactic acid, may be used as carriers. In some embodiments, the recombinant RNA viruses are prepared with carriers that increase the protection of the recombinant RNA virus against rapid elimination from the body, such as a controlled release formulation, including implants and microencapsulated delivery systems. Methods for preparation of such formulations will be apparent to those skilled in the art. Liposomes or micelles can also be used as pharmaceutically acceptable carriers. These can be prepared according to methods known to those skilled in the art, for example, as described in U.S. Pat. No. 4,522,811. In certain embodiments, the pharmaceutical compositions comprise one or more adjuvants.

In specific embodiments, pharmaceutical compositions described herein are monovalent formulations. In other embodiments, pharmaceutical compositions described herein are multivalent formulations. In one example, a multivalent formulation comprises one or more recombinant RNA viruses.

In certain embodiments, the pharmaceutical compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative. In a specific embodiment, thimerosal is used during the manufacture of a pharmaceutical composition described herein and the thimerosal is removed via purification steps following production of the pharmaceutical composition, i.e., the pharmaceutical composition contains trace amounts of thimerosal (<0.3 μg of mercury per dose after purification; such pharmaceutical compositions are considered thimerosal-free products).

In certain embodiments, the pharmaceutical compositions described herein additionally comprise egg protein (e.g., ovalbumin or other egg proteins). The amount of egg protein in the pharmaceutical compositions described herein may range from about 0.0005 to about 1.2. μg of egg protein to 1 ml of pharmaceutical composition. In other embodiments, the pharmaceutical compositions described herein do not comprise egg protein.

In certain embodiments, the pharmaceutical compositions described herein additionally comprise one or more antimicrobial agents (e.g., antibiotics) including, but not limited to gentamicin, neomycin, polymyxin (e.g., polymyxin B), and kanamycin, streptomycin. In other embodiments, the pharmaceutical compositions described herein do not comprise any antibiotics.

In certain embodiments, the pharmaceutical compositions described herein additionally comprise gelatin. In other embodiments, the pharmaceutical compositions described herein do not comprise gelatin.

In certain embodiments, the pharmaceutical compositions described herein additionally comprise one or more buffers, e.g., phosphate buffer and sucrose phosphate glutamate buffer. In other embodiments, the pharmaceutical compositions described herein do not comprise buffers.

In certain embodiments, the pharmaceutical compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the pharmaceutical compositions described herein do not comprise salts.

In specific embodiments, the pharmaceutical compositions described herein do not comprise one or more additives commonly found in vaccine formulations, e.g., influenza virus vaccine formulations. Such vaccines have been described (see, e.g., International Aplication No. PCT/IB2008/002238 published as International Publication No. WO 09/001,217 which is herein incorporated by reference in its entirety).

The pharmaceutical compositions described herein can be included in a container, pack, or dispenser together with instructions for administration.

The pharmaceutical compositions described herein can be stored before use, e.g., the pharmaceutical compositions can be stored frozen (e.g., at about −20° C. or at about −70° C.); stored in refrigerated conditions (e.g., at about 4° C.); or stored at room temperature (see International Aplication No. PCT/IB2007/001149 published as International Publication No. WO 07/110,776, which is herein incorporated by reference in its entirety, for methods of storing compositions comprising influenza vaccines without refrigeration).

In a specific embodiment, provided herein are compositions comprising live, recombinant RNA virus, wherein the virus is influenza virus. In some embodiments, the composition comprising live, recombinant RNA virus, wherein the virus is influenza virus, is an immunogenic composition (e.g., a vaccine). In another specific embodiment, provided herein are compositions comprising live, recombinant RNA virus, wherein the virus is sindbis virus.

In certain embodiments, compositions comprising live, recombinant RNA virus comprise virus with an altered tropism, i.e., the tropism of the recombinant RNA virus differs from the natural tropism of the virus (e.g., the tropism of the wild-type virus.

In certain embodiments, compositions comprising live, recombinant RNA virus comprise virus that is attenuated in subjects to which the compositions are administered. Such attenuated recombinant RNA viruses can be naturally attenuated (i.e., the virus naturally does not cause disease in a subject) or can be engineered to be attenuated (i.e., the virus is genetically altered so that it does not cause disease in a subject).

5.6.1 Adjuvants

In certain embodiments, the compositions described herein (particularly the immunogenic compositions) comprise, or are administered in combination with, an adjuvant. The adjuvant for administration in combination with a composition described herein may be administered before, concommitantly with, or after administration of said composition. In some embodiments, the term “adjuvant” refers to a compound that when administered in conjunction with or as part of a composition described herein augments, enhances and/or boosts the immune response to a composition described herein. Adjuvants can enhance an immune response by several mechanisms including, e.g., lymphocyte recruitment, stimulation of B and/or T cells, and stimulation of macrophages.

Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine, or other immunopotentiating agents.

5.7 Uses of Recombinant RNA Viruses

5.7.1 Modulation of Gene Expression and miRNA Targeting

In one aspect, the recombinant RNA viruses described herein can be used to modulate gene expression. Without being limited by theory, the recombinant RNA viruses described herein can be engineered to produce effector RNA specific to a target gene, such that when the effector RNA comes in contact with the mRNA transcribed from the target gene, expression of the target gene is modulated. The target gene modulated by an effector RNA expressed by a recombinant RNA virus described herein can be, without limitation, a gene of a subject, a gene of a plant, a gene of a pathogen, or a gene of a cell or cell line. Thus, described herein is a method for reducing the expression of a target gene in a subject by administering a recombinant RNA virus that delivers an effector RNA to the subject. Further provided herein is a method for reducing the titers of a pathogen in a subject by administering a recombinant RNA virus that delivers an effector RNA that targets the pathogen to the subject.

The ability of an effector RNA to modulate the expression of a target gene can be assessed using approaches known to those skilled in the art as well as the approaches described herein, such as techniques for measuring RNA expression (e.g., mRNA expression) or protein expression (see Section 5.9.4, infra) Those skilled in the art will recognize that if the level of RNA expression (e.g., mRNA expression) and/or the level or protein expression from a target gene in the presence of an effector RNA is reduced relative to the level of RNA expression and/or the level or protein expression in the absence of the effector RNA, then the target gene has been modulated by the effector RNA. Conversely, if the level of RNA expression (e.g., mRNA expression) and/or the level or protein expression from a target gene in the presence of an effector RNA is the same as the level of RNA expression and/or the level or protein expression in the absence of the effector RNA, then the target gene has not been modulated by the effector RNA.

In some embodiments, a target gene may be modulated by an effector RNA such that the RNA expression (e.g., mRNA expression) by the target gene is completely reduced, i.e., no RNA is produced by the target gene. In other embodiments, a target gene may be modulated by an effector RNA such that the RNA expression (e.g., mRNA expression) by the target gene is not completely reduced, but is reduced relative to the level of RNA expression by the target gene under normal conditions (i.e., in the absence of the effector RNA), e.g., the expression may be reduced by 5, 10, 15, 20, 25, 30, 35, 40, 45, 50, 55, 60, 65, 70, 75, 80, 85, 90, 95, 96, 97, 98, or 99% or by 2-, 3-, 4-, 5-, 6-, 7-, 8-, 9-, 10-, 15-20-, 25-, 50-, or 100-fold, or greater than 100-fold.

In some embodiments, a target gene may be modulated by an effector RNA such that the protein expression by the target gene is completely reduced, i.e., no protein is produced by the target gene. In other embodiments, a target gene may be modulated by an effector RNA such that the protein expression by the target gene is not completely reduced, but is reduced relative to the level of protein expression by the target gene under normal conditions (i.e., in the absence of the effector RNA), e.g., the expression may be reduced by 5, 10, 15, 20, 25, 30, 35, 40, 45, 50, 55, 60, 65, 70, 75, 80, 85, 90, 95, 96, 97, 98, or 99% or by 2-, 3-, 4-, 5-, 6-, 7-, 8-, 9-, 10-, 15-20-, 25-, 50-, or 100-fold, or greater than 100-fold.

In another aspect, the recombinant RNA viruses described herein can be used to target other miRNAs. Without being limited by theory, the recombinant RNA viruses described herein can be engineered to produce effector RNA specific to a target miRNA. The target miRNA modulated by an effector RNA expressed by a recombinant RNA virus described herein can be, without limitation, an miRNA of a subject, an miRNA of a plant, an miRNA of a pathogen, or an miRNA of a cell or cell line. Thus, described herein is a method for modulating (e.g., reducing) the expression of a target miRNA in a subject by administering a recombinant RNA virus that delivers an effector RNA to the subject. Further provided herein is a method for reducing the titers of a pathogen in a subject by administering a recombinant RNA virus that delivers an effector RNA that targets the an miRNA of pathogen to the subject.

5.7.2 Prevention or Treatment of Disease

In one aspect, the recombinant RNA viruses described herein can be used to prevent or treat disease in a subject. Without being limited by theory, the recombinant RNA viruses described herein can be engineered to produce effector RNA that target genes of a subject that are implicated in disease due to the fact that the genes are overexpressed or ectopically expressed. Alternatively, the recombinant RNA viruses described herein can be engineered to produce effector RNA molecules that target genes of a pathogen (e.g., a virus or bacteria), i.e., the effector RNA targets a gene of the pathogen that is essential for propagation or survival of the pathogen. Further, the recombinant RNA viruses described herein can be engineered to produce effector RNA that targets miRNA of a subject or miRNA of a pathogen that is implicated in disease. As such, any disease for which benefit may be obtained by administering a recombinant RNA virus is encompassed in the methods of preventing or treating disease described herein. In certain embodiments, pathogens include, but are not limited to, bacteria, viruses, yeast, fungi, archae, prokaryotes, protozoa, parasites, and algae.

In certain embodiments, the disease treated in accordance with the methods described herein is a respiratory disease. Respiratory diseases include, without limitation, diseases of the lung, pleural cavity, bronchial tubes, trachea, upper respiratory tract and of the nerves and muscles of breathing. Exemplary respiratory diseases that can be treated in accordance with the methods described herein include viral infections, bacterial infections, asthma, cancer, chronic obstructive pulmonary disorder (COPD), emphysema, pneumonia, rhinitis, tuberculosis, bronchitis, laryngitis, tonsilitis, and cystic fibrosis.

In certain embodiments, the disease treated in accordance with the methods described herein is cancer. Non-limiting examples of cancer that can be treated in accordance with the methods described herein include: leukemia, lymphoma, myeloma, bone and connective tissue sarcomas, brain cancer, breast cancer, ovarian cancer, kidney cancer, pancreatic cancer, esophageal cancer, stomach cancer, lung cancer (e.g, small cell lung cancer (SCLC), non-small cell lung cancer (NSCLC), throat cancer, and mesothelioma), and prostate cancer.

In certain embodiments, the disease treated in accordance with the methods described herein is a disease associated with the need to regulate the levels of cholesterol in an individual, e.g., the disease is one associated with high cholesterol. Non-limiting examples of diseases associated with high cholesterol include heart disease, stroke, peripheral vascular disease, diabetes, and high blood pressure.

In certain embodiments, the disease treated in accordance with the methods described herein is a disease caused by viral infection. A non-limiting list of disease-causing viruses includes: respiratory syncytial virus (RSV), influenza virus (influenza A virus, influenza B virus, or influenza C virus), human metapneumovirus (HMPV), rhinovirus, parainfluenza virus, SARS Coronavirus, human immunodeficiency virus (HIV), hepatitis virus (A, B, C), ebola virus, herpes virus, rubella, variola major, and variola minor.

In certain embodiments, the disease treated in accordance with the methods described herein is a disease caused by bacterial infection. A non-limiting list of disease-causing bacteria includes: Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and Treponema pallidum.

In a specific embodiment, the effector RNA produced by a recombinant RNA virus described herein targets a gene of a pathogen that infects subjects, wherein the effector RNA targets a gene of the pathogen that is essential for propagation or survival of the pathogen. In another specific embodiment, the effector RNA produced by a recombinant RNA virus described herein targets a gene of a pathogen that infects plants, wherein the effector RNA targets a gene of the pathogen that is essential for propagation or survival of the pathogen.

In certain embodiments, the disease treated in accordance with the methods described herein is an autoimmune disease. Examples of autoimmune diseases that can be treated by the methods described herein include, but are not limited to, Addison's disease, Behcet's disease, chronic active hepatitis, chronic fatigue immune dysfunction syndrome (CFIDS), chronic inflammatory demyelinating polyneuropathy, Churg-Strauss syndrome, Crohn's disease, Graves' disease, Guillain-Barre, Myasthenia Gravis, Reiter's syndrome, rheumatoid arthritis, sarcoidosis, Sjögren's syndrome, and systemic lupus erythematosus.

Other diseases that can be treated in accordance with the methods described herein include, without limitation, Alzheimer's disease, Parkinson's disease, cardiovascular disease, allergic diseases, diabetes, Huntington's disease, Fragile X Syndrome, glaucoma, and psoriasis.

Many genes have been implicated in disease in both human and non-human subjects. For example, the E4 allele of the apolipoprotein E (ApoE) gene (GENE ID NO: 348) has been linked to Alzheimer's disease (see, e.g., Kim et al., 2009, Neuron 63(3):287-303). Genes have also been implicated in cancer: the epidermal growth factor receptor (EGFR) gene and the KRAS gene (GENE ID NO: 3845) have been implicated in multiple cancer types (see, e.g., Lynch et al., 2004, N. Engl. J. Med. 350(21):2129-39; and Kranenburg, 2005, Biochim. Biophys. Acta 1756(2):81-2), and the pituitary transforming gene (PTTG; GENE ID NO: 9232) has been found to be overexpressed in lung cancer. Indeed, siRNA that targets PTTG has been shown to be an effective mechanism for preventing tumor growth in mice transfected with lung cancer tumor cells (see Kakar and Malik, 2006, Int. J. Oncology 29(2):387-395). Another human gene, ELANE (GENE ID NO: 1991) has been implicated in emphysema and chronic obstructive pulmonary disorder. Thus, in accordance with the methods for preventing or treating disease described herein, the foregoing genes, as well as any other genes implicated in disease, could be targeted with effector RNA produced by a recombinant RNA virus described herein.

Certain genes of pathogens such as viruses and bacteria are essential to the replication and/or survival of the pathogen in subjects and thus required for virulence of the pathogen. In some embodiments, the effector RNA produced by a recombinant RNA virus described herein targets a gene of a pathogen (e.g., a virus gene or a bacteria gene) in a subject, wherein the targeting of the gene of the pathogen results in the prevention or treatment of disease in the subject. More specifically, the effector RNA may target a gene of a pathogen that is essential to replication or survival of the pathogen. For example, the effector RNA could target a bacterial hepA gene, e.g., the Shigella flexneri hepA gene (e.g., Accession Number NC_{—}008258.1), resulting in attenuation of the bacteria. As another example, the effector RNA could target the nucleoprotein (NP) of a virus, e.g., a SARS coronavirus NP (e.g., Accession Number AY291315.1) or an Influenza A virus NP (e.g., accession number EF190975.1), resulting in attenuation of the virus. In a specific embodiment, a recombinant RNA virus provides an effector RNA that targets influenza virus. The ability of miRNA for use in targeting of viruses such as SARS coronavirus, Ebola virus, H.I.V., RSV, hepatitis C virus, and influenza A virus has been demonstrated (see, e.g., Yokota et al., 2007, Biochem. Biophys. Res. Commun. 361:294-300; Kumar et al., 2008, Cell 134:577-586; Bitko et al., 2005, Nature Med. 11:50-55; Li et al., 2005, Nature Med. 11:944-951; and Tompkins et al., 2004, Proc. Natl. Acad. Sci. USA 101:8682-8686) and in specific emboidments such miRNA described in these examples can be used in accordance with the methods described herein to target such viruses.

The microRNA miR-33, in conjunction with SREBP genes, works to control cholesterol homeostasis (see, e.g., Rayner et al., 2010, Science 328:1570-1573; and Najafi-Shoushtari et al., 2010, Science 328:1566-1569). Thus, in accordance with the methods for preventing or treating disease described herein, miR-33, as well as the SREBP genes, could be could be targeted with effector RNA produced by a recombinant RNA virus described herein as a means to regulate the levels of cholesterol in an individual in need of such regulation.

Viral production of miRNA and the role of certain miRNAs in the viral life cycle have been described (see, e.g., Cullen, 2010, PLoS Pathog. 6(2):e1000787). As such, in certain embodiments, the effector RNA produced by a recombinant RNA virus described herein can be engineered to target an miRNA of a virus in a subject, such that the targeting of the miRNA of the virus results in the prevention or treatment of a disease associated with viral infection in the subject.

Certain miRNAs have been demonstrated to play a role in cancer pathogenesis (see, e.g., Kawasaki and Taira, 2003, Nature 423:838-843; He et al., 2005, Nature 435 (7043):828-833; and Mraz et al., 2009, Leuk Lymphoma 50 (3): 506-509). As such, in certain embodiments, the effector RNA produced by a recombinant RNA virus described herein can be engineered to target an miRNA involved in cancer in a subject, such that the targeting of the miRNA results in the prevention or treatment of cancer in the subject.

In specific embodiments, the heterologous RNA is transcribed to target an svRNA, such as an svRNA of influenza virus (see, e.g., Perez et al., Proc Natl Acad Sci USA 107, 11525-11530 (2010)), to treat or prevent an infection with the virus that expresses the svRNA.

In another specific embodiment, the virus targeted is a recombinant RNA virus described herein, i.e., the effector RNA targets its vector so as to attenuate and/or self-regulate the viral vector. In a specific embodiment, such self-regulation is accomplished by incorporating into the viral genome an MRE that is responsive to an effector RNA expressed the virus. This can be accomplished by inserting into the viral genome an MRE that is responsive to the effector RNA expressed by the virus such that in the presence of the miRNA to which the MRE is associated (e.g., due to production of the effector RNA by the virus), the virus is attenuated.

5.7.3 Inducing or Enhancing an Immune Response

In one aspect, the recombinant RNA viruses described herein can be used to induce or enhance an immune response in a subject. In one embodiment, the recombinant RNA viruses described herein can be used as a vaccine.

When used as vaccine, the recombinant RNA viruses described herein can vaccinate a subject against the recombinant RNA virus itself and/or one or more additional viruses by means of expressing a heterologous nucleic acid sequence that is specific to another virus and known to generate an immune response. For example, an attenuated virus (e.g., a vaccine strain), e.g., an influenza virus, could be constructed such that it produces artificial microRNA against a viral target of interest (e.g., an influenza virus or a different virus) while itself being engineered to be receptive to a particular miRNA of interest (see, e.g., Perez et al., Nat Biotechnol 27, 572-576 (2009)). Such virus could serve as both a vaccine and a viral prophylactic (see, e.g., Varble et al., 2011, RNA Biology 8:190-194). In some embodiments, a recombinant RNA virus vaccine encompassed herein comprises an effector RNA that enhances the host immune response to the vaccine by targeting a gene of the subject known to be involved in the host immune response. In some embodiments, a recombinant RNA virus vaccine encompassed herein comprises an effector RNA that targets a gene of a pathogen.

In another embodiment, the recombinant RNA viruses described herein can be used to enhance the host immune response to a vaccine, e.g., the recombinant RNA viruses described herein can be administered to a subject in conjunction with a vaccine. In accordance with such embodiments, the recombinant RNA virus comprises an effector RNA that enhances the host immune response to the vaccine by targeting a gene of the subject known to be involved in the host immune response.

Exemplary vaccines which the recombinant RNA viruses described herein can be administered with include, withour limitation: Anthrax vaccine, Adsorbed BCG vaccine, Diphtheria vaccine, Tetanus vaccine, Pertussis Vaccine, Hepatitis B vaccine, Poliovirus vaccine, Hepatitis A vaccine, Human Papillomavirus vaccine, Influenza virus vaccine (e.g., (e.g., Fluarix®, FluMist®, Fluvirin®, and Fluzone®), Japanese Encephalitis Virus vaccine, Measles Virus vaccine, MMR (Measles, Mumps and Rubella) vaccine, Rotavirus vaccine, Rubella Virus vaccine, Smallpox (Vaccinia) Vaccine, Typhoid vaccine, Varicella Virus vaccine, Yellow Fever vaccine, and Zoster vaccine.

Many genes are known to be involved in the host immune response. The recombinant RNA viruses described herein can be engineered to comprise an effector RNA that targets such genes, so as to result in enhanced benefit of another therapy (e.g., vaccine) that the subject receives or so as to achieve a desired response in the subject. For example and without limitation, the recombinant RNA viruses described herein can be engineered to produce effector RNA that targets the SOCS1 gene (GENE ID NO:8651), which functions to negatively regulate the function of signal transducer and activator of transcription 1 (STAT1); to produce effector RNA that targets the NFKBIA gene (IκBα GENE ID NO: 4792), which functions to negatively regulate the function NFκB; or to produce effector RNA that targets the IRF2 gene (GENE ID NO: 3660), which functions to negatively regulate transcriptional activation of interferons alpha and beta. An exemplary result of targeting such genes would be to enhance the response to the vaccine in the subject by prolonging the interferon response to the vaccine administered, increasing cytokine expression, and/or elevating major histocompatibility complex (MHC) expression. Such recombinant RNA viruses could be used as vaccines alone (i.e., the recombinant RNA virus would represent a vaccine that induces a greater host immune response than the vaccine not comprising effector RNA) or could be administered prior to, concurrently with, or subsequent to the administration of a separate vaccine (e.g., an influenza vaccine).

5.7.4 Combination Therapies

In various embodiments, a recombinant RNA virus described herein may be administered to a subject in combination with one or more other therapies. In some embodiments, a pharmaceutical composition comprising a recombinant RNA virus described herein may be administered to a subject in combination with one or more therapies. The one or more other therapies may be beneficial in the treatment or prevention of a disease or may ameliorate a symptom or condition associated with a disease. In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patent visit.

In certain embodiments, the one or more therapies is an anti-viral agent. Any anti-viral agents well-known to one of skill in the art may used in combination with a recombinant RNA virus or pharmaceutical composition described herein. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, peramivir, rimantadine, saquinavir, indinavir, ritonavir, alpha-interferons and other interferons, AZT, zanamivir (Relenza®), and oseltamivir (Tamiflu®). Other anti-viral agents include influenza virus vaccines, e.g., Fluarix® (GlaxoSmithKline), FluMist® (MedImmune Vaccines), Fluvirin® (Chiron Corporation), Flulaval® (GlaxoSmithKline), Afluria® (CSL Biotherapies Inc.), Agriflu® (Novartis) or Fluzone® (Aventis Pasteur).

In specific embodiments, the anti-viral agent is an immunomodulatory agent that is specific for a viral antigen, e.g., an influenza virus hemagglutinin polypeptide.

In certain embodiments, the one or more therapies is an anti-bacterial agent. Any anti-bacterial agents known to one of skill in the art may used in combination with a recombinant RNA virus or pharmaceutical composition described herein. Non-limiting examples of anti-bacterial agents include Amoxicillin, Amphothericin-B, Ampicillin, Azithromycin, Bacitracin, Cefaclor, Cefalexin, Chloramphenicol, Ciprofloxacin, Colistin, Daptomycin, Doxycycline, Erythromycin, Fluconazol, Gentamicin, Itraconazole, Kanamycin, Ketoconazole, Lincomycin, Metronidazole, Minocycline, Moxifloxacin, Mupirocin, Neomycin, Ofloxacin, Oxacillin, Penicillin, Piperacillin, Rifampicin, Spectinomycin, Streptomycin, Sulbactam, Sulfamethoxazole, Telithromycin, Temocillin, Tylosin, Vancomycin, and Voriconazole.

In certain embodiments, the one or more therapies is an anti-cancer agent. Any anti-cancer agents known to one of skill in the art may used in combination with a recombinant RNA virus or pharmaceutical composition described herein. Exemplary anti-cancer agents include: acivicin; anthracyclin; anthramycin; azacitidine (Vidaza); bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); carboplatin; chlorambucil; cisplatin; cytarabine (Ara-C); daunorubicin hydrochloride; decitabine (Dacogen); demethylation agents, docetaxel; doxorubicin; EphA2 inhibitors; etoposide; fazarabine; fluorouracil; gemcitabine; histone deacetylase inhibitors (HDACs); interleukin II (including recombinant interleukin II, or rIL2), interferon alpha; interferon beta; interferon gamma; lenalidomide (Revlimid); anti-CD2 antibodies (e.g., siplizumab (MedImmune Inc.; International Publication No. WO 02/098370, which is incorporated herein by reference in its entirety)); melphalan; methotrexate; mitomycin; oxaliplatin; paclitaxel; puromycin; riboprine; spiroplatin; tegafur; teniposide; vinblastine sulfate; vincristine sulfate; vorozole; zeniplatin; zinostatin; and zorubicin hydrochloride.

Other examples of cancer therapies include, but are not limited to angiogenesis inhibitors; antisense oligonucleotides; apoptosis gene modulators; apoptosis regulators; BCR/ABL antagonists; beta lactam derivatives; casein kinase inhibitors (ICOS); estrogen agonists; estrogen antagonists; glutathione inhibitors; HMG CoA reductase inhibitors; immunostimulant peptides; insulin-like growth factor-1 receptor inhibitor; interferon agonists; interferons; interleukins; lipophilic platinum compounds; matrilysin inhibitors; matrix metalloproteinase inhibitors; mismatched double stranded RNA; nitric oxide modulators; oligonucleotides; platinum compounds; protein kinase C inhibitors, protein tyrosine phosphatase inhibitors; purine nucleoside phosphorylase inhibitors; raf antagonists; signal transduction inhibitors; signal transduction modulators; translation inhibitors; tyrosine kinase inhibitors; and urokinase receptor antagonists.

In some embodiments, the therapy(ies) used in combination with a recombinant RNA virus or pharmaceutical composition described herein is an anti-angiogenic agent. Non-limiting examples of anti-angiogenic agents include proteins, polypeptides, peptides, conjugates, antibodies (e.g., human, humanized, chimeric, monoclonal, polyclonal, Fvs, ScFvs, Fab fragments, F(ab)₂ fragments, and antigen-binding fragments thereof) such as antibodies that specifically bind to TNF-α, nucleic acid molecules (e.g., antisense molecules or triple helices), organic molecules, inorganic molecules, and small molecules that reduce or inhibit angiogenesis. Other examples of anti-angiogenic agents can be found, e.g., in U.S. Publication No. 2005/0002934 A1 at paragraphs 277-282, which is incorporated by reference in its entirety. In other embodiments, the therapy(ies) used in accordance with the invention is not an anti-angiogenic agent.

In some embodiments, the therapy(ies) used in combination with a recombinant RNA virus or pharmaceutical composition described herein is an anti-inflammatory agent. Non-limiting examples of anti-inflammatory agents include non-steroidal anti-inflammatory drugs (NSAIDs) (e.g., celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™)), steroidal anti-inflammatory drugs (e.g., glucocorticoids, dexamethasone (DECADRON™), corticosteroids (e.g., methylprednisolone (MEDROL™)), cortisone, hydrocortisone, prednisone (PREDNISONE™ and DELTASONE™), and prednisolone (PRELONE™ and PEDIAPRED™)), anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)).

In certain embodiments, the therapy(ies) used in combination with a recombinant RNA virus or pharmaceutical composition described herein is an alkylating agent, a nitrosourea, an antimetabolite, an anthracyclin, a topoisomerase II inhibitor, or a mitotic inhibitor. Alkylating agents include, but are not limited to, busulfan, cisplatin, carboplatin, cholormbucil, cyclophosphamide, ifosfamide, decarbazine, mechlorethamine, mephalen, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine).

In some embodiments, a combination therapy comprises administration of two or more different recombinant RNA viruses described herein.

5.7.5 Patient Populations

In certain embodiments, a recombinant RNA virus or composition described herein may be administered to a naïve subject, i.e., a subject that does not have a disease. In one embodiment, a recombinant RNA virus or composition described herein is administered to a naïve subject that is at risk of acquiring a disease.

In certain embodiments, a recombinant RNA virus or composition described herein is administered to a patient who has been diagnosed with cancer, e.g., the patient has been diagnosed with leukemia, lymphoma, myeloma, bone and connective tissue sarcomas, brain cancer, breast cancer, ovarian cancer, kidney cancer, pancreatic cancer, esophageal cancer, stomach cancer, lung cancer (e.g, small cell lung cancer (SCLC), non-small cell lung cancer (NSCLC), throat cancer, and mesothelioma), and/or prostate cancer.

In certain embodiments, a recombinant RNA virus or composition described herein is administered to a patient who has been diagnosed with a respiratory disease, e.g., the patient has been diagnosed with a viral infection affecting the respiratory system, a bacterial infection affecting the respiratory system, asthma, cancer, chronic obstructive pulmonary disorder (COPD), emphysema, pneumonia, rhinitis, tuberculosis, bronchitis, laryngitis, tonsilitis, and/or cystic fibrosis.

In certain embodiments, a recombinant RNA virus or composition described herein is administered to a patient who has been diagnosed with an autoimmune disease, e.g., the patient has been diagnosed with Addison's disease, Behcet's disease, chronic active hepatitis, chronic fatigue immune dysfunction syndrome (CFIDS), chronic inflammatory demyelinating polyneuropathy, Churg-Strauss syndrome, Crohn's disease, Graves' disease, Guillain-Barre, Myasthenia Gravis, Reiter's syndrome, rheumatoid arthritis, sarcoidosis, Sjögren's syndrome, and/or systemic lupus erythematosus.

In certain embodiments, a recombinant RNA virus or composition described herein is administered to a patient who has been diagnosed with a disease associated with high cholesterol, e.g., the patient has been diagnosed with heart disease, stroke, peripheral vascular disease, diabetes, and/or high blood pressure.

In certain embodiments, a recombinant RNA virus or composition described herein is administered to a patient who has been diagnosed with a disease caused by infection with a virus, e.g., the patient has been infected by respiratory syncytial virus (RSV), influenza virus (influenza A virus, influenza B virus, or influenza C virus), human metapneumovirus (HMPV), rhinovirus, parainfluenza virus, SARS Coronavirus, human immunodeficiency virus (HIV), hepatitis virus (A, B, C), ebola virus, herpes virus, rubella, variola major, and/or variola minor.

In certain embodiments, a recombinant RNA virus or composition described herein is administered to a patient who has been diagnosed with a disease caused by infection with a bacteria, e.g., the patient has been infected by Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and/or Treponema pallidum.

In certain embodiments, a recombinant RNA virus or composition described herein is administered to a patient who has been diagnosed with a disease caused by infection with a fungus, e.g., the patient has been infected by Blastomyces, Paracoccidiodes, Sporothrix, Cryptococcus, Candida, Aspergillus, Histoplasma, Cryptococcus, Bipolaris, Cladophialophora, Cladosporium, Drechslera, Exophiala, Fonsecaea, Phialophora, Xylohypha, Ochroconis, Rhinocladiella, Scolecobasidium, and/or Wangiella.

In certain embodiments, a recombinant RNA virus or composition described herein is administered to a patient who has been diagnosed with a disease caused by infection with a yeast, e.g., the patient has been infected by Aciculoconidium, Botryoascus, Brettanomyces, Bullera, Bulleromyces, Candida, Citeromyces, Clavispora, Cryptococcus, Cystofilobasidium, Debaromyces, Debaryomyces, Dekkera, Dipodascus, Endomyces, Endomycopsis, Erythrobasidium, Fellomyces, Filobasidium, Guilliermondella, Hanseniaspora, Hansenula, Hasegawaea, Hyphopichia, Issatchenkia, Kloeckera, Kluyveromyces, Komagataella, Leucosporidium, Lipomyces, Lodderomyces, Malassezia—Mastigomyces, Metschnikowia, Mrakia, Nadsonia, Octosporomyces, Oosporidium, Pachysolen, Petasospora, Phaffia, Pichia, Pseudozyma, Rhodosporidium, Rhodotorula, Saccharomyces, Saccharomycodes, Saccharomycopsis, Schizoblastosporion, Schizosaccharomyces, Schwanniomyces, Selenotila, Sirobasidium, Sporidiobolus, Sporobolomyces, Stephanoascus, Sterigmatomyces, Syringospora, Torulaspora, Torulopsis, Tremelloid, Trichosporon, Trigonopsis, Udeniomyces, Waltomyces, Wickerhamia, Williopsis, Wingea, Yarrowia, Zygofabospora, Zygolipomyces, and/or Zygosaccharomyces.

In certain embodiments, a recombinant RNA virus or composition described herein is administered to a patient who has been diagnosed with a disease caused by infection with a parasite, e.g., the patient has been infected by Babesia, Cryptosporidium, Entamoeba histolytica, Leishmania, Giardia lamblia, Plasmodium, Toxoplasma, Trichomonas, Trypanosoma, Ascaris, Cestoda, Ancylostoma, Brugia, Fasciola, Trichinella, Schistosoma, Taenia, Cimicidae, Pediculus, and/or Sarcoptes.

In some embodiments, a recombinant RNA virus or composition described herein is administered to a patient with a disease (e.g., cancer or a respiratory disease) before symptoms of the disease manifest or before symptoms of the disease become severe (e.g., before the patient requires hospitalization). In some embodiments, a recombinant RNA virus or composition described herein is administered to a patient with a disease after symptoms of the disease manifest or after symptoms of the disease become severe (e.g., after the patient requires hospitalization).

In some embodiments, a subject to be administered a recombinant RNA virus or composition described herein is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In certain embodiments, a subject to be administered a recombinant RNA virus or composition described herein is a human adult. In certain embodiments, a subject to be administered a recombinant RNA virus or composition described herein is a human adult more than 50 years old. In certain embodiments, a subject to be administered a recombinant RNA virus or composition described herein is an elderly human subject.

In certain embodiments, a subject to be administered a recombinant RNA virus or composition described herein is a premature human infant. In certain embodiments, a subject to be administered a recombinant RNA virus or composition described herein is a human toddler. In certain embodiments, a subject to be administered a recombinant RNA virus or composition described herein is a human child. In certain embodiments, a subject to be administered a recombinant RNA virus or composition described herein is a human infant. In certain embodiments, a subject to whom a recombinant RNA virus or composition described herein is administered is not an infant of less than 6 months old. In a specific embodiment, a subject to be administered a recombinant RNA virus or composition described herein is 2 years old or younger.

In some embodiments, it may be advisable not to administer a live virus (e.g. a live recombinant RNA virus) to one or more of the following patient populations: elderly humans; infants younger than 6 months old; pregnant individuals; infants under the age of 1 years old; children under the age of 2 years old; children under the age of 3 years old; children under the age of 4 years old; children under the age of 5 years old; adults under the age of 20 years old; adults under the age of 25 years old; adults under the age of 30 years old; adults under the age of 35 years old; adults under the age of 40 years old; adults under the age of 45 years old; adults under the age of 50 years old; elderly humans over the age of 70 years old; elderly humans over the age of 75 years old; elderly humans over the age of 80 years old; elderly humans over the age of 85 years old; elderly humans over the age of 90 years old; elderly humans over the age of 95 years old; individuals with a history of asthma or other reactive airway diseases; individuals with chronic underlying medical conditions that may predispose them to severe viral infections; individuals with a history of Guillain-Barre syndrome; individuals with acute serious illness with fever; or individuals who are moderately or severely ill.

5.7.6 Plant Applications

In certain aspects, a recombinant RNA virus is derived from a plant RNA virus and contains and expresses a heterologous RNA that gives rise to an effector RNA that targets a plant gene to modulate a trait in the plant. In certain embodiments, the plant is wheat, tobacco, tea, coffee, cocoa, corn, soybean, sugar cane, and rice. In certain embodiments, the trait of the plant is resistance to adverse growth conditions, such as drought, flood, cold, hot, low or lack of light, extended periods of darkness, nutrient deprivation, or poor soil quality including sandy, rocky acidic or basic soil. In certain embodiments, targeting of a plant gene results in plants that grow faster, plants that generate more seed, plants with increased resistance to pests and microorganisms, or plants with improved taste or consistency for use as foods.

5.8 Modes of Administration

5.8.1 Routes of Delivery

A recombinant RNA virus or composition described herein may be delivered to a subject by a variety of routes. These include, but are not limited to, intranasal, intratracheal, oral, intradermal, intramuscular, intraperitoneal, transdermal, intravenous, conjunctival and subcutaneous routes. In specific embodiments, the route of administration is nasal, e.g., as part of a nasal spray. In certain embodiments, a composition is formulated for intramuscular administration. In some embodiments, a composition is formulated for subcutaneous administration. In certain embodiments, a composition is not formulated for administration by injection. In specific embodiments, a composition is formulated for administration by a route other than injection.

5.8.2 Dosage and Frequency of Administration

The amount of a recombinant RNA virus or composition which will be effective in one or more of the methods described herein will depend on the method being employed, and can be determined by standard laboratory and/or clinical techniques.

The precise dose to be employed in the formulation will also depend on the route of administration as well as other conditions, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but nonhuman mammals including transgenic mammals (e.g., transgenic mice) also can be treated. Treatment dosages are optimally titrated to optimize safety and efficacy.

In certain embodiments, an in vitro assay is employed to help identify optimal dosage ranges. Effective doses may be extrapolated from dose response curves derived from in vitro or animal model test systems.

Doses for recombinant RNA viruses may vary from 10-100, or more, virions per dose. In some embodiments, suitable dosages of a recombinant RNA virus are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and can be administered to a subject once, twice, three or more times with intervals as often as needed.

In certain embodiments, a recombinant RNA virus or composition is administered to a subject once as a single dose. In certain embodiments, a recombinant RNA virus or composition is administered to a subject as a single dose followed by a second dose 1 day, 3 days, 5 days, 1 week, 2 weeks, 3 weeks, 4 weeks, one month, two months, three months, four months, five months, 6 months, or 1 year later. In some embodiments, the administration of a recombinant RNA virus or composition may be repeated for a specified time period and the administrations may be separated by at least 1 day, 2 days, 3 days, 5 days, 10 days, 15 days, 30 days, 45 days, 2 months, 75 days, 3 months, or at least 6 months. In certain embodiments, a recombinant RNA virus or composition is administered to a subject as a single dose once, twice, or three times per year.

5.9 Assays

5.9.1 Assays for Testing Virus Replication

The ability of a recombinant RNA virus described herein to effectively replicate while expressing an effector RNA can be assessed using methods known to those of skill in the art and described in Section 6, infra. For example, methods such as multi-cycle growth curves can be utilized to determine the replicative capacity of recombinant RNA viruses that express effector RNA. Briefly, cells are infected with a recombinant RNA virus that expresses effector RNA followed by removal of virus-containing supernatant at various time points. The supernatant is then used in a plaque assay and plaques, indicative of the number of recombinant RNA viruses present, are counted.

The rate of replication of the recombinant viruses described herein can be determined by any standard technique known to the skilled artisan. The rate of replication is represented by the growth rate of the virus and can be determined by plotting the viral titer over the time post-infection. The viral titer can be measured by any technique known to the skilled artisan. In certain embodiments, to measure viral titer, a suspension containing the recombinant RNA virus is incubated with cells that are susceptible to infection by the virus. Cell types that can be used with the methods of the invention include, but are not limited to, Vero cells, LLC-MK-2 cells, Hep-2 cells, LF 1043 (HEL) cells, MRC-5 cells, WI-38 cells, 293 T cells, QT 6 cells, QT 35 cells, chicken embryo fibroblast (CEF), or tMK cells. Subsequent to the incubation of the recombinant RNA virus with the cells, the number of infected cells is determined. In certain specific embodiments, the recombinant RNA virus comprises a reporter gene. Thus, the number of cells expressing the reporter gene is representative of the number of infected cells. In a specific embodiment, the recombinant RNA virus comprises a heterologous nucleotide sequence encoding for eGFP, and the number of cells expressing eGFP, i.e., the number of cells infected with the recombinant RNA virus, is determined using FACS.

5.9.2 Assays for Testing Effector RNA Processing

5.9.2.1 Drosha Assays

The ability of Drosha to process heterologous RNA can be assessed using any assay known in the art. Exemplary assays for assessing Drosha processing are described in Zeng and Cullen, 2005, J. Biol. Chem. 280(30):27595-27603. In certain embodiments, an enzymatic assay can be performed to assess Drosha processing of heterologous RNA. Briefly, purified Drosha is mixed with radiolabeled heterologous RNA comprising effector and incubated for 60-90 minutes at 37° C. After terminating the enzymatic reaction, the ability of Drosha to process the heterologous RNA, thus resulting in the generation of precursor effector RNA (e.g., pri-miRNAs) can then be assessed by Northern blot analysis (see Zeng and Cullen, 2005, J. Biol. Chem. 280(30):27595-27603).

5.9.2.2 Drosha/DGCR8 Complex Assays

The ability of the Drosha/DGCR8 complex to process heterologous RNA can be assessed using any assay known in the art. In certain embodiments, an immunoprecipitation assay can be performed to test Drosha/DGCR8 processing. For example, either Drosha or DGCR8 can be immunoprecipitated and incubated with a radiolabeled (e.g., with alpha P32 UTP) T7-transcribed pre-microRNA. Immunoprecipitated extract can then be incubated with the synthetic RNA hairpin for one hour at 37° C. In vitro processing can then be measured by stardard gel electrophoresis (see, e.g., Zeng and Cullen, 2005, J. Biol. Chem. 280(30):27595-27603; and Lee et al. Methods Enzymol. 2007, 427:89-106).

5.9.2.3 Dicer Assays

The ability of Dicer to process heterologous RNA can be assessed using any assay known in the art. Dicer processing can be assessed using assays similar to those described in Section 5.10.3.1 for assessing Drosha RNA processing, e.g., enzymatic assays can be performed to test the ability of purified Dicer to process heterologous RNA. Additional assays for assessing Dicer processing have been described in DiNitto et al., 2010, BioTechniques 48(4):303-311. In certain embodiments, the ability of Dicer to process heterologous RNA can be assessed using a fluorgenic Dicer assay. Briefly, fluorescently-labeled heterologous RNA to be used as Dicer substrate is generated that possesses a quencher moiety (e.g., Iowa Black RQ; IDT, Coralville, Iowa) which quenches fluorescence of the heterologous RNA when the heterologous RNA has not been processed by Dicer. The fluorescently-labeled heterologous RNA is incubated with increasing concentrations of purified Dicer at 30° C., with change in fluorescence measured over time as Dicer concentration increases. Dicer processing of the heterologous RNA results in release of the quencher moiety and a measurable incease in fluorescence intensity (see, e.g., DiNitto et al., 2010, BioTechniques 48(4):303-311).

5.9.2.4 Exportin 5 Assays

The ability of exportin 5 to bind a primary transcript from heterologous RNA (e.g., after Drosha processing) can be assessed using any assay known in the art. Exemplary approaches for assessing exportin 5 binding have been described in Brownawell et al., 2002, J. Cell Biol. 156(1):53-64. Briefly, labeled primary transcript from heterologous RNA is bound to beads (e.g., Protein A Sepharose or GSH beads) and incubated with labeled (e.g., HIS-Tag) exportin 5. Following incubation, the beads are suspended in Laemmli sample buffer, separated by SDS-PAGE, and analyzed by Western blot. The presence of both exportin 5 and the primary transcript from heterologous RNA as revealed by chemiluminescence is indicative of exportin 5 binding (see Brownawell et al., 2002, J. Cell Biol. 156(1):53-64).

The ability of exportin 5 to export primary transcript from heterologous RNA from the nucleus to the cytoplasm can be assessed using any assay known in the art (see, e.g., Brownawell et al., 2002, J. Cell Biol. 156(1):53-64).

5.9.2.5 Dicer/TRBP/PACT Complex Assays

The ability of the Dicer/TRBP/PACT complex to process heterologous RNA can be assessed using any assay known in the art. In certain embodiments, the ability of the Dicer/TRBP/PACT complex to process heterologous RNA can be assessed using the approaches described in Section 5.9.2.2.

5.9.3 Assays for Testing Expression of Effector RNA

The ability of a recombinant RNA virus described herein to express an effector RNA can be assessed using methods known to those of skill in the art and described in Section 6, infra. Exemplary approaches for assessing expression of effector RNA include Northern blot analysis (see, e.g., Pall and Hamilton, 2008, Nat. Protoc. 3(6):1077-1084); stem-loop-specific quantitative PCR (see, e.g., Chen et al., 2005, Nucleic Acids Res. 33(20):e179); and RNase protection assay (RPA) (see, e.g., Gillman et al, Curr Protoc Mol Biol 2001, Unit 4.7).

5.9.4 Assays for Testing Effect on Target Genes

The ability of an effector RNA produced by a recombinant RNA virus described herein to modulate target gene expression can be assessed using methods known to those of skill in the art and described in Section 6, infra. In certain embodiments, the ability of an effector RNA produced by a recombinant RNA virus described herein to modulate target gene expression can be assessed using assays that detect RNA expression or by using assays that detect protein expression. Exemplary approaches for assessing expression of RNA include Northern blot analysis (see, e.g., Pall and Hamilton, 2008, Nat. Protoc. 3(6):1077-1084); stem-loop-specific quantitative PCR (see, e.g., Chen et al., 2005, Nucleic Acids Res. 33(20):e179); and RNase protection assay (RPA) (see, e.g., Gillman et al, Curr Protoc Mol Biol 2001, Unit 4.7). Exemplary approaches for assessing expression of protein include Western blot and enzyme-linked immunosorbent assays (ELISA).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive isotope (e.g., ³²P or ¹²⁵I)-labeled molecule diluted in blocking buffer, washing the membrane in wash buffer, and detecting the presence of the antigen. One of skill in the art would be knowledgeable as to the experimental variables that can be modified to increase the signal detected and to reduce the background signal.

ELISAs generally comprise preparing a solution of the antigen (for example, a cell lysate containing the antigen of interest or a buffered solution of a purified antigen of interest), coating the wells of a 96 well microtiter plate with the antigen, washing the wells with an inert buffer solution, adding an antigen-recognizing antibody conjugated to a reporter compound such as an enzymatic reporter (e.g., horseradish peroxidase or alkaline phosphatase) to the wells, incubating for a period of time, removing the excess conjugated antibody, washing the wells extensively with an inert buffer solution, and measuring the amount or the activity of retained reporter. In ELISAs, the antibody of interest does not have to be conjugated to a reporter compound; instead, a second antibody (which specifically binds the antigen-recognizing antibody) conjugated to a reporter compound may be added to the wells. Further, instead of coating the wells with the antigen, the antibody may be coated to the wells first. In this case, a second antibody conjugated to a reporter compound may be added following the addition of the antigen of interest to the coated wells. The antibody of interest does not have to be conjugated to a reporter compound; instead, a second antibody (which specifically binds the antigen-recognizing antibody) conjugated to a reporter compound may be added to the wells. One skilled in the art would be knowledgeable as to the experimental variables that can be modified to increase the signal detected as well as other variations of ELISAs known in the art.

5.9.5 Antiviral Activity Assays

Effector RNA expressed by a recombinant RNA virus described herein or compositions thereof can be assessed in vitro for antiviral activity. In one embodiment, the effector RNA tested in vitro for its effect on growth of a virus, e.g., an influenza virus. Growth of virus can be assessed by any method known in the art or described herein (see, e.g., Section 5.10.2). In a specific embodiment, cells are infected with a recombinant RNA virus at a MOI of 0.0005 and 0.001, 0.001 and 0.01, 0.01 and 0.1, 0.1 and 1, or 1 and 10, or a MOI of 0.0005, 0.001, 0.005, 0.01, 0.05, 0.1, 0.5, 1, 5 or 10. Viral titers are determined in the supernatant by plaque assay or any other viral assay described herein. In vitro assays include those that measure altered viral replication (as determined, e.g., by plaque formation) or the production of viral proteins (as determined, e.g., by Western blot analysis) or viral RNAs (as determined, e.g., by RT-PCR or northern blot analysis) in cultured cells in vitro using methods which are well known in the art or described herein.

5.9.6 Cytotoxicity Assays

Many assays well-known in the art can be used to assess viability of cells (infected or uninfected) or cell lines following exposure to a recombinant RNA virus or a composition thereof and, thus, determine the cytotoxicity of the recombinant RNA virus or composition. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). Cell viability can also be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic—very heavy—80%), PH (partially toxic—heavy—60%), P (partially toxic—40%), Ps (partially toxic—slight—20%), or 0 (no toxicity—0%), conforming to the degree of cytotoxicity seen. A 50% cell inhibitory (cytotoxic) concentration (IC₅₀) is determined by regression analysis of these data.

In a specific embodiment, the cells used in the cytotoxicity assay are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells. In certain embodiments, cytotoxicity is assessed in one or more of the following cell lines: MDCK, MEF, Huh 7.5, Detroit, or human tracheobronchial epithelial (HTBE) cells.

Recombinant RNA viruses or compositions thereof can be tested for in vivo toxicity in animal models. For example, animal models, described herein and/or others known in the art, used to test the activities of viruses can also be used to determine the in vivo toxicity of the recombinant RNA viruses described herein. For example, animals are administered a range of concentrations of recombinant RNA viruses. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of a recombinant RNA virus can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A recombinant RNA virus that exhibits large therapeutic indices is preferred. While a recombinant RNA virus that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of a recombinant RNA virus for use in humans. The dosage of such recombinant RNA viruses lies preferably within a range with little or no toxicity. The dosage may vary within this range depending upon the dosage form employed and the route of administration utilized. For any recombinant RNA virus used in a method described herein, the effective dose can be estimated initially from cell culture assays. Additional information concerning dosage determination is provided herein.

Further, any assays known to those skilled in the art can be used to evaluate the prophylactic and/or therapeutic utility of the recombinant RNA viruses and compositions described herein.

5.9.7 In Vivo Activity in Non-Human Animals

Recombinant RNA viruses and compositions thereof are preferably assayed in vivo for the desired therapeutic or prophylactic activity prior to use in humans. For example, in vivo assays using non-human animals as models can be used to determine whether it is preferable to administer a recombinant RNA virus or composition thereof and/or another therapy.

Recombinant RNA viruses and compositions thereof can be tested for activity in animal model systems including, but are not limited to, rats, mice, chicken, cows, monkeys, pigs, goats, sheep, dogs, rabbits, guinea pigs, etc. In a specific embodiment, recombinant RNA viruses and compositions thereof are tested in a mouse model system. Such model systems are widely used and well-known to the skilled artisan.

In general, non-human animals serving as a model for disease are treated with a recombinant RNA virus or composition thereof, or placebo. Subsequently, the animals may be monitored for disease status and progression and the ability of the recombinant RNA virus to prevent and/or treat the disease can be assessed. In certain embodiments, histopathologic evaluations are performed to assess the effect of the recombinant RNA virus. Tissues and organs of the animal treated with a recombinant RNA virus may be assessed using approaches known to those of skill in the art.

5.9.7.1 Anti-Cancer Studies

The recombinant RNA viruses described herein or pharmaceutical compositions thereof can be tested for biological activity using animal models for cancer. Such animal model systems include, but are not limited to, rats, mice, chicken, cows, monkeys, pigs, dogs, rabbits, etc. In a specific embodiment, the anti-cancer activity of a recombinant RNA virus described herein is tested in a mouse model system. Such model systems are widely used and well-known to the skilled artisan such as the SCID mouse model or transgenic mice.

The anti-cancer activity of a recombinant RNA virus described herein or a pharmaceutical composition thereof can be determined by administering the recombinant RNA virus or pharmaceutical composition thereof to an animal model and verifying that the recombinant RNA virus or pharmaceutical composition thereof is effective in reducing the severity of cancer in said animal model. Examples of animal models for cancer in general include, include, but are not limited to, spontaneously occurring tumors of companion animals (see, e.g., Vail & MacEwen, 2000, Cancer Invest 18(8):781-92). Examples of animal models for lung cancer include, but are not limited to, lung cancer animal models described by Zhang & Roth (1994, In-vivo 8(5):755-69) and a transgenic mouse model with disrupted p53 function (see, e.g. Morris et al., 1998, J La State Med Soc 150(4): 179-85). An example of an animal model for breast cancer includes, but is not limited to, a transgenic mouse that over expresses cyclin D1 (see, e.g., Hosokawa et al., 2001, Transgenic Res 10(5):471-8). An example of an animal model for colon cancer includes, but is not limited to, a TCR b and p53 double knockout mouse (see, e.g., Kado et al., 2001, Cancer Res. 61(6):2395-8). Examples of animal models for pancreatic cancer include, but are not limited to, a metastatic model of Panc02 murine pancreatic adenocarcinoma (see, e.g., Wang et al., 2001, Int. J. Pancreatol. 29(1):37-46) and nu-nu mice generated in subcutaneous pancreatic tumors (see, e.g., Ghaneh et al., 2001, Gene Ther. 8(3):199-208). Examples of animal models for non-Hodgkin's lymphoma include, but are not limited to, a severe combined immunodeficiency (“SCID”) mouse (see, e.g., Bryant et al., 2000, Lab Invest 80(4):553-73) and an IgHmu-HOX11 transgenic mouse (see, e.g., Hough et al., 1998, Proc. Natl. Acad. Sci. USA 95(23):13853-8). An example of an animal model for esophageal cancer includes, but is not limited to, a mouse transgenic for the human papillomavirus type 16 E7 oncogene (see, e.g., Herber et al., 1996, J. Virol. 70(3):1873-81). Examples of animal models for colorectal carcinomas include, but are not limited to, Apc mouse models (see, e.g., Fodde & Smits, 2001, Trends Mol Med 7(8):369 73 and Kuraguchi et al., 2000).

5.9.8 Assays in Humans

In one embodiment, the ability of a recombinant RNA virus or composition thereof to prevent or treat disease is assessed in human subjects having a disease. In accordance with this embodiment, a recombinant RNA virus or composition thereof is administered to the human subject, and the effect of the recombinant RNA virus or composition on the disease is determined.

In another embodiment, the ability of a recombinant RNA virus or composition thereof to reduce the severity of one or more symptoms associated with a disease is assessed in having a disease. In accordance with this embodiment, a recombinant RNA virus or composition thereof or a control is administered to a human subject suffering from a disease and the effect of the recombinant RNA virus or composition on one or more symptoms of the disease is determined. A recombinant RNA virus or composition thereof that reduces one or more symptoms can be identified by comparing the subjects treated with a control to the subjects treated with the recombinant RNA virus or composition. Techniques known to physicians familiar with the disease can be used to determine whether a recombinant RNA virus or composition thereof reduces one or more symptoms associated with the disease.

In another embodiment, a recombinant RNA virus or composition thereof is administered to a healthy human subject and monitored for efficacy as a vaccine. Techniques known to physicians familiar with infectious diseases can be used to determine whether a recombinant RNA virus or composition thereof is effective as a vaccine.

5.10 Kits

Provided herein is a pharmaceutical pack or kit comprising one or more containers filled with one or more of the ingredients of the pharmaceutical compositions described herein, such as one or more recombinant RNA viruses provided herein. The kits provided herein also may comprise one or more recombinant RNA viruses provided herein, i.e., the recombinant RNA viruses in the kit are not formulated as a pharmaceutical composition but rather are formulated for experimentation purposes. Also provided herein are kits comprising one or more of the chimeric viral genomic segments or chimeric genes described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in the above methods. In one embodiment, a kit comprises a recombinant RNA virus described herein. In a specific embodiment, a kit comprises a recombinant influenza virus. In another specific embodiment, a kit comprises a recombinant sindbis virus. In another specific embodiment, a kit comprises one or more of the chimeric viral genomic segments or chimeric genes described herein.

### 6. EXAMPLES

### 6.1 Example 1

This example demonstrates that influenza virus can be engineered to produce functional miRNA without loss of viral growth.

6.1.1 Materials and Methods

6.1.1.1 Cell Culture

HEK293, MDCK, CAD, and murine fibroblasts were cultured in DMEM (Mediatech) media supplemented with 10% Fetal Bovine Serum and 1% penicillin/streptomycin. Dicer deficient fibroblasts were provided by A. Tarakhovsky (Rockefeller University, NYC) and Donal O'Carrol (EMBL, Monterotondo) and CAD cells were provided by T. Maniatis (Columbia University, NYC).

6.1.1.2 Virus Design and Rescue

The modified NS segment (A/PR/8/34) was generated by PCR, followed by a three-way ligation. The splice acceptor site in the NS1 ORF (521 5′ tcttccaggacat3′ 533) was mutated to prevent splicing (521 5′ tctCccGggacat3′ 533) of NS mRNA at this site by site-directed mutagenesis using the primers 5′-CCATTGCCTTCTCTCCCGGGACATACTGCTGAGGATGTC-3′ (SEQ ID NO:5) and 5′-GACATCCTCAGCAGTATGTCCCGGGAGAGAAGGCAATGG-3′ (SEQ ID NO:6). The fragment corresponding to the NS1 ORF along with the 3′ non-coding region of vRNA (1-716 nucleotides) was amplified from this splice acceptor site mutant NS segment with primers carrying SapI and XhoI site (5′-GATCGCTCTTCTGGGAGCAAAAGCAGG-5′ (SEQ ID NO:7) and 5′-CCCCTCGAGTCAAACTTCTGACCTAATTGTTCCC-5′ (SEQ ID NO:8)). The fragment corresponding to the NEP/NS2 and 5′-noncoding region of vRNA (nucleotide 508-890 in the Wt NS segment) was amplified from a NS plasmid using primers carrying XhoI and SapI sites (5′-CGCTCGAGCACCATTGCCTTCTCTTCCAGG-3′ (SEQ ID NO:9) and 5′-CATCGCTCTTCTATTAGTAGAAACAAGG-3′ (SEQ ID NO:10)). The NS1 and NEP/NS2 fragments were digested with SapI and XhoI, and ligated into a pDZ rescue vector cut with SapI. The recombinant viruses were rescued by using previously described reverse genetic techniques (see, e.g., Hoffmann et al. (2000) Proc Natl Acad Sci USA 97(11):6108-6113; and Fodor et al. (1999) J Virol 73(11):9679-9682). Briefly, 0.5 μg of each of the 8 pDZ plasmids representing the 8-segments of IAV genome were transfected into 293T cells. After 24 h, the 293T cells with supernatants were injected into 8-day old eggs. The recombinant virus was harvested from the allantoic fluid at 48 hours post infection. After plaque purification, the modified NS segment was confirmed by sequencing the RT-PCR product of vRNA. A ClaI restriction site was further introduced into the intergenic region of the NS vRNA by performing standard site directed mutagenesis. The ClaI insertion site was used to ligate the miR-124-2 murine locus (chr3:17,695,454-17,696,037) or four copies of miR-142-3p targets as previously described (see, e.g., Brown et al. (2007) Nat Biotechnol 25(12):1457-1467).

6.1.1.3 Virus Infections

Viral infections were performed at the multiplicity of infections (MOIs) specified. Virus was inoculated into indicated cell lines containing phosphate buffered saline (PBS) media supplemented with 0.3% Bovine Serum Albumin (BSA, MP Biomedicals) and penicillin/streptomycin for 1 hour. Inoculum then was aspirated off and replaced with either fresh complete medium for the indicated times or in minimal essential media supplemented with 0.5 or 5% BSA and L-(tosylamido-2-phenyl)ethyl chloromethyl ketone (TPCK) trypsin.

6.1.1.4 Northern Blot Analysis

Northern blots and probe labeling were performed as previously described (see, e.g., Pall and Hamilton (2008) Nat Protoc 3(6):1077-1084). Probes used include: anti-miR-124: 5′-TGGCATTCACCGCGTGCCTTAA-3′ (SEQ ID NO:11), anti-miR-93: 5′-CTACCTGCACGAACAGCACTTTG-3′ (SEQ ID NO:12), miR-142-3p: 5′-TCCATAAAGTAGGAAACACTACA-3′ (SEQ ID NO:13) and anti-U6: 5′-GCCATGCTAATCTTCTCTGTATC-3′ (SEQ ID NO:14).

6.1.1.5 Western Blot Analysis

Western blots were generated from total protein separated on a 15% SDS-PAGE gel. Resolved protein was transferred to nitrocellulose (Bio-Rad), blocked for 1 hour with 5% skim milk at 25° C. and then incubated with the indicated antibody overnight at 4° C. Actin (Abcam), NS1, NEP/NS2, and NP (provided by P. Palese, MSSM, NYC) antibodies were all used at a concentration of 1 microgram/ml in 5% skim milk. Secondary mouse and rabbit antibodies (GE Healthcare) were used at a 1:5000 dilution for 1 hour at 25° C. Immobilon Western Chemiluminescent HRP Substrate (Millipore) was used as directed.

6.1.1.6 Immunofluoresence

Cells were fixed on glass coverslips by incubating with 4% formaldehyde overnight at 4° C. Following two PBS washes, cells were permeabilized with 0.5% NP40 detergent in PBS for 10 min and immediately washed two additional times. The cells then were blocked with a 0.5% bovine albumin solution (BSA) in PBS for 30 min. at room temperature. Primary antibody was incubated for 2 h at room temperature at a 1:500 concentration. The monoclonal antibody (E7-β-tubulin) was obtained from the Developmental Studies Hybridoma Bank Following four washes in 0.5% BSA in PBS, cells were incubated with secondary antibody, Rhodamine Red-X (Fisher), at 1:750 for 1 hr with Hoechst 33342 dye (Invitrogen) added with 15 minutes remaining. Following four washes, coverslips were mounted on glass slides with Prolong Gold Antifade (Invitrogen). Images were captured with the Leica TCS SP5 DMI microscope at 60× magnification.

6.1.1.7 Quantitative PCR

Conventional Quantitative PCR was performed on the indicated cDNA samples using KAPA SYBR® FAST qPCR Master Mix (KAPA Biosystems) and microRNA Quantitative PCR was performed using TaqMan® MicroRNA Assays (Applied Biosystems). Experiments were performed on Mastercycler ep realplex (Eppendorf). ΔΔCT values were calculated over replicates using tubulin or snoRNA 202 as the endogenous housekeeping gene and mock-infected or mock-transfected samples as the calibrator in respective experiments. Values represent the fold difference for each condition as compared to mock-infected or transfected samples. Error bars reflect +/−standard deviation of fold induction. Primers used for QRT-PCR are described below.

6.1.1.8 5′ RACE.

5′RACE was performed on virally-infected samples using 5′ RACE System for Rapid Amplification of cDNA ends, Version 2.0 (Invitrogen). The procedure was carried out according to manufacturer's instructions. In brief, first strand cDNA synthesis was performed using viral cRNA specific primer, 5′-AGTAGAAACAAGGGTGTTTTTTAT-3′ (SEQ ID NO:15). cDNA was purified using S.N.A.P. purification columns, then tailed with dCTP using TdT. The cDNA then was amplified using EconoTaq (VWR) with the provided Abridged Anchor primer and nested NEP primer, 5′-AATGGATCCAAACACTGTGTCA-3′ (SEQ ID NO:16). Fragments then were gel purified using QIAquick® Gel extraction kit (Qiagen) and cloned for sequencing using TOPO TA Cloning® kit (Invitrogen).

6.1.1.9 Multicycle Growth Curve

MDCK cells were infected with viruses indicated at an MOI 0.01. 225 μl of supernatant was removed at the indicated times. Supernatant then was plagued in MDCK cells in serial dilutions in triplicate in an MEM-agar overlay supplemented with 0.01% DEAE-Dextran (Sigma) and 0.1% NaHCO₃ (Sigma). Plaques were counted after 2 days post-infection.

6.1.1.10 FACS

GFP_miR-124t was generated by synthesizing and inserting four, perfectly complementary, miR-124 target sites into the pEGFPC1 plasmid (Genebank accession # U55763) via HindIII and BamHI restriction enzymes. FACS analysis was performed on 2×10e6 cells/ml resuspended in PBS with 2% FBS. GFP expression was quantified through the FL1 channel with the Cytomics Fc 500 (Beckman) instrument.

6.1.1.11 qPCR Primers

qPCR and RT primers used include: PB2 5′-ATCGGAATCGCAACTAACGA-3 (SEQ ID NO:17) and 5′-TTTGCGGACCAGTTCTCTCT-3′ (SEQ ID NO:18). Canine tubulin 5′-GGTTCGAGTTCTGGAAGCAG-3′ (SEQ ID NO:19) and 5′-GGGGATGTAGTGCTCATCGT-3′ (SEQ ID NO:20), NEP/NS2 5′-CACTGTGTCAAGCTTTCAGGACATACTG-3′ (SEQ ID NO:21) and 5′-CTCGTTTCTGTTTTGGAGTGAGTG-3′ (SEQ ID NO:22), NS1 (for standard RT) 5′-GGGCTTTCACCGAAGAGGGAGC-3′ (SEQ ID NO:23) and 5′-GTGGAGGTCTCCCATTCTCA-3′ (SEQ ID NO:24), NS 5′ cRNA 5′-GACCAAGAACTAGGCGATGC-3′ (SEQ ID NO:25) and 5′-CGCTCCACTATCTGCTTTCC-3′ (SEQ ID NO:26), NS 3′ cRNA 5′-AAGGGTGAGACACAAACTGAAGGT-3′ (SEQ ID NO:27) and 5′-AGTAGAAACAAGGGTGTTTTTTAT-3′ (SEQ ID NO:28), and NS loop 5′-CCATCGATGAGCTCCAAGAGAGGGTGAA-3′ (SEQ ID NO:29) and 5′-CCATCGATTCTCCCCACCCTTCCTAACT-3′ (SEQ ID NO:30), murine tubulin 5′-TGCCTTTGTGCACTGGTATG-3′ (SEQ ID NO:31) and 5′-CTGGAGCAGTTTGACGACAC-3′ (SEQ ID NO:32).

6.1.2 Results

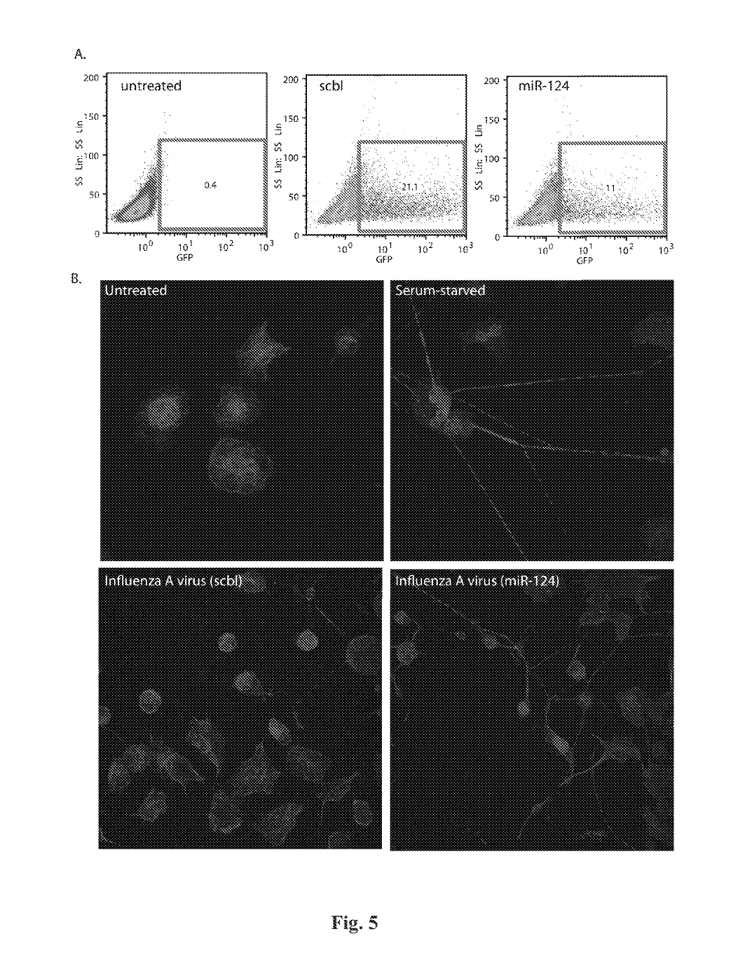

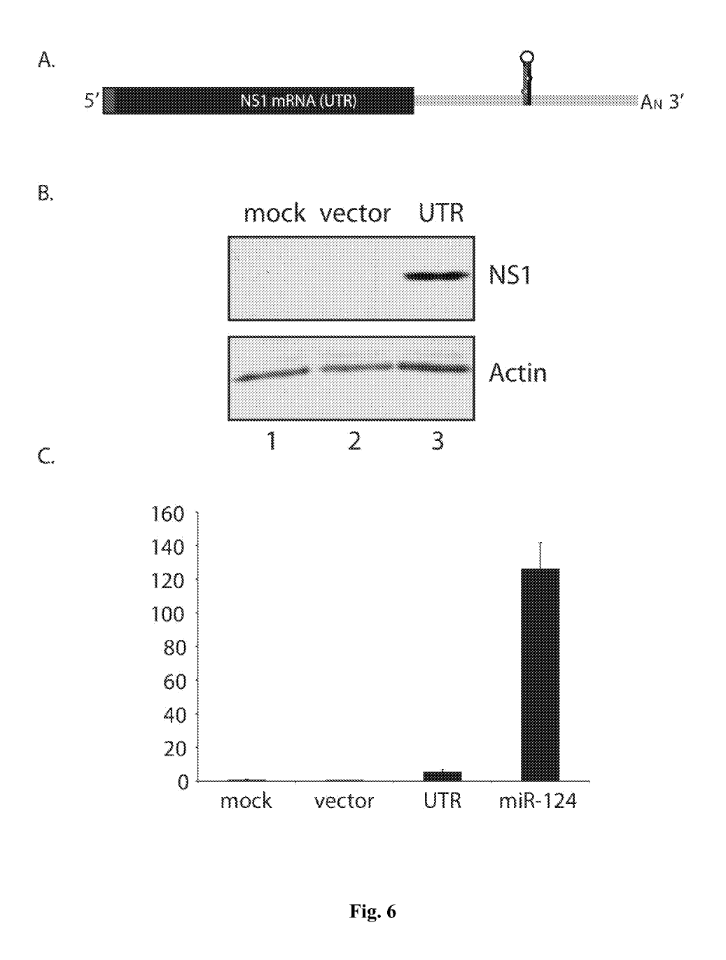

An influenza A virus was engineered to encode a known microRNA locus and the impact on miRNA processing, PTGS activity, and virus replication was ascertained. As two of the eight negative stranded segments that compose the genome of influenza A virus undergo splicing during infection (see, e.g., Palese and Shaw (2007) in Fields Virology 5th Edition, eds Knipe, D M and Howley, P M. (Raven, Philadelphia), pp 1648-1698), whether the virus would permit the insertion of a mammalian pri-miRNA in the context of a viral intron, thereby mimicking a number of well characterized endogenous miRNAs (see, e.g., Kim and Kim (2007) EMBO J 26(3):775-783) was investigated. To perform these studies segment eight was chosen because it is the shorter of two viral transcripts that undergo splicing, and it was believed that it thus would be more amenable to the addition of genetic material. Segment eight encodes two proteins, the non-structural protein 1 (NS1) which confers a block on cellular antiviral activity (see, e.g., Salvatore et al. (2002) J Virol 76(3):1206-1212), and the nuclear export protein (NEP, also referred to as NS2) which is responsible for shuttling the mature RNP complexes to the cytoplasm prior to viral egress and has been implicated in controlling virus replication (see, e.g., O'Neill et al. (1998) EMBO J 17(1):288-296; and Robb et al. (2009) J Gen Virol 90(Pt 6):1398-1407). As the mRNA encoding the N-terminal of NEP/NS2 overlaps with the C-terminal transcript of NS1, the endogenous splice acceptor site was disrupted and recreated beyond the stop codon of NS1 (FIG. 1A). Synthesis of this non-overlapping split ORF created an intergenic region within segment eight that extended the 3′ UTR of NS1 and the spliced lariat of NEP/NS2. To determine whether the virus would permit insertion of a cellular pri-miRNA, either of a scrambled (scbl) genomic sequence or the murine miR-124-2 locus (in both 5′ to 3′ (miR-124) and 3′ to 5′ (miR-124(R)) orientations) were cloned into the intergenic region of segment eight and virus was generated through use of the plasmid-based rescue system (see, e.g., Hoffmann et al. (2000) Proc Natl Acad Sci USA 97(11):6108-6113; and Fodor et al. (1999) J Virol 73(11):9679-9682). Purified virus was propagated in 10-day old embryonated chicken eggs, growing to titers of approximately 10e8 to 10e9 plaque forming units (pfu) per milliliter (pfu/mL). Scbl, miR-124 and miR-124(R) fragments were additionally cloned into the intergenic region of a Red Fluorescent Protein (RFP) expressing plasmid (pRFP) as previously described (see, e.g., Makeyev et al. (2007) Mol Cell 27(3):435-448). Virus-dependent miR-124 synthesis was observed at comparable levels to transfected plasmid-based miR-124 production (FIG. 1B). Moreover, miR-124 expression was restricted by the orientation of the pre-miRNA, demonstrating expression only in its endogenous 5′ to 3′ orientation. Furthermore, miR-124 expression required splicing of NEP/NS2, as a construct only expressing NS1 with a miR-124 hairpin in the 3′ UTR, failed to produce the small RNA (FIG. 6). In addition, despite the extension of the NS1 3′ UTR and intron length of NEP/NS2, neither the insertion of scrambled sequence, nor the pri-miR-124, impacted viral protein expression as demonstrated by robust levels of nucleoprotein (NP), encoded on segment 5, and NS1 or NEP/NS2, both encoded on segment 8 (FIG. 1C). To determine the replicative capacity of the NS recombinant viruses, a multi-cycle growth curve was performed (FIG. 1D). Replication of the recombinant NS viruses demonstrated robust growth and no significant decrease in viral titers compared to wild type (wt) influenza A/PR/8/34 virus.

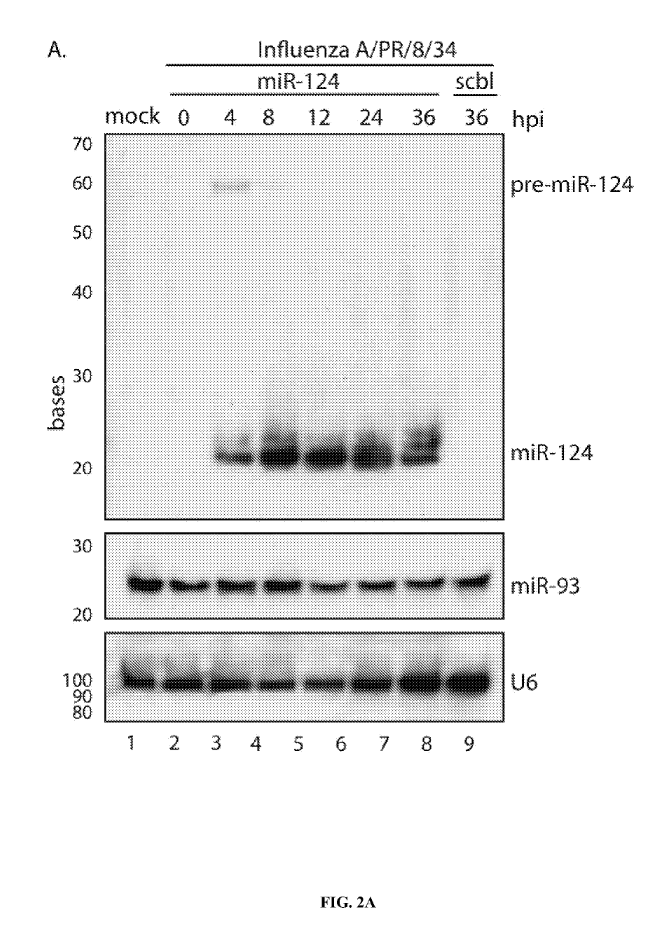

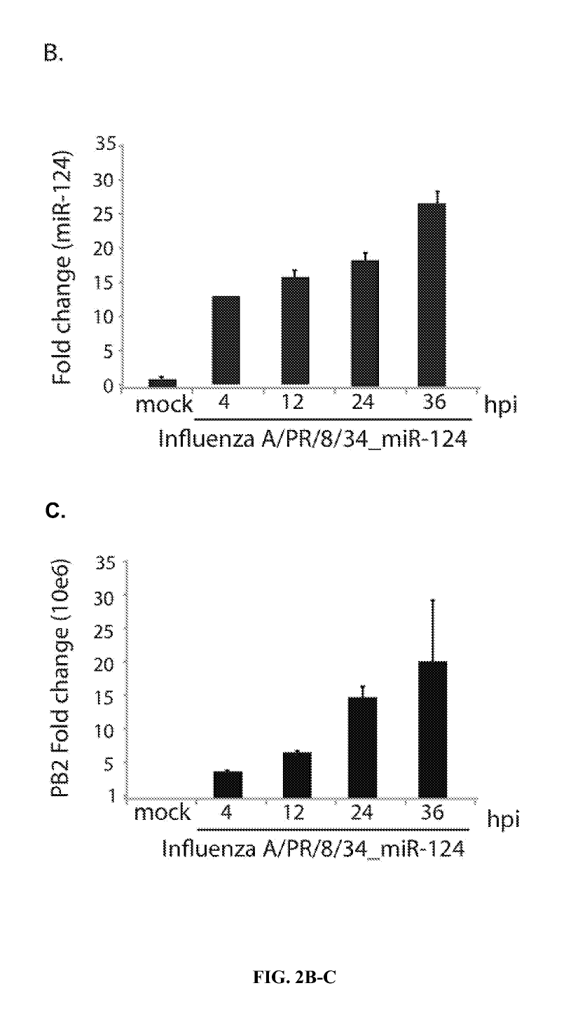

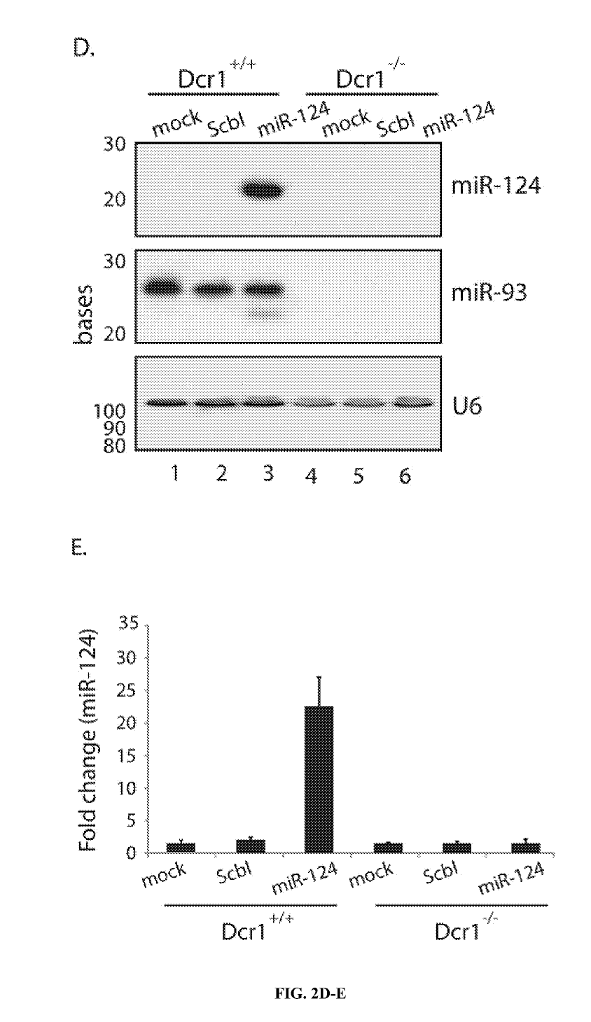

To determine whether the lariat, containing the pri-miRNA generated during NEP/NS2 synthesis, would be continually processed by the endogenous cellular machinery, miR-124-containing influenza A virus infections were performed in Madin-Darby canine kidney (MDCK) cells and were harvested at multiple time points. Small RNA Northern blots for viral-produced miR-124 demonstrated substantial expression of the miRNA as early as 4 hours post infection (FIG. 2A). The robust expression of viral-miR-124 was sustained for the duration of infection at levels comparable to that observed for endogenous miR-93. Furthermore, while pre-miR-124 was evident at 4 hours post infection, its absence at later times indicates that viral production of miRNA was not overwhelming the cell's export machinery, a phenomenon previously reported for adenovirus delivery of miRNAs (see, e.g., Grimm et al. (2006) Nature 441(7092):537-541). To ensure that the processing of pre-miR-124 mimicked the endogenous Dicer end-product, real-time quantification of miR-124 by stem-loop specific RT-PCR was performed (see, e.g., Chen et al. (2005) Nucleic Acids Res 33(20):e179). As this assay is specific for the 3′ ends of mature miRNAs and discriminates among related miRNAs that differ by as little as a single nucleotide, the robust 25-fold induction observed in response to the engineered miR-124-containing virus demonstrates that the mature product is likely a perfect mimetic of endogenous miR-124 (FIG. 2B). The production of miR-124 also correlated with viral replication as measured by PB2 synthesis (FIG. 2C). To ensure that the production of miR-124 from influenza A virus was processed by the endogenous cell machinery, infections with the scrambled control and miR-124-producing viruses in wild type and Dicer deficient fibroblasts were performed. Total RNA was analyzed by small RNA Northern blot, demonstrating miR-124 production exclusively in wild-type cells infected with the miR-124-encoding influenza A virus (FIG. 2D). Loss of miRNA production, as a result of Dicer deficiency, was confirmed by an absence of miR-93 expression. These results were further corroborated through stem-loop specific RT-PCR (FIG. 2E). Taken together, these results demonstrate that influenza A virus can be engineered to deliver high levels of miR-124 in the context of a de novo virus infection.

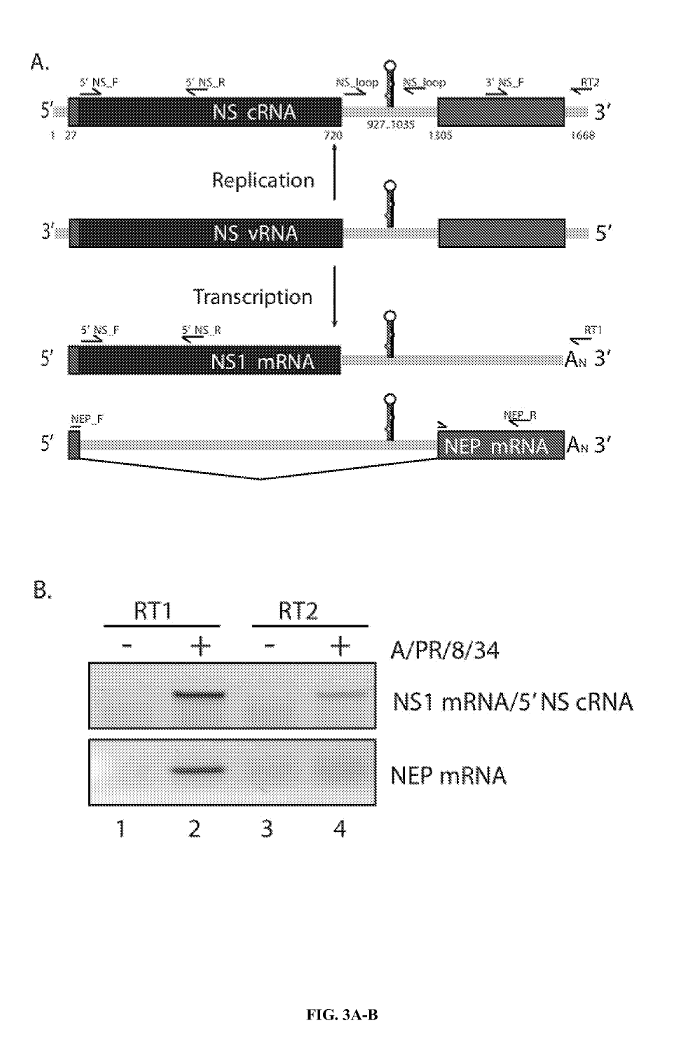

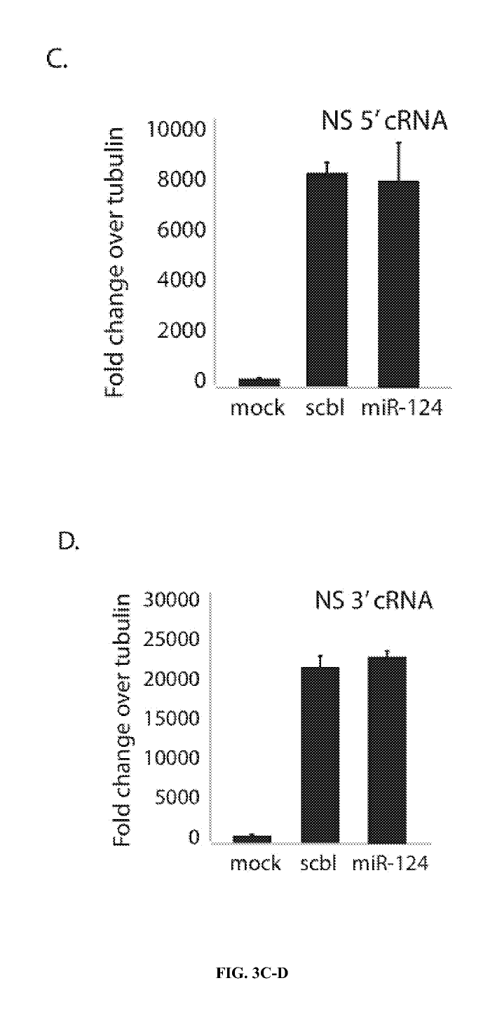

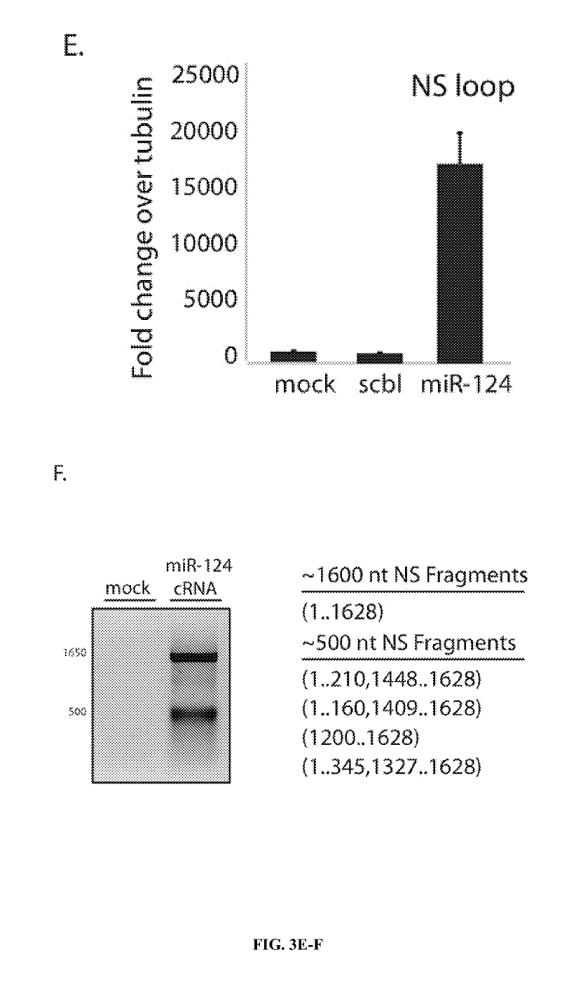

One of the RNA viral constraints of encoding a miRNA is the hairpin itself could form a Drosha substrate during viral replication that would result in genomic splicing, producing two distinct fragments and the miRNA hairpin. This phenomenon would clearly impact viral progeny output and possibly induce the formation of defective interfering (DI) particles. In the case of influenza A virus, cleavage of the miR-124 hairpin could result in fragmentation of viral cRNA at the base of the miR-124 stem (FIG. 3A). To monitor cRNA levels for cleavage activity, reverse transcription (RT) on RNA from fibroblasts infected with scrambled control or miR-124-containing viruses utilizing an oligo dT primer or a primer specific for the 3′ cRNA non-coding region (NCR), which is absent in both NS1 and NEP/NS2 mRNA (see, e.g., Palese and Shaw (2007) in Fields Virology 5th Edition, eds Knipe, D M and Howley, P M. (Raven, Philadelphia), pp 1648-1698), was performed. Whereas oligo dT RT synthesized both NS1 and NEP/NS2 mRNA (as well as NS cRNA), 3′ cRNA RT selectively amplified NS cRNA and excluded mRNA as evident by the lack of NEP/NS2 (FIG. 3B). To determine whether Drosha was capable of processing the miRNA hairpin directly from the genome, this discriminating RT reaction was used to monitor the 5′, 3′, and hairpin region of the cRNA during de novo virus infection. Quantitative PCR (qPCR) of the NS segment demonstrated that the 5′ and 3′ ends were equally represented between the scrambled control and the miR-124-producing influenza A viruses (FIGS. 3C and 3D). Equal representation of the 5′ and 3′ segment ends suggests that the level of NS synthesis between these two viruses was comparable. To ensure that the qPCR data did not reflect the emergence of a viral revertant, primers specific for the miR-124 NS loop were used to demonstrate that the genomic hairpin was still present (FIG. 3E). As cleavage of cRNA would result in the inhibition of further vRNA/cRNA synthesis, the comparable levels of cRNA strongly suggest that viral genomic RNA is not a favorable substrate for Drosha-mediated cleavage. To determine whether genomic RNA was processed by Drosha at any level, 5′ RACE was performed (rapid amplification of 5′ complementary ends) on cRNA (FIG. 3F). In addition to the full length cRNA product, this analysis amplified a second aberrant cRNA species from the miR-124-producing virus. Upon sequencing, this ˜500 nucleotide product was identified as a heterogenous population of cRNAs. While some species isolated included 5′ and 3′ cRNA ends with large internal deletions, none of the fragments terminated at the base of the miR-124 hairpin; suggesting random replication intermediates rather than Drosha-mediated activity. In all, lack of Drosha activity on either NS cRNA (FIG. 3) or the 3′ UTR of NS1 (FIG. 6) demonstrates that the sole source of miR-124 is the lariat produced during NEP/NS2 synthesis.

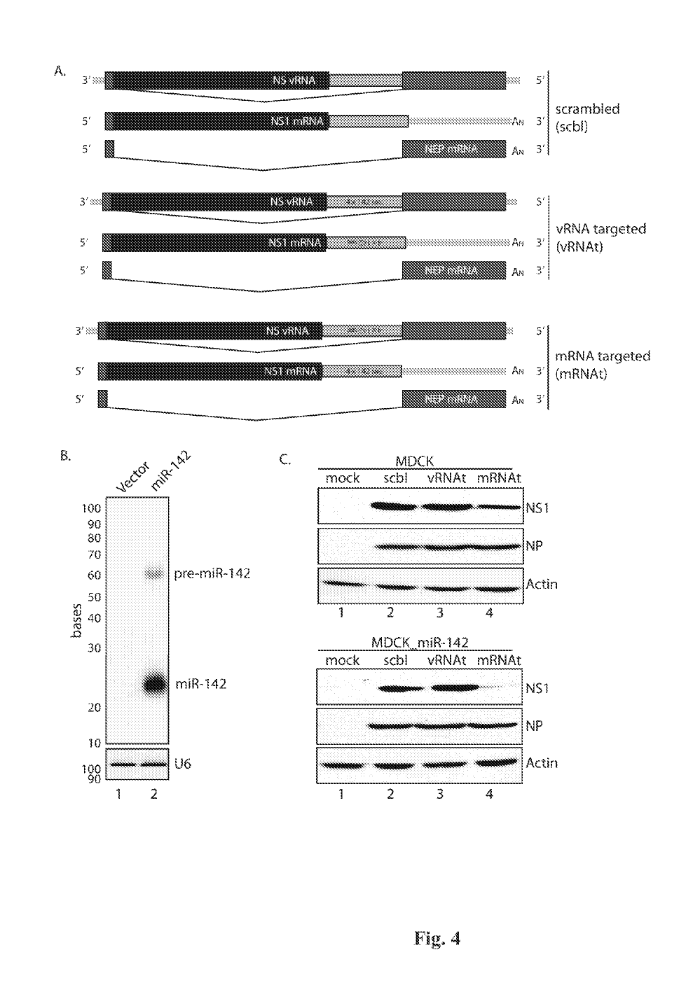

A second hindrance of encoding a miRNA in the context of an RNA viral genome, is that the genomic strand that encodes the intronic hairpin becomes a perfect inverse complement to the produced miRNA, therefore serving as a potential miRNA target. In the context of influenza, a hairpin produced from mRNA would result in the formation of a miRNA target on the vRNA. This would not occur in the context of cRNA or mRNA because of the imperfect binding along miRNA stem loops. To determine whether this phenomenon causes a significant restriction on RNA virus produced miRNAs, additional viruses were engineered to determine whether the vRNA could be subject to miRNA-mediated inhibition. For these studies, the segment eight encoding an intergenic region was used to introduce miR-142 target sites in either the 3′ UTR of NS1 or in the context of vRNA (FIG. 4A). Exogenous expression of miR-142 was achieved by plasmid delivery of the miR-142 hairpin and confirmed by small RNA Northern blot (FIG. 4B). As this miRNA has already been demonstrated to potently induce transcriptional inhibition of miR-142 targets (see, e.g., Brown et al. (2007) Nat Biotechnol 25(12):1457-1467), it was investigated whether the levels of NS1 would be affected when the mRNA (mRNAt) and/or vRNA (vRNAt) was targeted. MDCK cells, or MDCK cells stably expressing miR-142, were infected with a scrambled control, mRNAt or vRNAt recombinant viruses at an MOI of 0.1 for 18 hours (FIG. 4C). Total protein analysis demonstrated that NS1 levels in control (ctrl) and vRNAt recombinant viruses showed no significant difference regardless of miR-142 expression. In contrast, miR-142 targeting of mRNA (mRNAt), resulted in a dramatic loss of NS1 in a miR-142 dependent manner, while viral NP levels remain unaffected. Altogether, these results suggest that the accessibility of genomic RNA to the miRNA/RISC complex is not sufficient to affect the overall transcript levels of the virus.

Finally, to assess if virus-produced miRNAs are loaded into the RISC complex and capable of mediating PTGS, it was determined whether a green fluorescent protein (GFP) encoding tandem repeats of miR-124 target elements (GFP_{—}124) could be silenced. Recombinant viral infections and subsequent GFP_{—124} transfections demonstrated a 47.4% decrease in the number of green fluorescent cells only in the context of the miR-124 expressing influenza A virus (FIG. 5A). Furthermore, to ensure that virus infection could induce PTGS on an endogenous cellular transcript, a neuronal precursor cell line (CAD) was used to determine whether miR-124 expression could stimulate neuron-like differentiation as previously described (see, e.g., Makeyev et al. (2007) Mol Cell 27(3):435-448). To this end, CAD cells were untreated, serum-starved, or infected with the scrambled or miR-124 producing influenza A virus strains (FIG. 5B). At 24 hours post-infection, or 48 hours post-serum starvation, cells were fixed and examined by confocal microscopy demonstrating that serum starvation, or expression of virus-produced miR-124, was sufficient to induce neuron-like morphology. Taken together, these results demonstrate that influenza A virus can be engineered to encode an endogenous, fully functional, miRNA.

6.1.3 Conclusion

An influenza A virus strain was engineered to encode a functional miRNA which was synthesized to levels comparable to highly abundant cellular miRNAs. The virus-generated miRNAs mimicked their endogenous counterparts in their ability to confer PTGS on target mRNAs.

### 6.2 Example 2

This example demonstrates that Sindbis virus, a positive single stranded cytoplasmic virus, can be engineered to produce functional miRNA.

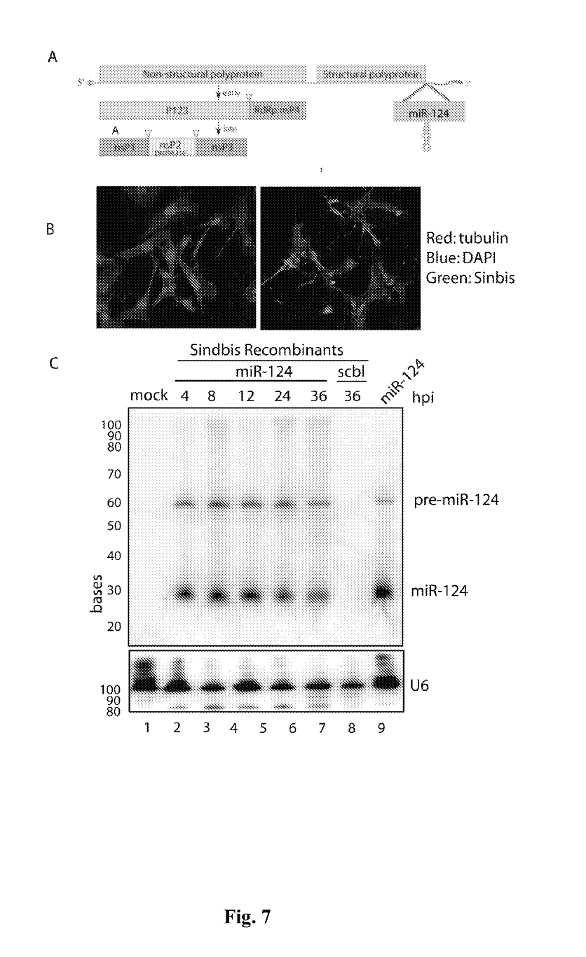

The mmu-pri-miR-124-2 locus (chr3:17,695,454-17,696,037) was inserted into a a unique BstEII restriction site downstream of the structural genes of Sindbis virus (Strain s51) and included a duplicate subgenomic promoter (FIG. 7A). The recombinant strain (Sindbis-124) is able to infect CAD cells (FIG. 7B) and produces both pre-miR-124 and miR-124 in human fibroblasts from 4 through 36 hours post infection at an MOI of 1.0 (FIG. 7C).

6.2.1 Sindbis-Produced miR-124 Requires Dicer but is Exportin-5 Independent

Exportin-5-positive 293 fibroblasts, exportin-5-negative 293 fibroblasts, dicer-positive immortalized murine fibroblasts, and dicer-negative immortalized murine fibroblasts were infected with a mock control, Sindbis-124, or Sindbis virus (Strain s51) encoding a scrambled (scbl) RNA locus, and the ability of the cells to process pre-miR-124 and produce miR-124 was assessed. Exportin-5-positive and exportin-5-negative cells infected with Sindbis-124 produced miR-124 (FIG. 13, lanes 9 and 12). In contrast, only dicer-positive cells infected with Sindbis-124, and not dicer-negative cells infected with Sindbis-124, produced miR-124 (FIG. 13, lanes 3 and 6). Thus, Sindbis-produced miR-124 requires Dicer for processing but does not require exportin-5 for processing, indicating that production of miR-124 by Sindbis virus is nucleus-independent (FIG. 13).

### 6.3 Example 3

MicroRNA can be generated that targets a gene of interest using model miRNA. To generate such artificial miRNA that targets a gene of interest from model RNA, certain parameters can be followed, such as (i) the overall predicted structure of the model miRNA can be conserved in the artificial miRNA; (ii) the artificial miRNA can contain the 5′ and 3′ flanking sequences of the model pre-miRNA; (iii) the buldge of the hairpin can be identical between the artificial and model miRNAs; and (iv) the complementarity along the stem of the artificial miRNA can match that of the model miRNA.

6.3.1 Human NFKBIA Gene

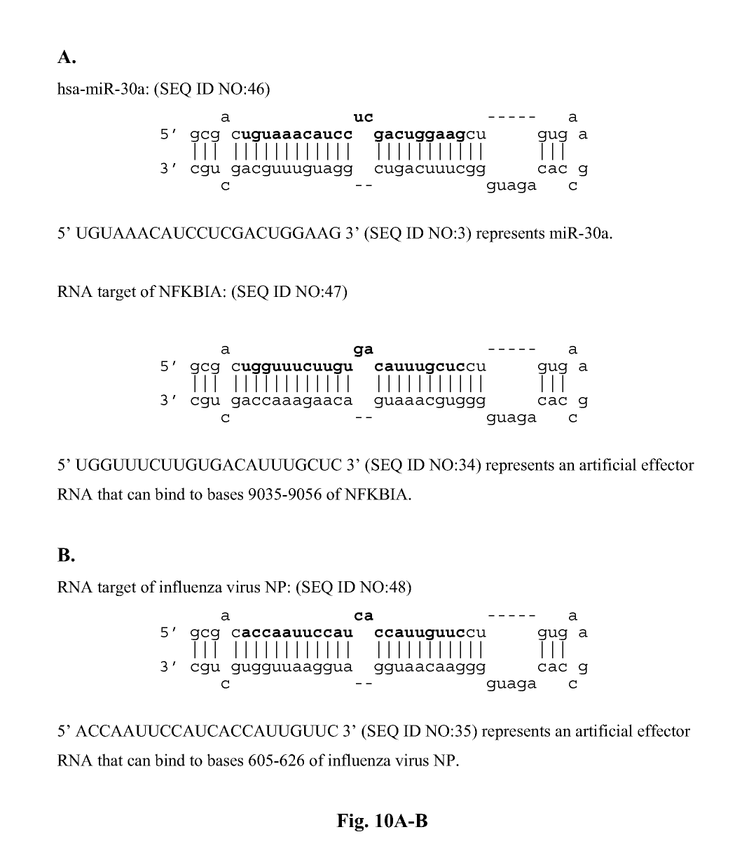

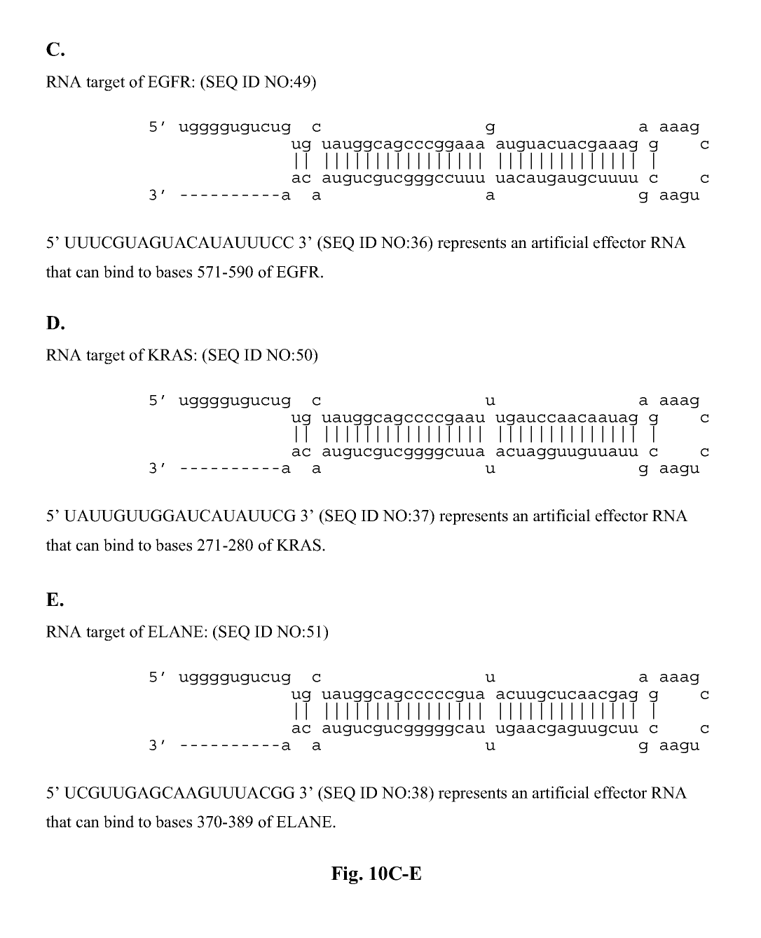

To target the human NFKBIA gene (accession number NG 007571.1; GENE ID NO: 4792), a heterologous RNA can be designed, modeled after miR-30a (GENE ID NO: 407029), as shown in FIG. 10A.

6.3.2 Influenza Virus Nucleoprotein Gene

To target an influenza virus nucleoprotein gene (accession number EF190975.1), a heterologous RNA can be designed, modeled after miR-30a, as shown in FIG. 10B.

6.3.3 Human EGFR Gene

To target a human EGFR gene (Gene ID: 1956), a heterologous RNA can be designed, modeled after has-miR-585 (gene ID 693170), as shown in FIG. 10C.

6.3.4 Human KRAS Gene

To target a human KRAS gene (Gene ID: 3845), a heterologous RNA can be designed, modeled after has-miR-585 (gene ID 693170), as shown in FIG. 10D.

6.3.5 Human ELANE Gene

To target a human ELANE gene (Gene ID: 1991), a heterologous RNA can be designed, modeled after has-miR-585 (gene ID 693170), as shown in FIG. 10E.

6.3.6 Shigella HepA Gene

To target a Shigella Hep A gene (Accession Number NC_{—}008258.1), a heterologous RNA can be designed, modeled after has-miR-585 (gene ID 693170), as shown in FIG. 10F.

6.3.7 SARS Coronavirus Nucleoprotein Gene

To target a SARS coronavirus nucleoprotein gene (Accession Number AY291315.1), a heterologous RNA can be designed, modeled after has-miR-585 (gene ID 693170), as shown in FIG. 10G.

### 6.4 Example 4

Recombinant RNA viruses comprising an effector RNA that targets a gene of interest can be generated.

6.4.1 Segmented, Negative-Stranded RNA Viruses

Recombinant segmented, negative-stranded RNA viruses (e.g., orthomyxoviruses) can be generated that produce effector RNA.

6.4.1.1 Lariat—Classical

Recombinant segmented, negative-stranded RNA viruses (e.g., orthomyxoviruses) can be generated that comprise a gene segment that comprises an effector RNA that forms a classical lariat. The recombinant segmented, negative-stranded RNA virus can comprise a gene segment that comprises: (a) packaging signals found in the 3′ non-coding region of a gene segment of the recombinant segmented, negative-stranded RNA virus; (b) a first nucleotide sequence that forms part of an open reading frame of a gene of the recombinant segmented, negative-stranded RNA virus; (c) a splice donor site; (d) a heterologous RNA sequence; (e) a splice acceptor site; (f) a second nucleotide sequence that forms part of the open reading frame of the gene of the recombinant segmented, negative-stranded RNA virus; and/or (g) packaging signals found in the 5′ non-coding region of a gene segment of the recombinant segmented, negative-stranded RNA virus.

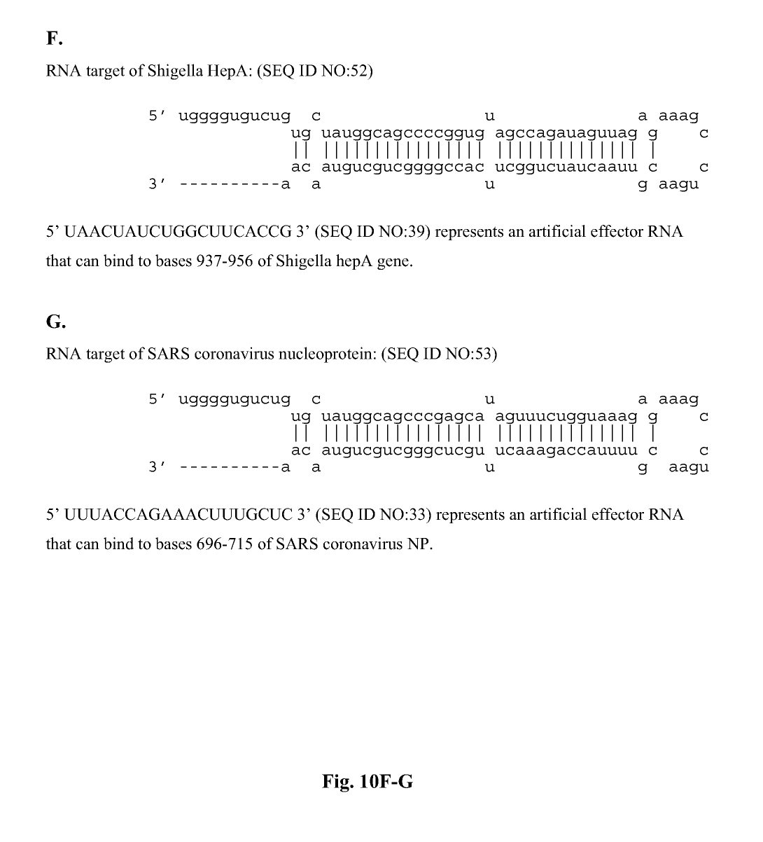

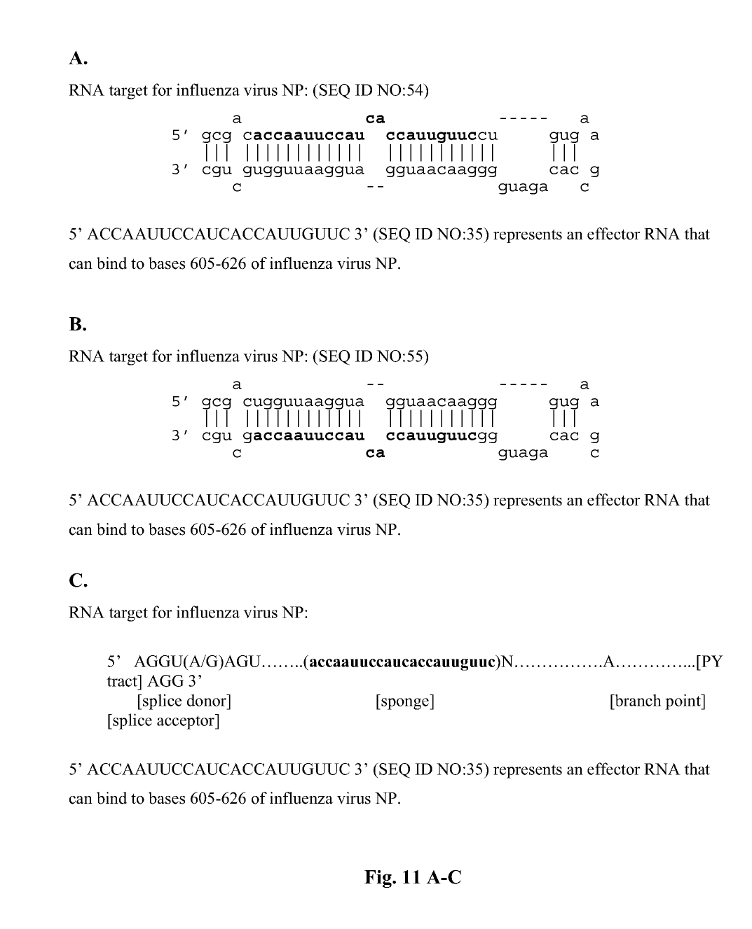

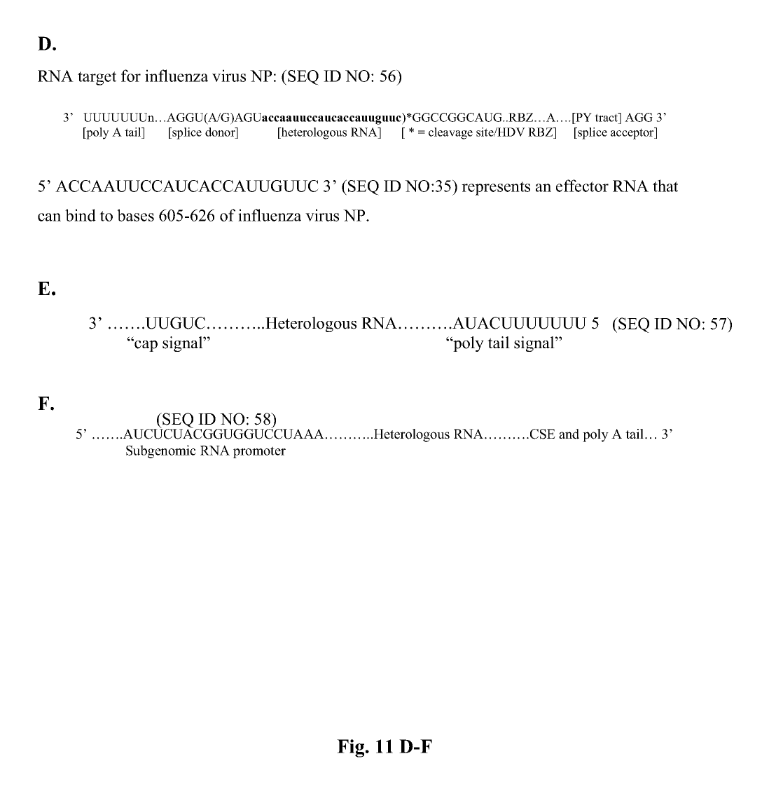

To target an influenza virus nucleoprotein gene (accession number EF190975.1), a heterologous RNA can be designed that would function as a classical lariat, as shown in FIG. 11A.

6.4.1.2 Lariat—Cytoplasmic Passenger Strand Delivery

Recombinant segmented, negative-stranded RNA viruses (e.g., orthomyxoviruses) can be generated that comprise a gene segment that comprises an effector RNA that forms a lariat for cytoplasmic passenger strand delivery. The recombinant segmented, negative-stranded RNA virus can comprise a gene segment that comprises: (a) packaging signals found in the 3′ non-coding region of a gene segment of the recombinant segmented, negative-stranded RNA virus; (b) a first nucleotide sequence that forms part of an open reading frame of a gene of the recombinant segmented, negative-stranded RNA virus; (c) a splice donor site; (d) a heterologous RNA sequence designed to form a hairpin with the sequence of interest being excluded from RISC; (e) a splice acceptor site; (f) a second nucleotide sequence that forms part of the open reading frame of the gene of the recombinant segmented, negative-stranded RNA virus; and/or (g) packaging signals found in the 5′ non-coding region of a gene segment of the recombinant segmented, negative-stranded RNA virus.

To target an influenza virus nucleoprotein gene (accession number EF190975.1), a heterologous RNA can be designed that would function as a lariat for cytoplasmic passenger strand delivery, as shown in FIG. 11B.

6.4.1.3 Lariat—Nuclear Sponge

Recombinant segmented, negative-stranded RNA viruses (e.g., orthomyxoviruses) can be generated that comprise a gene segment that comprises an effector RNA that forms a lariat that acts as a nuclear sponge. The recombinant segmented, negative-stranded RNA virus can comprise a gene segment that comprises: (a) packaging signals found in the 3′ non-coding region of a gene segment of the recombinant segmented, negative-stranded RNA virus; (b) a first nucleotide sequence that forms part of an open reading frame of a gene of the recombinant segmented, negative-stranded RNA virus; (c) a splice donor site; (d) an intron encoding tandem repeats of complementary RNA to a desired RNA target; (e) a splice acceptor site; (f) a second nucleotide sequence that forms part of the open reading frame of the gene of the recombinant segmented, negative-stranded RNA virus; and/or (g) packaging signals found in the 5′ non-coding region of a gene segment of the recombinant segmented, negative-stranded RNA virus.

To target an influenza virus nucleoprotein gene (accession number EF190975.1), a heterologous RNA can be designed that would function as a lariat that acts as a nuclear sponge, as shown in FIG. 11C.

6.4.1.4 Ribozyme Liberated

Recombinant segmented, negative-stranded RNA viruses (e.g., orthomyxoviruses) can be generated that comprise a gene segment that comprises an effector RNA that is liberated by a ribozyme. The genome of the recombinant segmented, negative-stranded RNA virus can comprise: (a) packaging signals found in the 3′ non-coding region of a gene segment of the recombinant segmented, negative-stranded RNA virus; (b) a first nucleotide sequence that forms the open reading frame of a gene of the recombinant segmented, negative-stranded RNA virus; (c) a stretch of greater than ten uracil bases; (d) a splice donor site; (e) a heterologous RNA sequence; (e) a ribozyme recognition motif; (f) a self-catalytic RNA (e.g. Hepatitis delta ribozyme); (g) a splice acceptor site; and/or (h) packaging signals found in the 5′ non-coding region of a gene segment of the recombinant segmented, negative-stranded RNA virus.

To target an influenza virus nucleoprotein gene (accession number EF190975.1), a heterologous RNA can be designed that comprises an effector RNA that is liberated by a ribozyme as shown in FIG. 11D.

6.4.2 Single-Stranded, Negative Sense RNA Viruses

Recombinant single-stranded, negative sense RNA viruses (e.g., viruses from the family rhabdoviridae or paramyxoviridae) can be generated that produce effector RNA. The genome of the recombinant single-stranded, negative sense RNA viruses can comprise: (a) polymerase initiation sites found in the 3′ non-coding region of the genome of the recombinant single-stranded, negative sense RNA virus; (b) any number of viral segments required for viral replication of the recombinant single-stranded, negative sense RNA virus; (c) a heterlogous RNA sequence whose 5′ and 3′ sequences adhere to the requirements for polymerase initiation and termination; (d) any remaining viral segments required for viral replication; and/or (e) polymerase replication sites found in the 5′ non-coding region of the genome of the recombinant single-stranded, negative sense RNA virus.

To target a gene from a virus of the family rhabdoviridae a genomic region can be designed as shown in FIG. 11E.

6.4.3 Single-Stranded, Positive Sense RNA Viruses

Recombinant single-stranded, positive sense RNA viruses (e.g., viruses from the family togaviridae) can be generated that produce effector RNA. The genome of the recombinant single-stranded, positive sense RNA viruses can comprise: (a) polymerase initiation sites found in the 5′ non-coding region of the genome of the recombinant single-stranded, negative sense RNA virus; (b) the open reading frame for the non-structural viral proteins; (c) the internal recognition sequence for subgenomic RNA synthesis; (d) the open reading frame for the structural viral proteins; (d) a second internal recognition sequence for subgenomic RNA synthesis; (e) a heterlogous RNA sequence whose 5′ and 3′ sequences adhere to the requirements for polymerase initiation and termination; and/or (f) polymerase replication sites found in the 3′ non-coding region of the genome of the recombinant single-stranded, negative sense RNA virus including the 3′ conserved sequence element (CSE) and the poly A tail.

To target a gene from a virus of the family togaviridae a genomic region can be designed as shown in FIG. 11F.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### 6.5 Example 5

This example demonstrates that Sindbis virus-derived miR-124 is a DGCR8-independent, functional microRNA and that Sindbis-derived miR-124 can be generated by Dicer and utilized in an antiviral capacity in vertebrate cells.

6.5.1 Sindbis-Derived miR-124 is a DGCR8-Independent, Functional MicroRNA

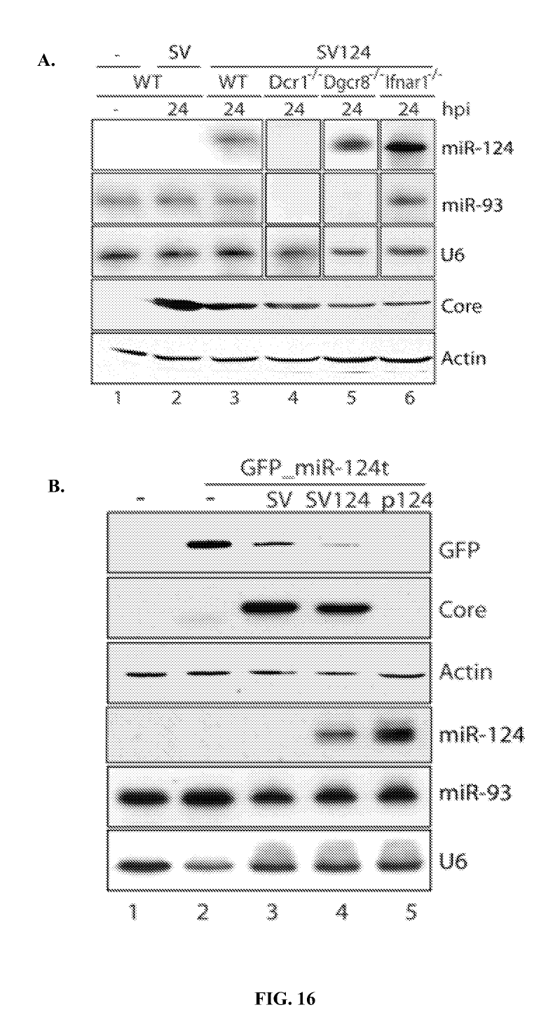

Sindbis virus that produces miR-124 (Sindbis-124) was engineered as described in Example 2. To explore the molecular nature of Sindbis-derived miR-124, wild-type murine fibroblasts were infected with the engineered virus and the RNA from these infections was compared to that from identical experiments performed using fibroblasts lacking either Dicer, DGCR8, or the IFN-I receptor component IFNAR1 (FIG. 16A). Following 24 hours of infection, wild type murine fibroblasts demonstrated robust synthesis of miR-124 specifically from Sindbis-124 infections. As demonstrated in Example 2, Sindbis-generated miR-124 is dependent upon Dicer activity, however, synthesis of Sindbis-derived miR-124 was not dependent on DGCR8, the essential RNA-binding component of the microprocessor. This is in contrast to endogenous miR-93, which, like cells lacking Dicer, deletion of DGCR8 results in a complete loss of the endogenous miRNA. It also was determined whether the DGCR8- and Exportin-5-independent generation of Sindbis-derived miR-124 required an antiviral-specific component. To do so, cells lacking a functional IFN-I receptor were infected with Sindbis-124. These cells, similar to those with loss of DGCR8 or Exportin-5, demonstrate robust miR-124 synthesis with no evidence of cross-talk between the observed non-canonical processing and the cell's autonomous antiviral defenses.

To determine whether Sindbis-derived miR-124 was functional, an artificial construct was constructed in which green fluorescent protein (GFP) included a 3′ UTR with tandem repeats of the reverse complement of miR-124 (GFP_miR-124t), thereby making it susceptible to PTGS activity (FIG. 16B). Transfection of GFP_miR-124t resulted in robust GFP expression in the absence of any other treatment. In contrast, p124 induced PTGS of GFP_miR-124t to a level below Western blot detection. Furthermore, whereas treatment with Sindbis virus resulted in a general decrease in host protein synthesis, a common attribute amongst alphaviruses, this effect was significantly enhanced by the production of Sindbis-derived miR-124, suggesting virus-produced miR-124 was capable of inducing PTGS.

Thus, Sindbis-derived miR-124 is a DGCR8-independent, functional microRNA, which is in contrast to Influenza A virus produced miR-124, which depends on DGCR8, a result of the fact that Influenza A viruses are nuclear (see, e.g., Varble et al., 2011, RNA Biology 8:190-194).

6.5.2 Sindbis-Derived miR-124 can be Utilized in an Antiviral Capacity

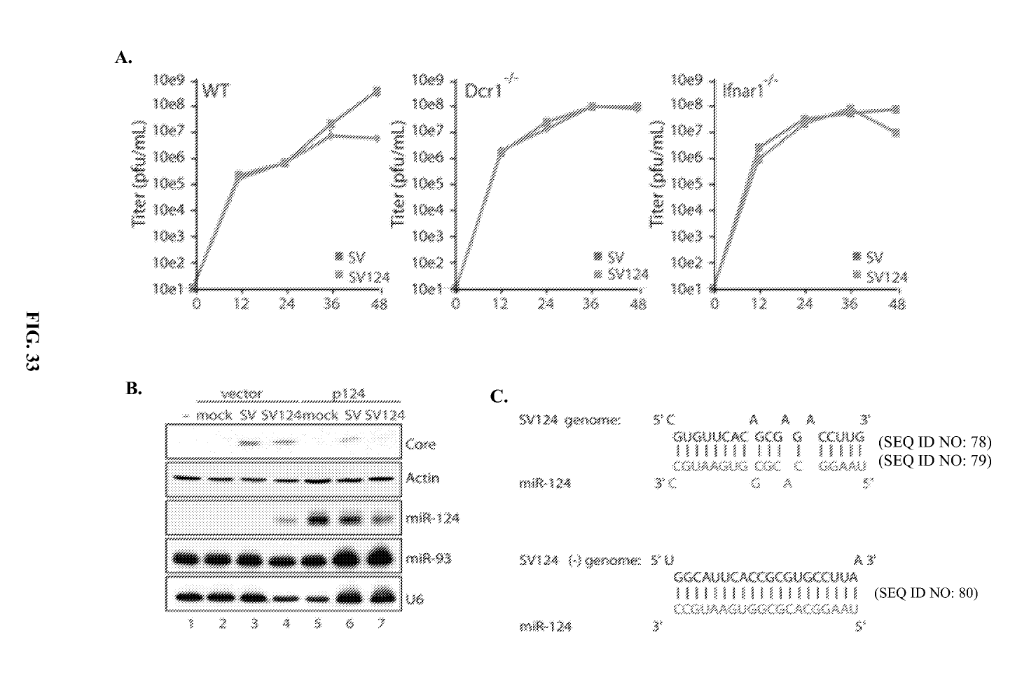

As processing of Dgcr8-independent, Dicer-dependent small RNAs produced by Sindbis virus in many ways mimics the antiviral response in invertebrates, whether Sindbis-derived miR-124 could be generated by Dicer and utilized in an antiviral capacity in vertebrate cells was investigated. While no evidence of attenuation in immortalized human fibroblasts was observed, rapid replication in these cells in response to infections performed at high MOIs may have masked this phenotype. Therefore, the viral replication properties of SV and Sindbis-124 in wild type (WT), Dcr1^{-/-} and Ifnar1^{-/-} fibroblasts at a low MOI (FIG. 33A) were compared. While both SV and Sindbis-124 infections amplified to high titers in WT cells by 48 hours post-infection, Sindbis-124 demonstrated a ˜2 log attenuation (p=0.008). This attenuation was not the result of steric hindrance on the RdRp or increased PAMP production as the levels between SV and Sindbis-124 were not significantly different in Dicer knockout cells (p=0.164) but still maintained a 1 log difference in the absence of IFN-I signaling (p=0.015).

To demonstrate that miR-124 could be used to target virus directly, fibroblasts were transfected with vector alone or p124 and subsequently mock treated or infected these cells with SV or Sindbis-124 (FIG. 33B). While expression of plasmid-derived miR-124 had no impact on SV core levels, Sindbis-124 protein was reduced by 5.8 fold. miR-124 targeting of Sindbis-124 likely occurs at the level of the negative strand (−) genome as the mean free energy (mfe) of the miR-124 target on the genome is only −24.9 kcal/mol and does not contain a seed sequence greater than 6-nts (FIG. 33C). This is in contrast to the (−) genome which has a perfect miR-124 target and an mfe of −45.1 kcal/mol.

### 6.6 Example 6

This example demonstrates that an artificial microRNA is as effective as traditional siRNA at disrupting specific gene expression based on the fact that the artificial microRNA-producing vectors can generate comparable levels of miRNA as compared to standard siRNA transfections.

As demonstrated in Example 3, microRNA can be generated that targets a gene of interest using model miRNA by following certain parameters. As another example, to target the human STAT1 gene (Gene ID: 6772; Accession Number GU211348.1), a heterologous RNA can be designed, modeled after miR-124, as shown in FIG. 17A-B. The mature amiRNA depicted in FIG. 17A (SEQ ID NO:45) binds to positions 478 to 497 of human STAT1.

To determine whether the hairpin engineered to produce STAT1 siRNA (termed STAT1 amiRNA) could be expressed at the cellular level, human lung alveolar cells (A549) were mock-transfected or transfected with either STAT1 siRNA or STAT1 amiRNA, followed by Northern blot analysis with probing for STAT1 siRNA (or U6 RNA as a control). As shown in FIG. 17C, expression of both STAT1 siRNA or STAT1 amiRNA was detected, indicating expression of the STAT1 siRNA in each instance.

Next, to determine whether the artificial STAT1 siRNA (STAT1 amiRNA) was efficient at disrupting STAT1 gene expression, human lung alveolar cells (A549) were transformed with a plasmid expressing STAT 1 amiRNA or a plasmid expressing wild-type miR-24 and cultured in the presence and absence of universal interferon beta (PBL Biomedical) at a concentration of 100 units/mL for 12 hours. Subsequently, Western blot analysis was performed with probing for STAT1 protein expression (or beta-actin as a control). As demonstrated in FIG. 17D, the artificial STAT1 siRNA (STAT1 amiRNA) efficiently knocked down STAT1 gene expression as indicated by the absence of STAT1 protein expression both in the presence and absence of IFN-I.

### 6.7 Example 7

This example demonstrates in vivo evidence for nuclear-independent synthesis of miRNAs in viruses and validates the molecular components of this nuclear-independent pathway as they compare to canonical miRNA synthesis.

6.7.1 Materials and Methods

### 6.7.1.1 Small RNA Northern Blot Analyses and Deep Sequencing

Small RNA Northern blots and probe labeling were performed as previously described (see Perez et al., Proc Natl Acad Sci USA 107, 11525-11530 (2010); and Pall and Hamilton, Nat Protoc 3, 1077-1084 (2008)). Probes used included: anti-miR-124: 5′-TGGCATTCACCGCGTGCCTTAA-3′ (SEQ ID NO:40), anti-miR-93: 5′-CTACCTGCACGAACAGCACTTTG-3′ (SEQ ID NO:41), and anti-U6: 5′-GCCATGCTAATCTTCTCTGTATC-3′ (SEQ ID NO:42), anti-miR-122: 5′-CAAACACCATTGTCACACTCCA-3′(SEQ ID NO:43) and anti-miR-124-star: 5′-ATCAAGGTCCGCTGTGAACACG-3′ (SEQ ID NO:44). For deep sequencing analysis, miR-124-specific small RNA libraries were generated as previously described (see Pfeffer et al., Nat Methods 2, 269-276 (2005)). Total RNA from Sindbis virus (SV), VSV and Influenza A virus (IAV) expressing miR-124 infected samples was extracted 16 hours post infection and small RNA species were separated on a 12% denaturing tris-urea gel. Small RNA species were then isolated, purified and amplified as previously described (see Shapiro et al., RNA 16, 2068-2074 (2010)). Samples were then run on a Illumina GA llx hiseq 2000 sequencing machine and mapped to the pri-miR-124-2 locus.

6.7.1.2 Vector Design for Cytoplasmic miR-124 Synthesis

Generation of Sindbis and Influenza A viruses expressing miR-124 have been described elsewhere (see Varble, et al., Proc Natl Acad Sci USA 107, 11519-11524 (2010); Shapiro et al., RNA 16, 2068-2074 (2010)). VSV expressing the pri-miR-124 genomic segment (chr3:17,695,454-17,696,037) was generated and rescued as previously described (see Stojdl et al., Cancer Cell 4, 263-275 (2003)). Similarly, SV122 was generated by cloning pri-miR-122 (genomic coordinates) into an artificial subgenomic promoter as previously described (see Shapiro et al., RNA 16, 2068-2074 (2010)). GFPmiR-124 was generated from the plasmid pEGFP-C1 (GenBank Accession# U55763). The pri-miR-124 3′ untranslated region was generated using the mmu-miR-124-2 murine locus (chr3:17,695,454-17,696,037) which was ligated into pCR TOPO 2.1 (Invitrogen) and subcloned using XhoI and BamHI. The pCR TOPO 2.1 clone of pri-miR-124 was PCR amplified with T7 and M13R primers and transfected as a PCR fragment with pCAGGs T7 polymerase.

6.7.1.3 Cell Culture

Dicer1^{-/-} and Argonaute2^{-/-} fibroblasts were obtained from Alexander Tarakhovsky (Rockefeller University) and Donal O'Carroll (EMBL, Monterotondo, Italy) (see Perez et al., Nat Biotechnol 27, 572-576 (2009); and O'Carroll et al., Genes Dev 21, 1999-2004 (2007)). RNasen^{fl/fl} fibroblasts were obtained from Dan Littman (NYU) (see Chong et al., Genes Dev 24, 1951-1960 (2010) and were cultured in media supplemented with pyruvate. Dgcr8^{fl/fl} fibroblasts were obtained from Robert Blelloch (UCSF). TRBP2^{-/-} fibroblasts were obtained from Anne Gatignol, (McGill University) as described (see Zhong et al., Nat Genet 22, 171-174 (1999)). PACT^{-/-} fibroblasts were obtained from Ganes C. Sen (Cleveland Clinic) (see Patel et al., EMBO J 17, 4379-4390 (1998)). All cells were cultured in DMEM supplemented with 10% FBS and penicillin/streptomycin unless otherwise indicated. Floxed cells were infected with Adenovirus expressing GFP or GFP_Cre (vector biolabs #1060 and #1700, respectively) at an MOIs of 300 and 500 and subsequently treated as described 5 days post-Adenovirus infection. Serum starvation experiments were performed by washing the cells and incubating them with serum-free media. To confirm loss of cell division, cells were incubated with 10 um CFSE (molecular probes) for 10 mins at 37° C. CFSE was quenched with 25% BSA, washed and replated in either DMEM with or without 10% serum. At 24 and 48 hours post CFSE labeling cells were fixed (BD FACS lysis solution), run on a FACS Calibur (BD) and analysed using Flojo (Treestar). Post transcriptional silencing of mIR-124, as measured by luciferase, was performed in baby hamster kidney (BHK) cells as previously described (see Perez et al., Nat Biotechnol 27, 572-576 (2009)). Briefly, cells were transfected with luciferase containing scp1 within the 3′ UTR and infected with either WT or miR-124-expressing SV, VSV and IAV at MOI's of 3, 0.5 and 5 respectively. Twelve hours post-infection, cells were lysed and analyzed as per the manusfacturer's instruction. All luciferase values were normalized to renilla and were performed in triplicate. Luciferase expression from 124 expressing virus was compared to WT virus infection. For post-transcriptional silencing of GFP, BHK cells were transfected with miR-124-targeted GFP (GFP 124t-3′UTR) as previously described (see Varble, et al., Proc Natl Acad Sci USA 107, 11519-11524 (2010)) and either co-transfected with a plasmid expressing miR-124 (p124) or infected with SV124, VSV124 and IAV124 at MOI of 1, 3, 5 respectively 2 hours post-transfection. Sixteen hours post infection, protein was extracted and analyzed by Western blot.

6.7.1.4 In Vivo Infections

IFNαR1^{-/-} mice were anesthetized with isofluorane and infected i.v. with 2×10⁵ pfu SV124 or i.n. with 2×10⁷ pfu of VSV 124 or 1×10⁷ pfu of IAV 124. Lungs were removed on day 1 p.i. for VSV and day 2 p.i. for SV and IAV.

6.7.1.5 Western Blot Analysis, Immunoprecipations, and qPCR

Western blot analysis was performed as previously described (see Perez et al., Proc Natl Acad Sci USA 107, 11525-11530 (2010)). Antibodies used included: anti-Pan-actin (NeoMarkers, Freemont Calif.), anti-GFP polyclonal (Santa Cruz Biotechnologies sc-73556sc), anti-SV core (ATCC, VR-1248AF), anti-VSV G (Genscript—A00199), anti-Flag (sigma) and anti-IAV NP (BEI Resources). Blots were incubated with secondary rabbit or mouse antibodies at a 1:5000 for 1 hour at room temperature. Immunobilon Western Chemiluminescent HRP Substraight (Millipore) was used as per manufacturer's instructions. Immunoprecipations were perfermed in 293 cells. Cells were transfected with 12 μg of either flag-tagged Ago2 or flag-tagged GFP and subsequently infected with either wild-type or miR-124 expressing SV, VSV or IAV. Protein extracts were harvested 12 hours post-infection and were immunoprecipitated with Protein-G-PLUS agarose (Santa Cruz Biotechnologies) and 10 μg of anti-Flag (Sigma) for 12 hours at 4° C. Beads were washed and RNA extracted with TRIzol (Invitrogen). qPCR of cDNA samples was performed using KAPA SYBR FAST qPRC Master Mix (KAPA Biosystems). PCR reactions were performed on a Mastercycler ep realplex (Eppendorf). Actin was used as the endogenous housekeeping gene and Delta delta cycle threshold (AACT) values were calculated with replicates over actin. Values represent the fold change over mock-infected samples.

6.7.1.6 Statistical Analysis

Statistical analysis was performed on indicated samples using a two tailed, unpaired student's T test. Data are considered significant if the p value is less than 0.05.

6.7.2 Results

6.7.2.1 Cytoplasmic-Mediated Synthesis of miRNAs

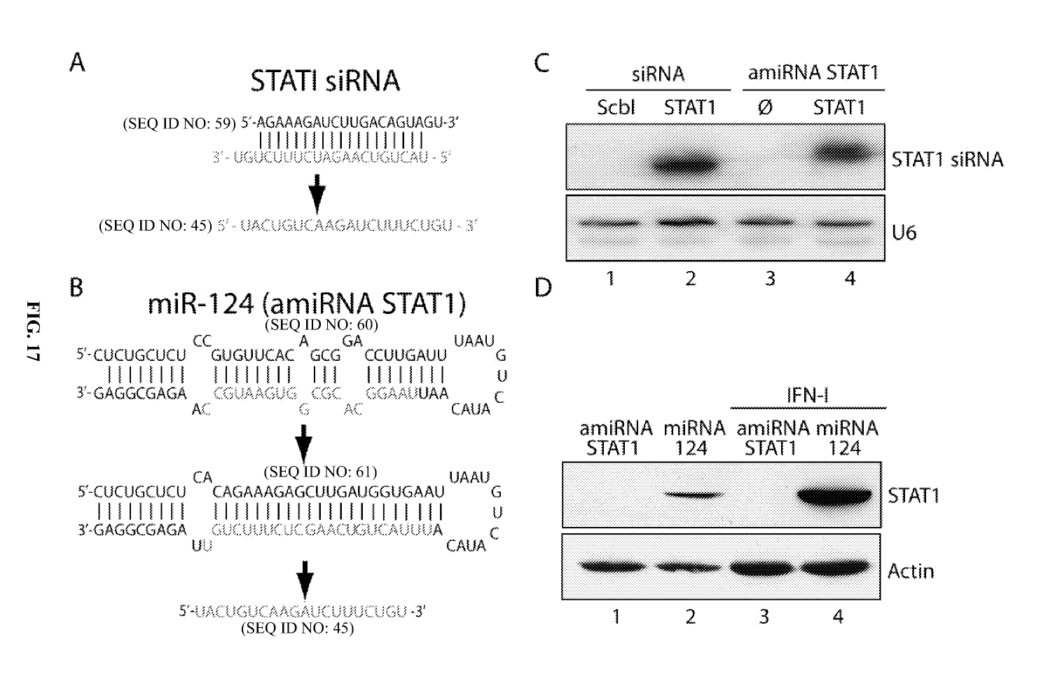

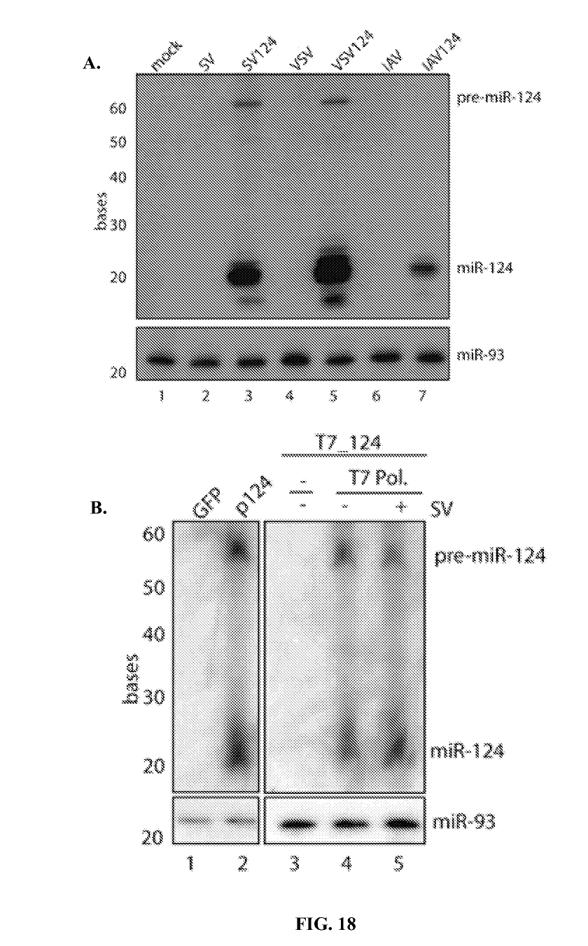

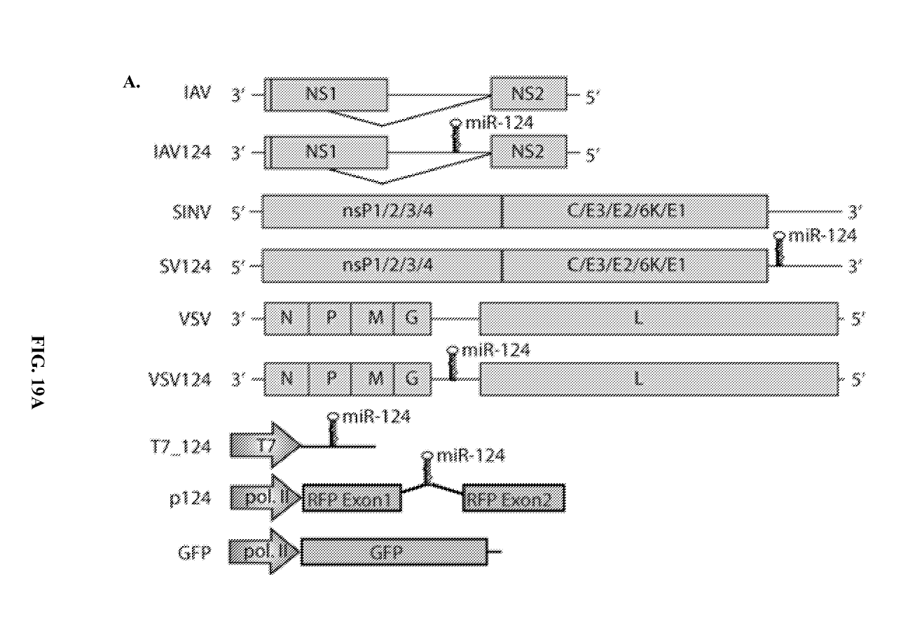

Examples 1 and 2 demonstrate the ability of influenza A virus (IAV), a nuclear negative-sense RNA virus, and Sindbis virus (SV), a cytoplasmic postive-sense RNA virus, to produce a mature functional miRNA. In both examples, insertion of the mmu-miR-124-2 locus into a non-coding region of the virus (FIG. 19A) resulted in miR-124 synthesis, corresponding with virus replication (FIG. 18A and FIG. 19B). Given that the cytoplasmic, positive-sense, RNA virus was capable of producing miRNAs, despite being inaccessible to the nuclear microprocessor, attempts were made to determine if these results could be extended to another cytoplasmic RNA virus of negative polarity. To this end, Vesicular Stomatitis Virus (VSV) was engineered to encode the mmu-mir124-2 locus as an independent virus transcript inserted between the glycoprotein (G) and large polymerase (L) genes (VSV124) (FIG. 19A). Insertion of the miRNA locus did not impede the rescue of recombinant virus and, like IAV 124 and SV124, VSV124 infection resulted in robust miR-124 synthesis (FIG. 18A). Northern blot analysis revealed detectable levels of the pre-miR-124 and mature miR-124, both products migrating to the same ˜60 nt and 20 nt products observed from plasmid-mediated nuclear expression of miR-124 (FIG. 19C). As pri-miR-124 transcripts derived from SV and VSV would both contain a 5′ cap and a 3′ poly A tail mediated by the respective virus polyermases (see Lichty et al., Trends Mol Med 10, 210-216 (2004); and Jose et al., Future Microbiol 4, 837-856 (2009)), whether these modifications were required for the generation of the mature miRNA was investigated. To this end, the mmu-miR-124-2 locus was inserted upstream of a T7 promoter and it was determined whether miR-124 expression could be observed (FIG. 18B). As T7 polymerase has neither capping nor poly A activity, any processing of miR-124 would be independent of these modifications. Surprisingly, these analyses revealed evidence for both pre-miR-124 and miR-124, derived from the cytoplasmic T7-generated primary transcript. Further, this processing, like miR-124 derived from VSV124 and SV124, was indistinguishable from plasmid-derived miR-124 (p124) (FIG. 18B). These results suggest that cytoplasmic processing of the primary miRNA is not specific to virus infection nor does it require the 5′ cap or 3′ poly A tail.

6.7.2.2 In Vivo Production of Cytoplasmic-Derived miRNAs

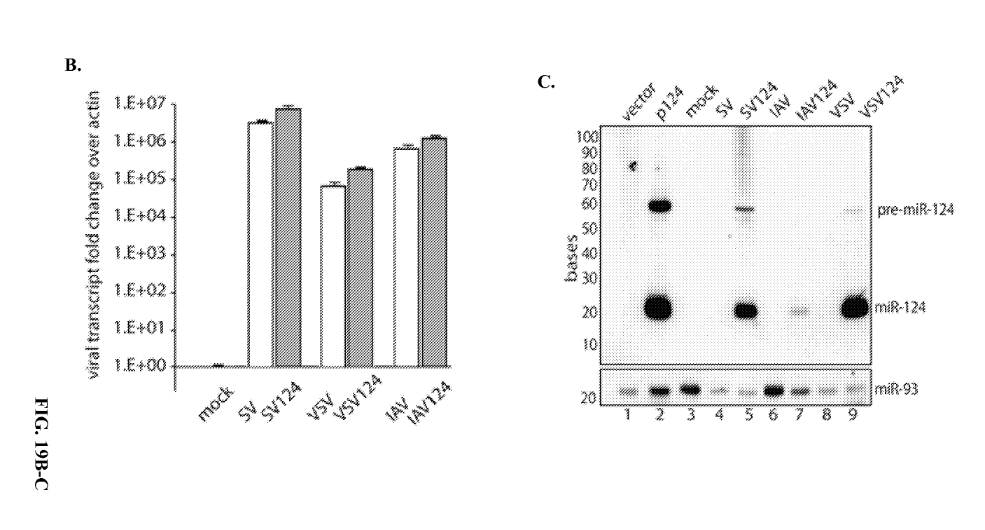

Given the evidence for in vitro pri-miRNA cytoplasmic processing from diverse RNA sources, whether this activity could be recapitulated in vivo was investigated. In an effort to compare virus-derived miRNA synthesis directly, independent of the innate antiviral response, mice deficient in Type I interferon (IFN-I) signaling were used (see Muller et al., Science 264, 1918-1921 (1994)). To this end, IFN alpha receptor I (IfnaRl) knockout mice were infected with SV124, VSV124 or IAV124, and the levels of miR-124 were measured following infection. Recombinant viruses yielded high levels of miR-124 within the lungs of the infected animals (FIG. 20). Taken together this in vivo data corroborates cytoplasmic miRNA synthesis as a bona fide biological process and not an attribute restricted to transformed cells.

6.7.2.3 Cytoplasmic-Derived miRNAs Demonstrate Star Strand Accumulation

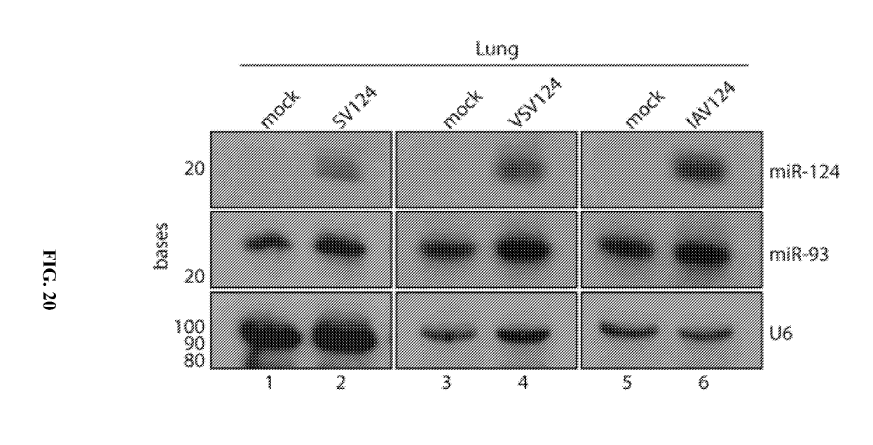

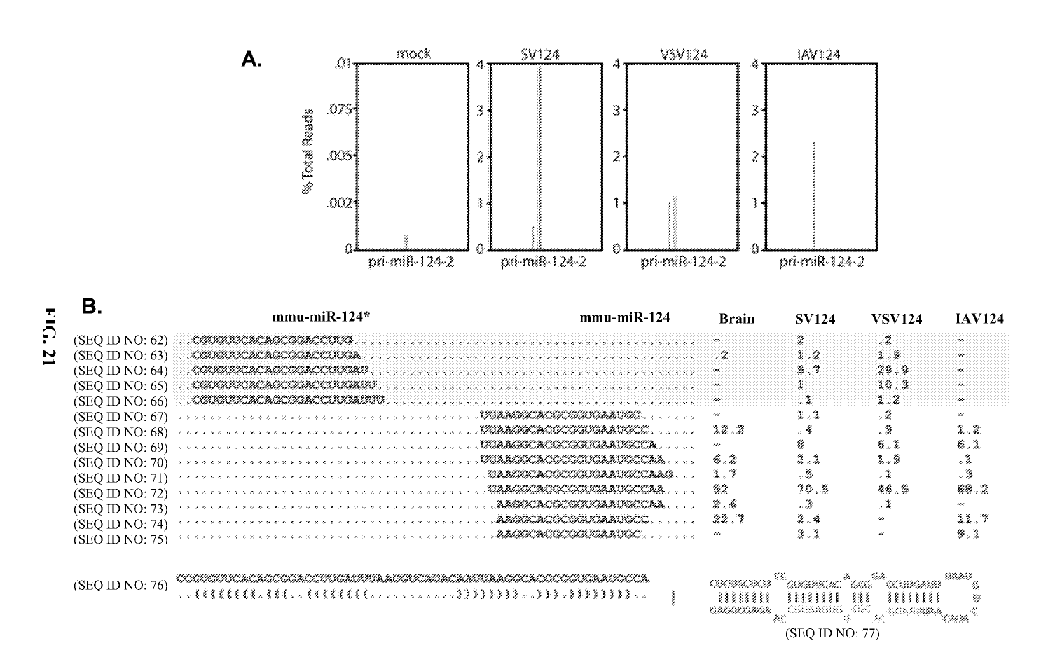

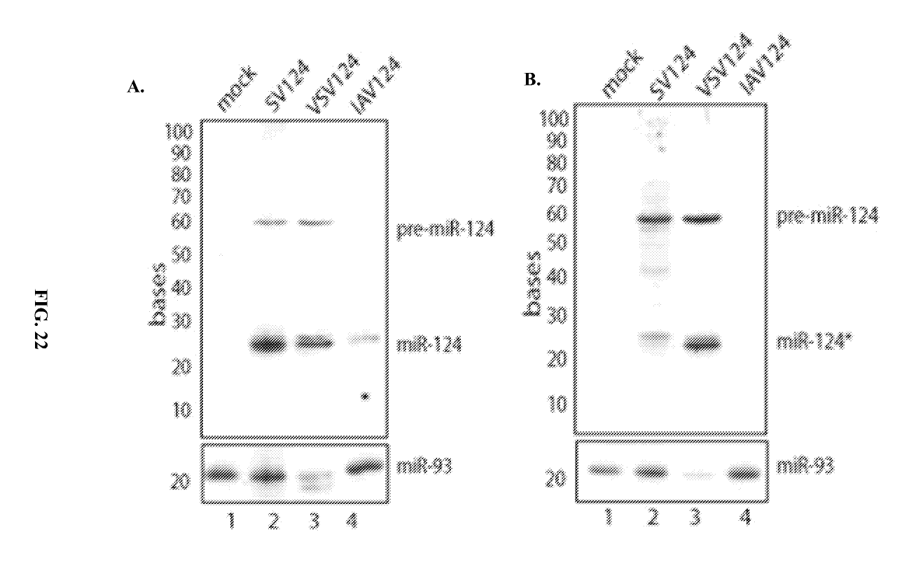

In an effort to define the sequencing characteristics of cytoplasmic-derived miR-124, wild-type (WT) murine fibroblasts were infected with each of the three virus vectors described in Section 6.7.2.2, and the small RNA fraction was analyzed by deep sequencing (FIG. 21A). Aligning captured small RNAs to the 583 nt, virus-derived, pri-miR-124 demonstrated ˜400,000 specific reads mapping to this transcript. Not surprisingly, the most abundant species mapped to the mature miR-124 product for each infection, demonstrating levels as high as 4% of the total miRNAs profiled in the cell. As miR-124 expression is limited to the brain (see Makeyev et al., Mol Cell 27, 435-448 (2007)), levels from mock-infected cells represented less than 0.001% of the total miRNAs profiled. In addition to the overall abundance of virus-derived miR-124, another striking feature of the small RNA profiling was the accumulation of star strand RNA (“miR-124*”; FIG. 21A). Restricted only to the cytoplasmic viruses, the amount of star strand captured by deep sequencing represented as much as 40% of the total RNA mapping to pre-miR-124 (FIG. 21B). This number is in stark contrast to those observed from nuclear-derived miRNA in which endogenous levels from the cerebullum (where miR-124 is abundantly expressed), represent only 0.2% of total pre-miR-124 reads. This low level of star strand RNA was also reflected by nuclear derived IAV124. Accumulation of miR-124 and miR-124* was also demonstrated by Northern blot, corroborating our deep sequencing analyses and further validating the significant production and stability of star strand from cytoplasmic pri-miR-124 processing (FIG. 22). Lastly, comparing the miR-124 sequences derived from each of these conditions demonstrated that cytoplasmic processing was produced with surprisingly high accuracy demonstrating little 5′ heterogeneity (FIG. 21B). Taken together, the accumulation of star strand provides a unique attribute to the high level of production of cytoplasmic-derived viral miR-124 synthesis, whereas the accuracy of processing strongly implicates a level of redundancy in the small RNA processing machinery.

6.7.2.4 Cytoplasmic-Derived miR-124 Associates with Ago2 to Mediate PTS

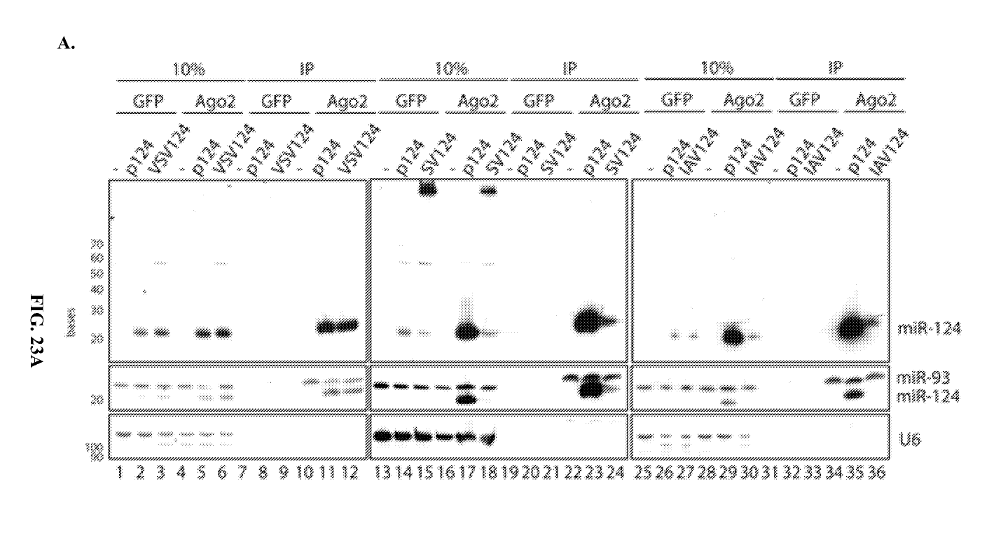

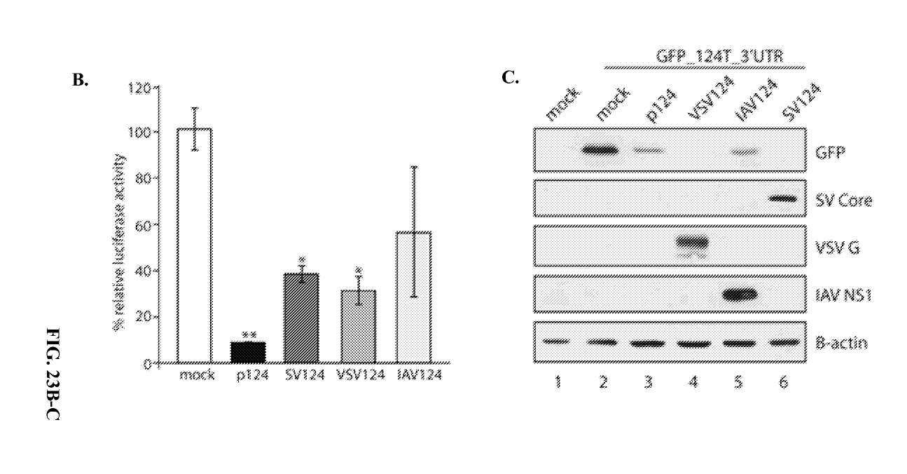

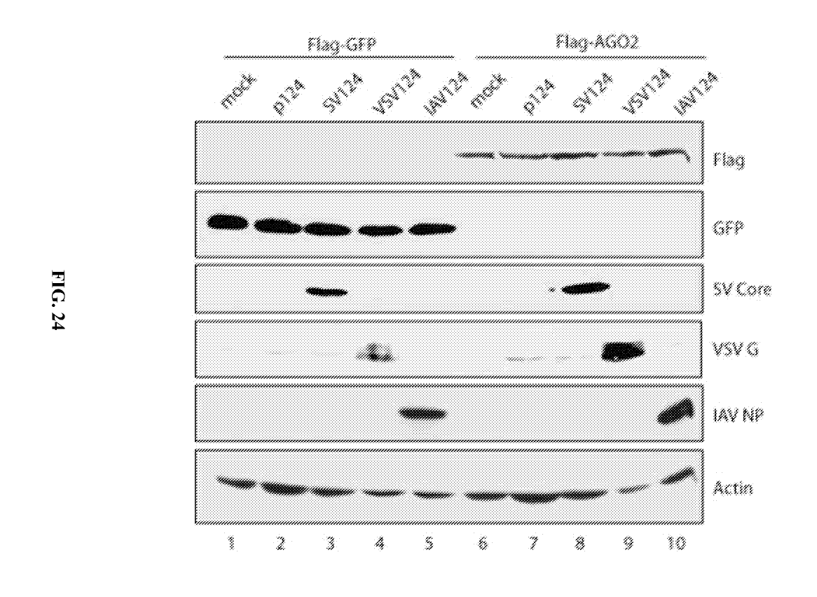

As the increased presence of miRNA star strand during cytoplasmic miRNA processing could be indicative of a lack of RISC loading and subsequent duplex separation, whether virus-derived cytoplasmic miRNAs associated with Ago2 was investigated. Cells expressing epitope tagged-Ago2 or green fluorescent protein (-GFP), mock treated or infected with miR-124-expressing viruses, were used for immunoprecipitation and associated RNA was analyzed by Northern blot (FIGS. 23A and 24). While virus-produced miR-124 was not detected from immunoprecipitation of infected, epitope-tagged, GFP-expressing cells, Ago2 associated with miR-124, regardless of intracellular origin (FIG. 23A). To examine PTS activity of the virus-produced miR-124, cells expressing firefly luciferase containing the 3′UTR of Scpl, encoding endogenous miR-124 targets (see Makeyev et al., Mol Cell 27, 435-448 (2007); and Visvanathan et al., Genes Dev 21, 744-749 (2007)), were infected with recombinant miR-124-producing viruses and compared to their parental counterparts. Infection with SV124 and VSV124 conferred significant repression of the luciferase transcript, both demonstrating ˜60% repression (FIG. 23B). Surprisingly, IAV-mediated delivery of miR-124 only trended towards a decrease in target expression but did not reach statistical significance, a result that reflects a complex dynamic between IAV infection and expression of the luciferase constructs (FIG. 23B). Given the inability of IAV 124 to significantly repress luciferase expression, a second assay to test functionality was performed. Cells expressing miR-124-targeted GFP (containing four perfect target sites in the 3′ UTR) were infected with miR-124-producing viruses and compared to mock infection or plasmid-derived miR-124. Consistent with the luciferase-based data, SV124 and VSV124 both induced dramatic silencing of GFP whereas IAV 124 demonstrated a ˜50% reduction in GFP levels, similar to plasmid-derived miRNA (FIG. 23C). Taken together, these results suggest that cytoplasmic-derived miRNAs are an effective strategy to mediate PTS.

6.7.2.5 Cytoplsmic miRNA Processing is not miRNA Specific

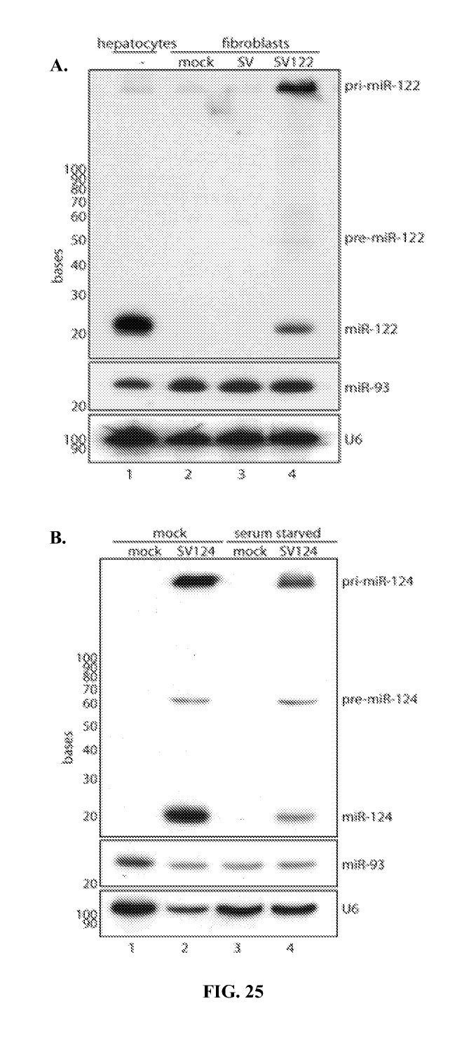

Because the analysis of cytoplsmic miRNA processing was restricted to mmu-miR-124-2, a second recombinant Sindbis virus expressing mmu-miR-122 (SV122) was engineered, as previously described (see Shapiro et al., RNA 16, 2068-2074 (2010)). Virus rescue and subsequent infection in human fibroblasts revealed robust expression of miR-122, a miRNA normally restricted to hepatocytes (see Jopling et al., Science 309, 1577-1581 (2005)), demonstrating no discernable difference to endogenous processing from hepatocytes (FIG. 25A). The synthesis of mmu-miR-124 and mmu-miR-122 strongly suggest that cytoplsmic miRNA processing is not unique to a subset of precursor miRNAs.

6.7.2.6 Cytoplasmic miRNA Processing is Cell Division Independent

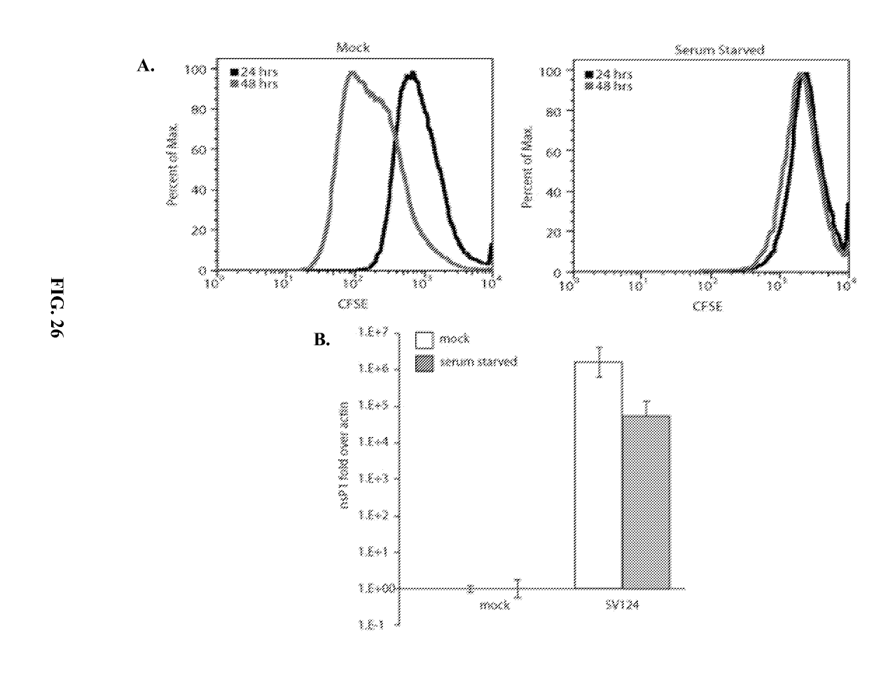

Having used Sindbis virus to demonstrate both miR-124 and miR-122 cytoplasmic synthesis and given that the complete spectrum of miRNA species (i.e. pri-, pre-, and mature-miR-124) only can be detected from SV124 infected cells, this model was chosen for further biochemical studies. Given the accuracy of SV-mediated miR-124 processing in immortalized cells, it was hypothesized that rapid division may provide cytoplasmic pri-miR-124 access to the nuclear microprocessor, leading to the generation of pre-miR-124. In an effort to address this model, cell cycle was arrested by serum starvation to ascertain whether this would abolish miR-124 production. To ensure complete arrest, cells were treated with Carboxyfluorescein succinimidyl ester (CFSE) and monitored by flow cytometry 24 and 48 hours post treatment (hpt), demonstrating a complete block in division (FIG. 26A). While SV124 infection of non-dividing cells did result in reduced levels of virus replication despite an increased inoculum size (FIG. 26B), the relative levels of miR-124 species were not affected (FIG. 25B). These data suggest that cell division is not required for cytoplasmic-mediated miRNA synthesis.

6.7.2.7 Sindbis-Produced Small RNAs are Dicer-Dependent but TRBP2-, PACT-, and Ago2-Independent

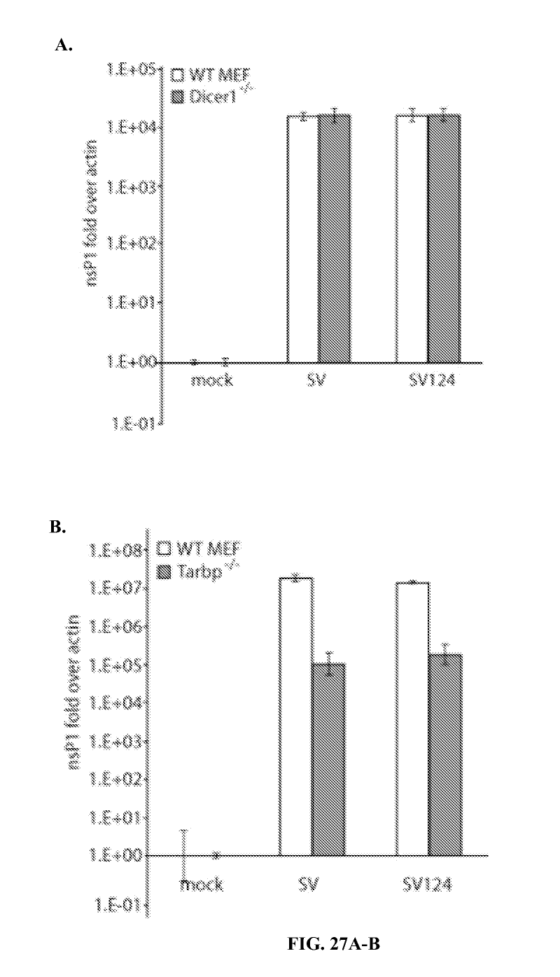

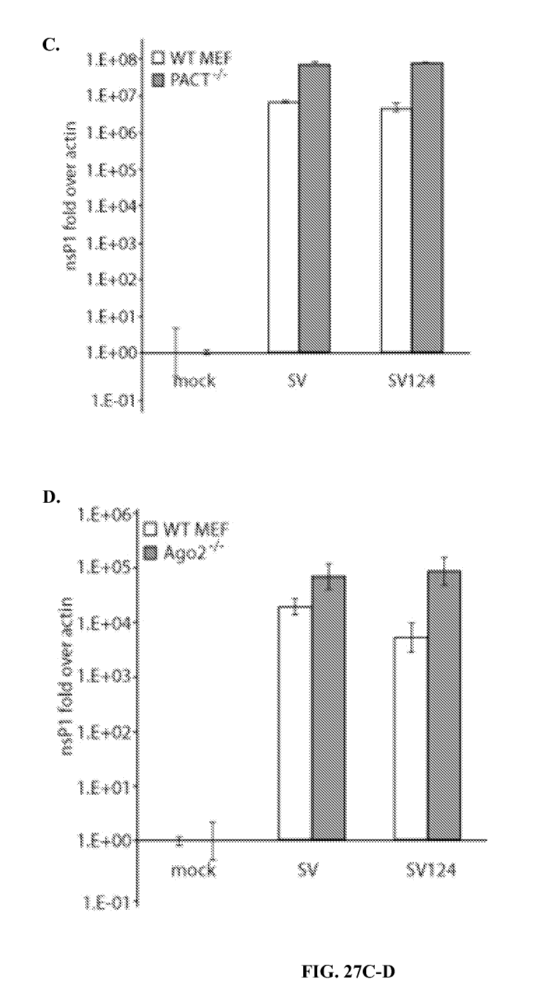

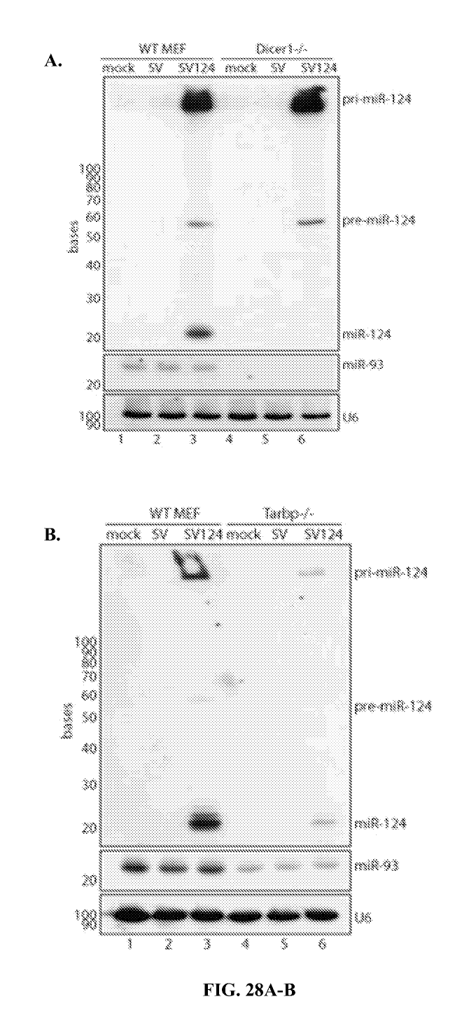

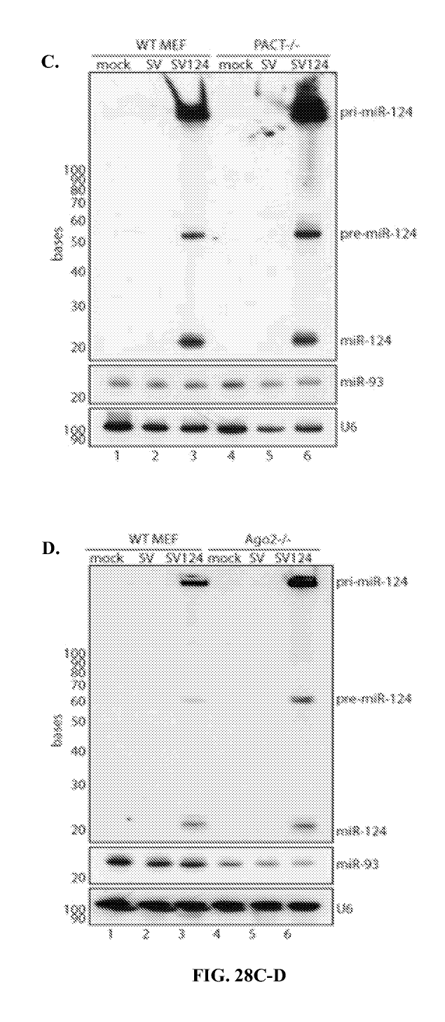

In an effort to determine whether cytoplasmic components of the canonical miRNA processing machinery are required for synthesis of Sindbis-produced small RNAs, cells disrupted in Dicer1, Tarbp, Prkra and Eif2c2 (encoding Dicer, TRBP2, PACT, and Ago2 respectively) were infected to determine if miR-124 levels were affected following infection (FIG. 27A-D). To determine whether the expression of Dicer impacted the formation of SV-generated pre-miR-124, Dicer1 deficient cells were infected at a high multiplicity of infection (MOI) to examine the visible RNA by-products that would accumulate during synthesis. Northern blot analysis from SV124 infected Dicer1 knockout cells demonstrated abundant levels of pri-miR-124 and the 60 nt pre-miRNA, with only miR-124 absent as compared to WT cells (FIG. 28A). The levels and size of pre-miR-124 was comparable to that produced in SV124-infected WT cells, suggesting Dicer is not involved in pri-miRNA cleavage of Sindbis-produced small RNAs (FIG. 28A). To evaluate TRBP2 dependency in cytoplasmic miRNA processing, Tarbp knockout cells were infected with SV124. While the levels of pri-miR-124 during SV124 infection were reduced, corresponding to a decrease in virus replication (FIG. 27B), the relative ratios of this pri-miR-124 and miR-124 were similar to infected WT cells suggesting TRBP2 independence (FIG. 28B). Similarly, SV124 infection in cells lacking another dsRNA binding protein, PACT also demonstrated no loss of miR-124 production (FIG. 28C).

Given previous results demonstrating the role of Ago2 in Dicer-independent generation of miR-451 (see Chendrimada et al., Nature 436, 740-744 (2005); Cheloufi et al., Nature 465, 584-589 (2010); and Yang et al., Proc Natl Acad Sci USA 107, 15163-15168 (2010)) and its cytoplasmic localization, whether Ago2 had a role in Sindbis-produced small RNA production despite the fact the miR-124 did not conform to the structural requirements for cleavage was investigated (see Cheloufi et al., Nature 465, 584-589 (2010); Cifuentes et al., Science 328, 1694-1698 (2010); and Yang et al., Proc Natl Acad Sci USA 107, 15163-15168 (2010)). To this end, cells deficient in Ago2 were infected to compare virus-derived cytoplasmic miR-124 synthesis to that of wild-type cells. These data demonstrated no alteration in processing despite equal levels of virus replication (FIG. 28D and FIG. 27D). Taken together, these results suggest Dicer to be a critical component for cytoplasmic miRNA processing with a possible redundant role amongst the dsRNA binding proteins PACT and TARBP.

6.7.2.8 Evidence for a Cytoplasmic, Microprocessor-Like, Function for Drosha.

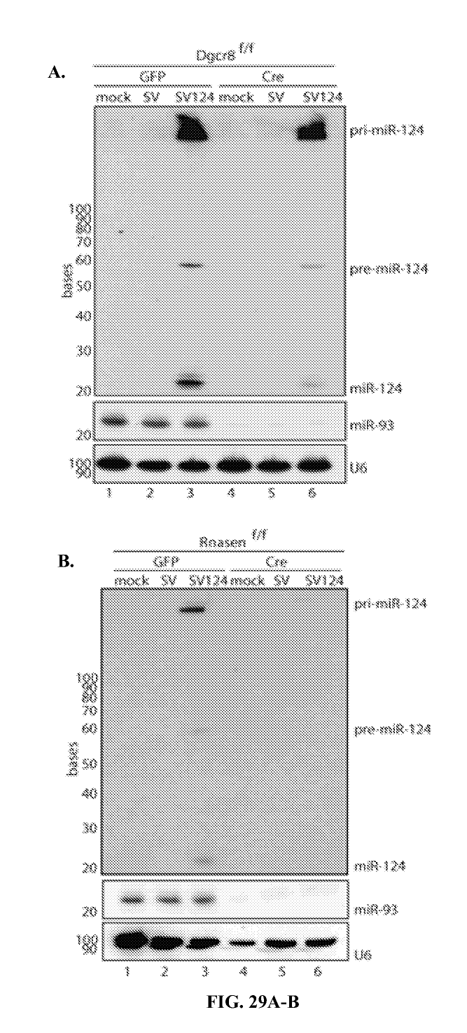

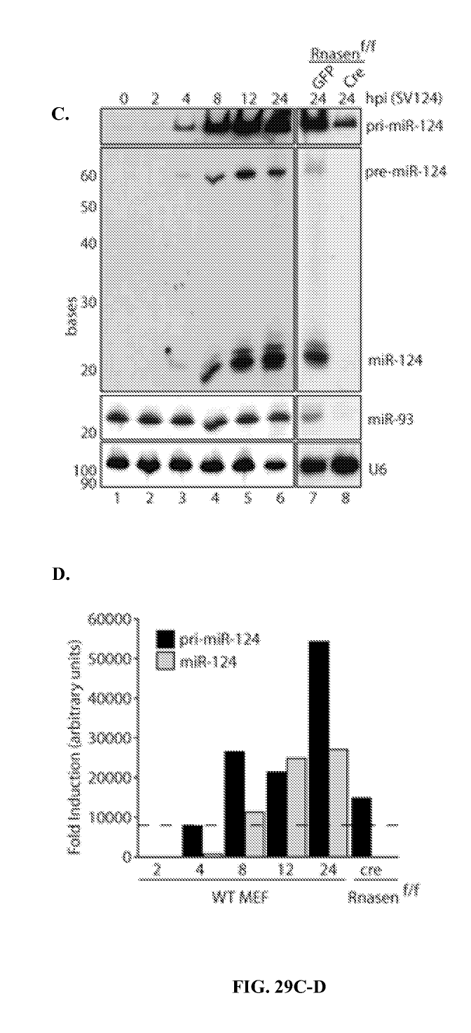

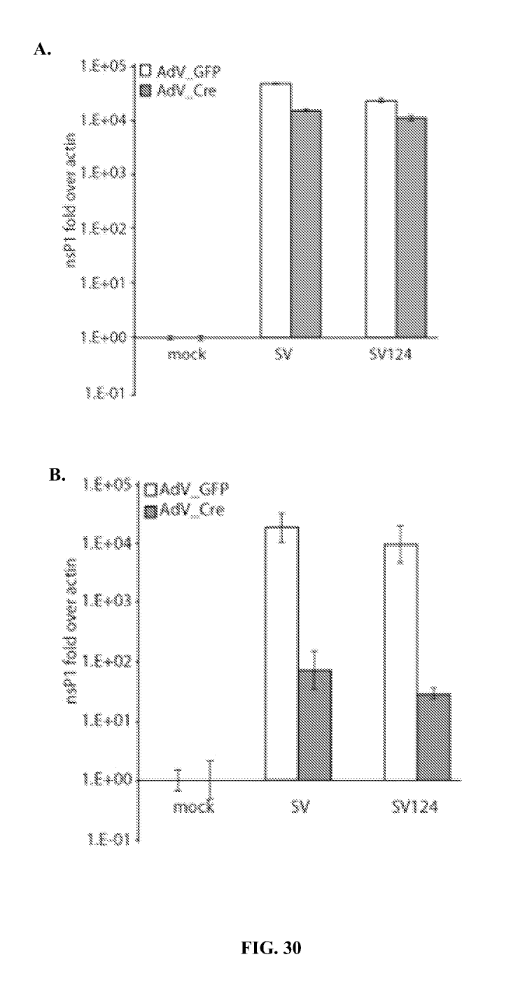

Given the cytoplasmic localization of SV124, in conjunction with the presence of a pre-miRNA produced in a Dicer-independent fashion (FIG. 25A), the nuclease responsible for production of the 60 nt pre-miRNA was investigated. To do so, SV124 small RNA synthesis in Dgcr8- and Drosha- (Rnasen^{-/-}) deficient cells was characterized (FIG. 29). As both of these fibroblast-derived cell lines are conditional knockouts, cells were initially treated with recombinant replication-deficient Adenovirus vectors expressing either GFP or GFP-Cre (AdV_GFP and AdV_Cre, respectively). Six days post-treatment of Rnasen^{fl/fl} and DGCR8^{fl/fl} cells, complete loss of endogenous miR-93 was confirmed in addition to loss of vector-dependent GFP expression, suggesting complete loss of Dgcr8 and Drosha function. (FIG. 29A). Furthermore, given the half-life of miRNAs, this data suggests that both Drosha- and DGCR8-function was abolished as early as two days post infection. To ensure complete abrogation at the time of SV124 inoculation, cells were infected 5 days post vector treatment. While SV124 small RNA synthesis was maintained despite loss of Dgcr8, loss of Drosha resulted in the inhibition of vitron synthesis (FIG. 29A-B). However, qPCR analysis and pri-miR-124 levels indicated that, while Dgcr8 deletion did not impact SV124 transcript levels, the deletion of Drosha resulted in a significant loss of replication, with nsP1 levels demonstrating a decrease greater than two orders of magnitude (FIG. 29B and FIG. 30). Therefore, in order to determine if the levels of replication were sufficient to produce a detectable miRNA, wild-type fibroblasts were mock-treated or infected with SV124 at the same MOI and miRNA species were quantified at 0, 2, 4, 8, 12, and 24 hours post-infection (FIG. 29C). Production of Sindbis-derived pri-, pre- and mature miR-124 was detected as early as 4 hours post-infection and reached maximum levels at 24 hours post-infection. Quantification of pri-miR-124 and the mature product revealed that the levels of detectable miR-124 could be predicted based on pri-miR-124 levels (FIG. 29D). This standard curve also demonstrated that the pri-miR-124 levels detected in the absence of Drosha are more than adequate to yield detectable levels of miR-124 and therefore, the absence of either pre-miR-124 or miR-124 leads to the conclusion that Sindbis small RNA production is Drosha-dependent. These data are consistent with a report that found that siRNA-mediated knockdown of Drosha, while incomplete, impacted the functionality of miRNAs produced by a recombinant Tick-borne encephalitis virus (see Rouha et al., Nucleic Acids Res 38, 8328-8337 (2010)). Taken together, these data suggest that Drosha can function within the cytoplasm, albeit with a unique dsRNA binding protein, to cleave cytoplasmic pri-miRNAs.

6.7.3 Conclusion

This example validates a novel cytoplasmic processing mechanism for the generation of mature miRNAs and demonstrates a vector-based delivery strategy for small RNA-mediated therapeutics.

### 6.8 Example 8

This example demonstrates that microRNAs can be delivered to specific tissues of interest using viral vectors.

6.8.1 Influenza A Virus

Influenza A virus (IAV) was engineered to express miR-124 (IAV124) as described in Example 1. Balb/C mice were either mock-treated (with an IAV control) or infected intranasally with 1×10⁴ plaque forming units of IAV124. At days 1, 3, and 5 post-infection, whole lung was harvested from the mice and total RNA from the harvested lungs was analyzed by Northern blot, with probing for miR-124 expression and for miR-93 expression as a control.

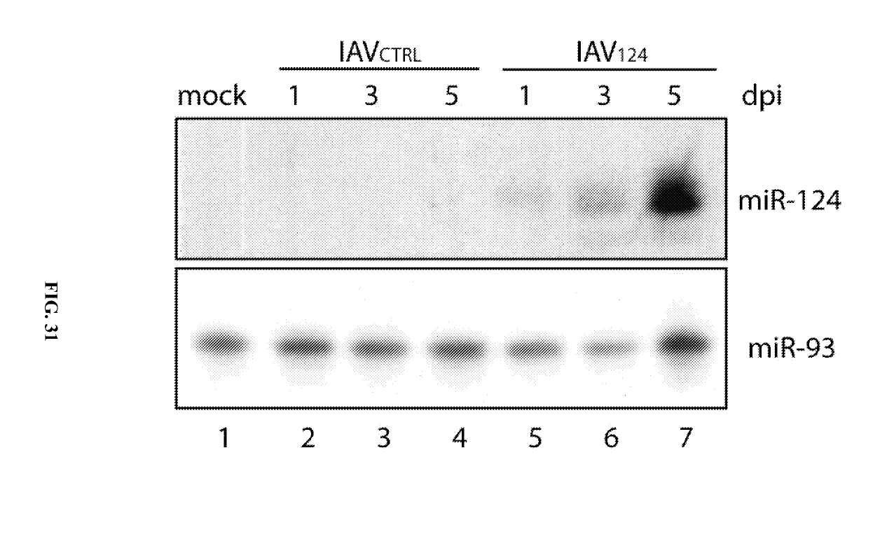

As shown in FIG. 31, IAV delivered miR-124 to the lungs of the mice, and the microRNA continued to accumulate in the lungs over the time course of the infection, consistent with the tropism of IAV.

6.8.2 Vesicular Stomatitis Virus

Vesicular stomatitis virus (VSV) was engineered to express miR-124 (VSV124) as described in Example 7. Balb/C mice were either mock-treated (with a VSV control) or infected intranasally with 1×10⁴ plaque forming units of VSV124. At 2 days post-infection, hearts, spleens, and livers were harvested from the mice and total RNA from the harvested organs was analyzed by Northern blot, with probing for miR-124 expression and for miR-93 expression as a control.

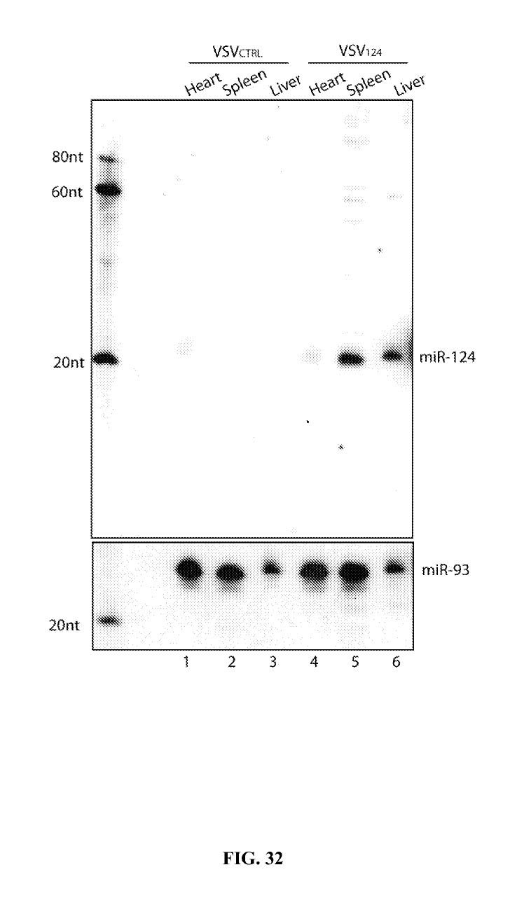

As shown in FIG. 32, the miRNA levels detected reflect a correlation between small RNA synthesis and VSV replication.

6.8.3 Conclusion

This example demonstrates that microRNAs can be delivered in vivo to multiple tissues using viral vectors.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims

TABLE 1
Sequences

| SEQ ID | | |
|---|---|---|
| NO | Description | Sequence |
| 1 | precursor of human | gcgactgtaa acatcctcga ctggaagctg tgaagccaca gatgggcttt |
| | microRNA-30a | cagtcggatg tttgcagctg c |
| 2 | precursor of human | gtggggtgtc tgtgctatgg cagccctagc acacagatac gcccagagaa |
| | microRNA-585 | agcctgaacg ttgggcgtat ctgtatgcta gggctgctgt aaca |
| 3 | microRNA-30a | UGUAAACAUCCUCGACUGGAAG |
| 4 | precursor of human | gacagtgcag tcacccataa agtagaaagc actactaaca gcactggagg |
| | micro-RNA mir-142 | gtgtagtgtt tcctacttta tggatgagtg tactgtg |
| 5 | Primer | CCATTGCCTTCTCTCCCGGGACATACTGCTGAGGA |
| | | TGTC |
| 6 | Primer | GACATCCTCAGCAGTATGTCCCGGGAGAGAAGGC |
| | | AATGG |
| 7 | Primer | GATCGCTCTTCTGGGAGCAAAAGCAGG |
| 8 | Primer | CCCCTCGAGTCAAACTTCTGACCTAATTGTTCCC |
| 9 | Primer | CGCTCGAGCACCATTGCCTTCTCTTCCAGG |
| 10 | Primer | CATCGCTCTTCTATTAGTAGAAACAAGG |
| 11 | Probe | TGGCATTCACCGCGTGCCTTAA |
| 12 | Probe | CTACCTGCACGAACAGCACTTTG |
| 13 | Probe | TCCATAAAGTAGGAAACACTACA |
| 14 | Probe | GCCATGCTAATCTTCTCTGTATC |
| 15 | Primer | AGTAGAAACAAGGGTGTTTTTTAT |
| 16 | Primer | AATGGATCCAAACACTGTGTCA |
| 17 | Primer | ATCGGAATCGCAACTAACGA |
| 18 | Primer | TTTGCGGACCAGTTCTCTCT |
| 19 | Primer | GGTTCGAGTTCTGGAAGCAG |
| 20 | Primer | GGGGATGTAGTGCTCATCGT |
| 21 | Primer | CACTGTGTCAAGCTTTCAGGACATACTG |
| 22 | Primer | CTCGTTTCTGTTTTGGAGTGAGTG |
| 23 | Primer | GGGCTTTCACCGAAGAGGGAGC |
| 24 | Primer | GTGGAGGTCTCCCATTCTCA |
| 25 | Primer | GACCAAGAACTAGGCGATGC |
| 26 | Primer | CGCTCCACTATCTGCTTTCC |
| 27 | Primer | AAGGGTGAGACACAAACTGAAGGT |
| 28 | Primer | AGTAGAAACAAGGGTGTTTTTTAT |
| 29 | Primer | CCATCGATGAGCTCCAAGAGAGGGTGAA |
| 30 | Primer | CCATCGATTCTCCCCACCCTTCCTAACT |
| 31 | Primer | TGCCTTTGTGCACTGGTATG |
| 32 | Primer | CTGGAGCAGTTTGACGACAC |
| 33 | artificial effector RNA | UUUACCAGAAACUUUGCUC |
| | that can bind to bases | |
| | 696-715 of SARS | |
| | coronavirus NP | |
| 34 | artificial effector RNA | UGGUUUCUUGUGACAUUUGCUC |
| | that can bind to bases | |
| | 9035-9056 of NFKBIA | |
| 35 | artificial effector RNA | ACCAAUUCCAUCACCAUUGUUC |
| | that can bind to bases | |
| | 605-626 of influenza | |
| | virus NP | |
| 36 | artificial effector RNA | UUUCGUAGUACAUAUUUCC |
| | that can bind to bases | |
| | 571-590 of EGFR | |
| 37 | artificial effector RNA | UAUUGUUGGAUCAUAUUCG |
| | that can bind to bases | |
| | 271-280 of KRAS | |
| 38 | artificial effector RNA | UCGUUGAGCAAGUUUACGG |
| | that can bind to bases | |
| | 370-389 of ELANE | |
| 39 | artificial effector RNA | UAACUAUCUGGCUUCACCG |
| | that can bind to bases | |
| | 937-956 of Shigella | |
| | hepA gene | |
| 40 | Probe | TGGCATTCACCGCGTGCCTTAA |
| 41 | Probe | CTACCTGCACGAACAGCACTTTG |
| 42 | Probe | GCCATGCTAATCTTCTCTGTATC |
| 43 | Probe | CAAACACCATTGTCACACTCCA |
| 44 | Probe | ATCAAGGTCCGCTGTGAACACG |
| 45 | artificial effector RNA | UACUGUCAAGAUCUUUCUGU |
| | that can bind to STAT1 | |
| 46 | hsa-miR-30a: | gcg a cuguaaacaucc uc gacuggaagcu gug a a |
| | | gccacagaugggcuuucagucggauguuugcagcugc |
| 47 | NFKBIA gene RNA | gcg acugguuucuugu gacauuugcuccu gug aa |
| | target | gccacagauggggugcaaaugacaagaaaccagcugc |
| 48 | Influenza virus | gcg acaccaauuccau caccauuguuccu gug aa |
| | nucleoprotein gene | gccacagauggggaacaauggauggaauuggugcugc |
| | RNA target | |
| 49 | EGFR gene RNA target | uggggugucug ug cuauggcagcccggaaa gauguacuacgaaag ag |
| | | aaag c cugaacguuuucguaguacauauuuccgggcugcuguaacaa |
| 50 | KRAS gene RNA target | uggggugucug ug cuauggcagccccgaau uugauccaacaauag ag |
| | | aaag c cugaacguuauuguuggaucauauucggggcugcuguaacaa |
| 51 | ELANE gene RNA | uggggugucug ug cuauggcagcccccgua uacuugcucaacgag ag |
| | target | aaag c cugaacguucguugagcaaguuuacgggggcugcuguaacaa |
| 52 | Shigella flexneri hepA | uggggugucug ug cuauggcagccccggug uagccagauaguuag ag |
| | gene RNA target | aaag c cugaacguuaacuaucuggcuucaccggggcugcuguaacaa |
| 53 | SARS coronavirus | uggggugucug ug cuauggcagcccgagca uaguuucugguaaag ag |
| | nucleoprotein gene | aaag c cugaacguuuuaccagaaacuuugcucgggcugcuguaacaa |
| | RNA target | |
| 54 | Influenza virus | gcg acaccaauuccau ca ccauuguuccu gug aa |
| | nucleoprotein gene | gccacagauggggaacaauggauggaauuggugcugc |
| | RNA target | |
| 55 | Influenza virus | gcg acugguuaaggua gguaacaaggg gug aa |
| | nucleoprotein gene | gccacagaugggcuuguuaccacuaccuuaaccagcugc |
| | RNA target | |
| 56 | RNA target for | (UUUUUUU)n...AGGU(A/G)AGUaccaauuccaucaccauugu |
| | influenza virus NP | ucGGCCGGCAUG..A AGG |
| 57 | Poly tail signal of | UUUUUUUCAUA |
| | exemplary genome of | |
| | single-stranded, | |
| | negative sense RNA | |
| | virus | |
| 58 | Subgenomic RNA | AUCUCUACGGUGGUCCUAAA |
| | promoter of exemplary | |
| | genome of single- | |
| | stranded, positive sense | |
| | RNA virus | |
| 59 | STAT1 siRNA | GACAGAAAGAGCUUGACAGUA |

| | | |
|---|---|---|
| 60 | miR-124 hairpin | CUCUGCUCU CC GUGUUCAC A GCG GA |
| | | CCUUGAUU UAAU GU |
| | | CAUACAAUUAAGGCACGCGGUGAAUGCCAAGAGC |
| | | GGAG |
| 61 | miR-124(amiRNA | CUCUGCUCU CA CAGAAAGAGCUUGAUGGUGAAU |
| | STAT1) | UAAU GUC |
| | | AUACAUUUACUGUCAAGCUCUUUCUGUUAGAGC |
| | | GGAG |
| 62 | miR-124* | CGUGUUCACAGCGGACCUUG |
| 63 | miR-124* | CGUGUUCACAGCGGACCUUGA |
| 64 | miR-124* | CGUGUUCACAGCGGACCUUGAU |
| 65 | miR-124* | CGUGUUCACAGCGGACCUUGAUU |
| 66 | miR-124* | CGUGUUCACAGCGGACCUUGAUUU |
| 67 | miR-124 | UUAAGGCACGCGGUGAAUGC |
| 68 | miR-124 | UUAAGGCACGCGGUGAAUGCC |
| 69 | miR-124 | UUAAGGCACGCGGUGAAUGCCA |
| 70 | miR-124 | UUAAGGCACGCGGUGAAUGCCAA |
| 71 | miR-124 | UAAGGCACGCGGUGAAUGCCAAG |
| 72 | miR-124 | UAAGGCACGCGGUGAAUGCCAA |
| 73 | miR-124 | AAGGCACGCGGUGAAUGCCAA |
| 74 | miR-124 | AAGGCACGCGGUGAAUGCC |
| 75 | miR-124 | AAGGCACGCGGUGAAUGC |
| 76 | miR-124-2 | CCGUGUUCACAGCGGACCUUGAUUUAAUGUCAUA |
| | | CAAUUAAGGCACGCGGUGAAUGCCA |
| 77 | miR-124 hairpin | CUCUGCUCUCCGUGUUCACAGCGGACCUUGAUUU |
| | | AAUGUCAUACAAUUAAGGCACGCGGUGAAUGCC |
| | | AAGAGCCCAG |
| 78 | SV124 genome | CGUGUUCAC AGCG AG ACCUUG |
| 79 | miR-124 | UAAGGCACGCGGUGAAUGCC |
| 80 | SV124 negative strand | GGCAUUCACCGCGUGCCUUA |
| | genome | |

1. A chimeric viral genomic segment or a chimeric viral genome, wherein the chimeric viral genomic segment or chimeric viral genome is derived from an RNA virus and wherein the chimeric viral genomic segment or chimeric viral genome comprises a heterologous RNA, wherein the heterologous RNA is transcribed in a cell to give rise to an effector RNA that interferes with the expression of a target gene in the cell, and wherein the effector RNA is miRNA, a mirtron, an shRNA, an siRNA, a piRNA, or an svRNA.

2. The chimeric virus gene segment or chimeric viral genome of claim 1, wherein the chimeric virus gene segment comprises: (a) packaging signals found in the 3′ non-coding region of an orthomyxovirus gene segment; (b) a first nucleotide sequence that forms part of an open reading frame of an orthomyxovirus virus gene; (c) a splice donor site; (d) a heterologous RNA sequence; (e) a splice acceptor site; and (f) a second nucleotide sequence that forms part of the open reading frame of the orthomyxovirus virus gene; and (g) packaging signals found in the 5′ non-coding region of an orthomyxovirus virus gene segment.

3. The chimeric virus gene segment or chimeric viral genome of claim 1, wherein the chimeric virus gene segment comprises: (a) packaging signals found in the 3′ non-coding region of an orthomyxovirus virus gene segment; (b) a first nucleotide sequence that forms part of an open reading frame of a first orthomyxovirus virus gene and a second influenza virus gene; (c) a splice donor site; (d) a second nucleotide sequence that forms part of the open reading frame of the first orthomyxovirus virus gene; (e) a heterologous RNA sequence; (e) a splice acceptor site; (f) a third nucleotide sequence that forms part of the open reading frame of the second orthomyxovirus virus gene; and (g) packaging signals found in the 5′ non-coding region of an orthomyxovirus virus gene segment.

4. The chimeric virus gene segment or chimeric viral genome of claim 3, wherein: (a) the first orthomyxovirus virus gene is the influenza virus NS1 gene and the second orthomyxovirus virus gene is the influenza virus NS2 gene; or (b) the first orthomyxovirus virus gene is the influenza virus M1 gene and the second orthomyxovirus virus gene is the influenza virus M2 gene.

5. (canceled)

6. The chimeric viral genomic segment or chimeric viral genome of claim 1, wherein the RNA virus is a segmented, single-stranded, negative sense RNA virus or a segmented double stranded RNA virus.

7. (canceled)

8. The chimeric viral genomic segment or chimeric viral genome of claim 1, wherein the RNA virus is a non-segmented, single stranded, negative sense RNA virus or a non-segmented, single stranded, positive sense RNA virus.

9. (canceled)

10. A recombinant RNA virus comprising the chimeric viral genomic segment or the chimeric viral genomic segment of claim 1.

11. A nucleic acid encoding the chimeric viral genomic segment or the chimeric viral genomic segment of claim 1.

12. The nucleic acid of claim 11, wherein the nucleic acid is DNA.

13. A method of making a recombinant RNA virus, wherein the method comprises introducing the nucleic acid of claim 12 into a cell that expresses all other components for generation of the recombinant RNA virus; and purifying the recombinant RNA virus from the supernatant of the cell.

14. (canceled)

15. A substrate comprising the chimeric viral genomic segment or chimeric viral genome of claim 1.

16. (canceled)

17. A pharmaceutical composition or immunogenic composition comprising the recombinant RNA virus of claim 10.

18. (canceled)

19. A method of treating and/or preventing a disease in a subject, the method comprising administering the recombinant RNA virus of claim 10 to the subject, wherein the effector RNA interferes with expression of a gene that is overexpressed or ectopically expressed in the disease.

20. The chimeric viral genomic segment or chimeric viral genome of claim 1, wherein the RNA virus is an orthomyxovirus, a bunyavirus, or an arenavirus.

21. The chimeric viral genomic segment or chimeric viral genome of claim 20, wherein the orthomyxovirus is influenza A virus, influenza B virus, influenza C virus, thogoto virus, or infectious salmon anemia virus; wherein the bunyavirus is bunyamwera virus, Hantaan virus, Dugbe virus, Rift Valley fever virus, or tomato spotted wilt virus; or wherein the arenavirus is Lassa virus, Junin virus, Machupo virus, or lymphocytic choriomeningitis virus.

22. The chimeric viral genomic segment or chimeric viral genome of claim 1, wherein the RNA virus is a rhabdovirus, a paramyxovirus, a filovirus, a hepatitis delta virus, a bornavirus, a picornavirus, a togavirus, a flavivirus, a coronavirus, a reovirus, a rotavirus, an orbivirus, or a Colorado tick fever virus.

23. The chimeric viral genomic segment or chimeric viral genome of claim 22, wherein the rhabdovirus is vesicular stomatitis virus (VSV), rabies, or a rabies-related virus; wherein the paramyxovirus is Newcastle Disease Virus (NDV), measles virus, mumps virus, Sendai virus, respiratory syncytial virus (RSV) or metapneumovirus; wherein the filovirus is Ebola virus or Marburg virus; or wherein the togavirus is Sindbis virus.

24. A kit comprising, in one or more containers, a chimeric viral genomic segment or chimeric viral genome, wherein the chimeric viral genomic segment or chimeric viral genome is derived from an RNA virus and wherein the chimeric viral genomic segment or chimeric viral genome comprises a heterologous RNA, wherein the heterolgous RNA is transcribed in a cell to give rise to an effector RNA that interferes with the expression of a target gene in the cell, and wherein the effector RNA is miRNA, a mirtron, an shRNA, an siRNA, a piRNA, or an svRNA.

25. (canceled)

26. A kit comprising, in one or more containers, a recombinant RNA virus, wherein said recombinant RNA virus comprises a chimeric viral genomic segment or chimeric viral genome, wherein the chimeric viral genomic segment or chimeric viral genome is derived from an RNA virus and wherein the chimeric viral genomic segment or chimeric viral genome comprises a heterologous RNA, wherein the heterolgous RNA is transcribed in a cell to give rise to an effector RNA that interferes with the expression of a target gene in the cell, and wherein the effector RNA is miRNA, a mirtron, an shRNA, an siRNA, a piRNA, or an svRNA.

27. (canceled)

28. A substrate comprising the recombinant RNA virus of claim 10.


SEQUENCE LISTING (80 sequences)

SEQ ID NO: 1 - DNA, 71 bp, Artificial Sequence
  precursor of human microRNA-30a
gcgactgtaaacatcctcgactggaagctgtgaagccacagatgggctttcagtcggatgtttgcagctgc

SEQ ID NO: 2 - DNA, 94 bp, Artificial Sequence
  precursor of human microRNA-585
gtggggtgtctgtgctatggcagccctagcacacagatacgcccagagaaagcctgaacgttgggcgtatctgtatgctagggctgctgtaaca

SEQ ID NO: 3 - RNA, 22 nt, Artificial Sequence
  microRNA-30a
uguaaacauccucgacuggaag

SEQ ID NO: 4 - DNA, 87 bp, Artificial Sequence
  precursor of human micro-RNA mir-142
gacagtgcagtcacccataaagtagaaagcactactaacagcactggagggtgtagtgtttcctactttatggatgagtgtactgtg

SEQ ID NO: 5 - DNA, 39 bp, Artificial Sequence
  Primer
ccattgccttctctcccgggacatactgctgaggatgtc

SEQ ID NO: 6 - DNA, 39 bp, Artificial Sequence
  Primer
gacatcctcagcagtatgtcccgggagagaaggcaatgg

SEQ ID NO: 7 - DNA, 27 bp, Artificial Sequence
  Primer
gatcgctcttctgggagcaaaagcagg

SEQ ID NO: 8 - DNA, 34 bp, Artificial Sequence
  Primer
cccctcgagtcaaacttctgacctaattgttccc

SEQ ID NO: 9 - DNA, 30 bp, Artificial Sequence
  Primer
cgctcgagcaccattgccttctcttccagg

SEQ ID NO: 10 - DNA, 28 bp, Artificial Sequence
  Primer
catcgctcttctattagtagaaacaagg

SEQ ID NO: 11 - DNA, 22 bp, Artificial Sequence
  Probe
tggcattcaccgcgtgccttaa

SEQ ID NO: 12 - DNA, 23 bp, Artificial Sequence
  Probe
ctacctgcacgaacagcactttg

SEQ ID NO: 13 - DNA, 23 bp, Artificial Sequence
  Probe
tccataaagtaggaaacactaca

SEQ ID NO: 14 - DNA, 23 bp, Artificial Sequence
  Probe
gccatgctaatcttctctgtatc

SEQ ID NO: 15 - DNA, 24 bp, Artificial Sequence
  Primer
agtagaaacaagggtgttttttat

SEQ ID NO: 16 - DNA, 22 bp, Artificial Sequence
  Primer
aatggatccaaacactgtgtca

SEQ ID NO: 17 - DNA, 20 bp, Artificial Sequence
  Primer
atcggaatcgcaactaacga

SEQ ID NO: 18 - DNA, 20 bp, Artificial Sequence
  Primer
tttgcggaccagttctctct

SEQ ID NO: 19 - DNA, 20 bp, Artificial Sequence
  Primer
ggttcgagttctggaagcag

SEQ ID NO: 20 - DNA, 20 bp, Artificial Sequence
  Primer
ggggatgtagtgctcatcgt

SEQ ID NO: 21 - DNA, 28 bp, Artificial Sequence
  Primer
cactgtgtcaagctttcaggacatactg

SEQ ID NO: 22 - DNA, 24 bp, Artificial Sequence
  Primer
ctcgtttctgttttggagtgagtg

SEQ ID NO: 23 - DNA, 22 bp, Artificial Sequence
  Primer
gggctttcaccgaagagggagc

SEQ ID NO: 24 - DNA, 20 bp, Artificial Sequence
  Primer
gtggaggtctcccattctca

SEQ ID NO: 25 - DNA, 20 bp, Artificial Sequence
  Primer
gaccaagaactaggcgatgc

SEQ ID NO: 26 - DNA, 20 bp, Artificial Sequence
  Primer
cgctccactatctgctttcc

SEQ ID NO: 27 - DNA, 24 bp, Artificial Sequence
  Primer
aagggtgagacacaaactgaaggt

SEQ ID NO: 28 - DNA, 24 bp, Artificial Sequence
  Primer
agtagaaacaagggtgttttttat

SEQ ID NO: 29 - DNA, 28 bp, Artificial Sequence
  Primer
ccatcgatgagctccaagagagggtgaa

SEQ ID NO: 30 - DNA, 28 bp, Artificial Sequence
  Primer
ccatcgattctccccacccttcctaact

SEQ ID NO: 31 - DNA, 20 bp, Artificial Sequence
  Primer
tgcctttgtgcactggtatg

SEQ ID NO: 32 - DNA, 20 bp, Artificial Sequence
  Primer
ctggagcagtttgacgacac

SEQ ID NO: 33 - RNA, 19 nt, Artificial Sequence
  artificial effector RNA that can bind to bases 696-715 of SARS coronavirus NP
uuuaccagaaacuuugcuc

SEQ ID NO: 34 - RNA, 22 nt, Artificial Sequence
  artificial effector RNA that can bind to bases 9035-9056 of NFKBIA
ugguuucuugugacauuugcuc

SEQ ID NO: 35 - RNA, 22 nt, Artificial Sequence
  artificial effector RNA that can bind to bases 605-626 of influenza virus NP
accaauuccaucaccauuguuc

SEQ ID NO: 36 - RNA, 19 nt, Artificial Sequence
  artificial effector RNA that can bind to bases 571-590 of EGFR
uuucguaguacauauuucc

SEQ ID NO: 37 - RNA, 19 nt, Artificial Sequence
  artificial effector RNA that can bind to bases 271-280 of KRAS
uauuguuggaucauauucg

SEQ ID NO: 38 - RNA, 19 nt, Artificial Sequence
  artificial effector RNA that can bind to bases 370-389 of ELANE
ucguugagcaaguuuacgg

SEQ ID NO: 39 - RNA, 19 nt, Artificial Sequence
  artificial effector RNA that can bind to bases 937-956 of Shigella hepA gene
uaacuaucuggcuucaccg

SEQ ID NO: 40 - DNA, 22 bp, Artificial Sequence
  Probe
tggcattcaccgcgtgccttaa

SEQ ID NO: 41 - DNA, 23 bp, Artificial Sequence
  Probe
ctacctgcacgaacagcactttg

SEQ ID NO: 42 - DNA, 23 bp, Artificial Sequence
  Probe
gccatgctaatcttctctgtatc

SEQ ID NO: 43 - DNA, 22 bp, Artificial Sequence
  Probe
caaacaccattgtcacactcca

SEQ ID NO: 44 - DNA, 22 bp, Artificial Sequence
  Probe
atcaaggtccgctgtgaacacg

SEQ ID NO: 45 - RNA, 20 nt, Artificial Sequence
  artificial effector RNA that can bind to STAT1
uacugucaagaucuuucugu

SEQ ID NO: 46 - RNA, 71 nt, Artificial Sequence
  hsa-miR-30a
gcgacuguaaacauccucgacuggaagcugugaagccacagaugggcuuucagucggauguuugcagcugc

SEQ ID NO: 47 - RNA, 71 nt, Artificial Sequence
  NFKBIA gene RNA target
gcgacugguuucuugugacauuugcuccugugaagccacagauggggugcaaaugacaagaaaccagcugc

SEQ ID NO: 48 - RNA, 71 nt, Artificial Sequence
  Influenza virus nucleoprotein gene RNA target
gcgacaccaauuccaucaccauuguuccugugaagccacagauggggaacaauggauggaauuggugcugc

SEQ ID NO: 49 - RNA, 94 nt, Artificial Sequence
  EGFR gene RNA target
uggggugucugugcuauggcagcccggaaagauguacuacgaaagagaaagccugaacguuuucguaguacauauuuccgggcugcuguaacaa

SEQ ID NO: 50 - RNA, 94 nt, Artificial Sequence
  KRAS gene RNA target
uggggugucugugcuauggcagccccgaauuugauccaacaauagagaaagccugaacguuauuguuggaucauauucggggcugcuguaacaa

SEQ ID NO: 51 - RNA, 94 nt, Artificial Sequence
  ELANE gene RNA target
uggggugucugugcuauggcagcccccguauacuugcucaacgagagaaagccugaacguucguugagcaaguuuacgggggcugcuguaacaa

SEQ ID NO: 52 - RNA, 94 nt, Artificial Sequence
  Shigella flexneri hepA gene RNA target
uggggugucugugcuauggcagccccgguguagccagauaguuagagaaagccugaacguuaacuaucuggcuucaccggggcugcuguaacaa

SEQ ID NO: 53 - RNA, 94 nt, Artificial Sequence
  SARS coronavirus nucleoprotein gene RNA target
uggggugucugugcuauggcagcccgagcauaguuucugguaaagagaaagccugaacguuuuaccagaaacuuugcucgggcugcuguaacaa

SEQ ID NO: 54 - RNA, 71 nt, Artificial Sequence; identical to SEQ ID NO: 48
  Influenza virus nucleoprotein gene RNA target

SEQ ID NO: 55 - RNA, 71 nt, Artificial Sequence
  Influenza virus nucleoprotein gene RNA target
gcgacugguuaagguagguaacaaggggugaagccacagaugggcuuguuaccacuaccuuaaccagcugc

SEQ ID NO: 56 - RNA, 50 nt, Artificial Sequence
  RNA target for influenza virus NP
uuuuuuuagguraguaccaauuccaucaccauuguucggccggcaugagg

SEQ ID NO: 57 - RNA, 11 nt, Artificial Sequence
  Poly tail signal of exemplary genome of single-stranded, negative sense RNA virus
uuuuuuucaua

SEQ ID NO: 58 - RNA, 20 nt, Artificial Sequence
  Subgenomic RNA promoter of exemplary genome of single-stranded, positive sense RNA virus
aucucuacggugguccuaaa

SEQ ID NO: 59 - RNA, 21 nt, Artificial Sequence
  STAT1 siRNA
gacagaaagagcuugacagua

SEQ ID NO: 60 - RNA, 77 nt, Artificial Sequence
  miR-124 hairpin
cucugcucuccguguucacagcggaccuugauuuaaugucauacaauuaaggcacgcggugaaugccaagagcggag

SEQ ID NO: 61 - RNA, 77 nt, Artificial Sequence
  miR-124(amiRNA STAT1)
cucugcucucacagaaagagcuugauggugaauuaaugucauacauuuacugucaagcucuuucuguuagagcggag

SEQ ID NO: 62 - RNA, 20 nt, Artificial Sequence
  mmu miR-124 star strand
cguguucacagcggaccuug

SEQ ID NO: 63 - RNA, 21 nt, Artificial Sequence
  mmu miR-124 star strand
cguguucacagcggaccuuga

SEQ ID NO: 64 - RNA, 22 nt, Artificial Sequence
  mmu miR-124 star strand
cguguucacagcggaccuugau

SEQ ID NO: 65 - RNA, 23 nt, Artificial Sequence
  mmu miR-124 star strand
cguguucacagcggaccuugauu

SEQ ID NO: 66 - RNA, 24 nt, Artificial Sequence
  mmu miR-124 star strand
cguguucacagcggaccuugauuu

SEQ ID NO: 67 - RNA, 20 nt, Artificial Sequence
  mmu miR-124
uuaaggcacgcggugaaugc

SEQ ID NO: 68 - RNA, 21 nt, Artificial Sequence
  mmu miR-124
uuaaggcacgcggugaaugcc

SEQ ID NO: 69 - RNA, 22 nt, Artificial Sequence
  mmu miR-124
uuaaggcacgcggugaaugcca

SEQ ID NO: 70 - RNA, 23 nt, Artificial Sequence
  mmu miR-124
uuaaggcacgcggugaaugccaa

SEQ ID NO: 71 - RNA, 23 nt, Artificial Sequence
  mmu miR-124
uaaggcacgcggugaaugccaag

SEQ ID NO: 72 - RNA, 22 nt, Artificial Sequence
  mmu miR-124
uaaggcacgcggugaaugccaa

SEQ ID NO: 73 - RNA, 21 nt, Artificial Sequence
  mmu miR-124
aaggcacgcggugaaugccaa

SEQ ID NO: 74 - RNA, 19 nt, Artificial Sequence
  mmu miR-124
aaggcacgcggugaaugcc

SEQ ID NO: 75 - RNA, 18 nt, Artificial Sequence
  mmu miR-124
aaggcacgcggugaaugc

SEQ ID NO: 76 - RNA, 59 nt, Artificial Sequence
  mmu miR-124
ccguguucacagcggaccuugauuuaaugucauacaauuaaggcacgcggugaaugcca

SEQ ID NO: 77 - RNA, 77 nt, Artificial Sequence
  mmu miR-124 hairpin
cucugcucuccguguucacagcggaccuugauuuaaugucauacaauuaaggcacgcggugaaugccaagagcccag

SEQ ID NO: 78 - RNA, 21 nt, Artificial Sequence
  SV124 genome
cguguucacagcgagaccuug

SEQ ID NO: 79 - RNA, 20 nt, Artificial Sequence
  miR-124
uaaggcacgcggugaaugcc

SEQ ID NO: 80 - RNA, 20 nt, Artificial Sequence
  SV124 negative strand genome
ggcauucaccgcgugccuua